SEQUENCE LISTING (86 sequences)

SEQ ID NO: 1 - DNA, 22 bp, Artificial Sequence
  Artificially synthesized primer sequence for RT-PCR
tgatcagtgtatgcgaaaaggt

SEQ ID NO: 2 - DNA, 23 bp, Artificial Sequence
  Artificially synthesized primer sequence for RT-PCR
ggtcaaggtgagtttattgtcca

SEQ ID NO: 3 - DNA, 20 bp, Artificial Sequence
  Artificially synthesized primer sequence for RT-PCR
ttgccatggacaagattcac

SEQ ID NO: 4 - DNA, 20 bp, Artificial Sequence
  Artificially synthesized primer sequence for RT-PCR
ttgtctgatccagcaagcag

SEQ ID NO: 5 - DNA, 20 bp, Artificial Sequence
  Artificially synthesized primer sequence for RT-PCR
cggaaccaaacctaagaagc

SEQ ID NO: 6 - DNA, 22 bp, Artificial Sequence
  Artificially synthesized primer sequence for RT-PCR
cttcacagccttagcagcactt

SEQ ID NO: 7 - DNA, 20 bp, Artificial Sequence
  Artificially synthesized primer sequence for RT-PCR
cctctgcaaacagaatcttg

SEQ ID NO: 8 - DNA, 25 bp, Artificial Sequence
  Artificially synthesized primer sequence for RT-PCR
aagatgtagaagcttacatagggca

SEQ ID NO: 9 - DNA, 22 bp, Artificial Sequence
  Artificially synthesized primer sequence for RT-PCR
ggaaataaggcttgctgtttgt

SEQ ID NO: 10 - DNA, 22 bp, Artificial Sequence
  Artificially synthesized primer sequence for RT-PCR
aatagtgggtttccacacatgg

SEQ ID NO: 11 - DNA, 22 bp, Artificial Sequence
  Artificially synthesized primer sequence for RT-PCR
cacaacatgcaatgtgtctgtg

SEQ ID NO: 12 - DNA, 22 bp, Artificial Sequence
  Artificially synthesized primer sequence for RT-PCR
tcctctaagacttgcaagcagc

SEQ ID NO: 13 - DNA, 22 bp, Artificial Sequence
  Artificially synthesized primer sequence for RT-PCR
catgaaggaaaacgggattatg

SEQ ID NO: 14 - DNA, 21 bp, Artificial Sequence
  Artificially synthesized primer sequence for RT-PCR
gtgcagaaagagactcatccg

SEQ ID NO: 15 - DNA, 20 bp, Artificial Sequence
  Artificially synthesized primer sequence for RT-PCR
tcccacctaacctctgcatc

SEQ ID NO: 16 - DNA, 20 bp, Artificial Sequence
  Artificially synthesized primer sequence for RT-PCR
gaagcgcgaccatttcttta

SEQ ID NO: 17 - DNA, 23 bp, Artificial Sequence
  Artificially synthesized primer sequence for RT-PCR
ggctgatacttctctcatcttgc

SEQ ID NO: 18 - DNA, 23 bp, Artificial Sequence
  Artificially synthesized primer sequence for RT-PCR
gccaccacatctttattgcatac

SEQ ID NO: 19 - DNA, 23 bp, Artificial Sequence
  Artificially synthesized primer sequence for RT-PCR
tggggttctaagacaaagaactg

SEQ ID NO: 20 - DNA, 23 bp, Artificial Sequence
  Artificially synthesized primer sequence for RT-PCR
gtgagaaaaccagtgtcaaatcc

SEQ ID NO: 21 - DNA, 23 bp, Artificial Sequence
  Artificially synthesized primer sequence for RT-PCR
tgctggtgctatttactgacgta

SEQ ID NO: 22 - DNA, 23 bp, Artificial Sequence
  Artificially synthesized primer sequence for RT-PCR
aaaagaccgtttctgactctgtg

SEQ ID NO: 23 - DNA, 20 bp, Artificial Sequence
  Artificially synthesized primer sequence for RT-PCR
aagtgacctcctctccttcc

SEQ ID NO: 24 - DNA, 23 bp, Artificial Sequence
  Artificially synthesized primer sequence for RT-PCR
cacccttcctccaagtcttttat

SEQ ID NO: 25 - DNA, 23 bp, Artificial Sequence
  Artificially synthesized primer sequence for RT-PCR
gaacccagtggatgtaacagaac

SEQ ID NO: 26 - DNA, 23 bp, Artificial Sequence
  Artificially synthesized primer sequence for RT-PCR
tactgcagagacttagctggtcc

SEQ ID NO: 27 - DNA, 23 bp, Artificial Sequence
  Artificially synthesized primer sequence for RT-PCR
acttatagtcctgcgagtctggg

SEQ ID NO: 28 - DNA, 22 bp, Artificial Sequence
  Artificially synthesized primer sequence for RT-PCR
ggcaggagagaagaacatcttg

SEQ ID NO: 29 - DNA, 22 bp, Artificial Sequence
  Artificially synthesized primer sequence for RT-PCR
catctcctttgtttcgatagga

SEQ ID NO: 30 - DNA, 22 bp, Artificial Sequence
  Artificially synthesized primer sequence for RT-PCR
gatcactgtgggtcttaagcaa

SEQ ID NO: 31 - DNA, 20 bp, Artificial Sequence
  Artificially synthesized primer sequence for RT-PCR
gggctggtgcagatctactt

SEQ ID NO: 32 - DNA, 23 bp, Artificial Sequence
  Artificially synthesized primer sequence for RT-PCR
tccaacatctgttgagtgacagt

SEQ ID NO: 33 - DNA, 23 bp, Artificial Sequence
  Artificially synthesized primer sequence for RT-PCR
cactcagaattcttacctcccct

SEQ ID NO: 34 - DNA, 23 bp, Artificial Sequence
  Artificially synthesized primer sequence for RT-PCR
gtgatgtgaagcaaggtagttcc

SEQ ID NO: 35 - DNA, 20 bp, Artificial Sequence
  Artificially synthesized primer sequence for RT-PCR
gccaaaaatggctctctagg

SEQ ID NO: 36 - DNA, 23 bp, Artificial Sequence
  Artificially synthesized primer sequence for RT-PCR
cagacacgcacttgtggtttatt

SEQ ID NO: 37 - DNA, 21 bp, Artificial Sequence
  Artificially synthesized primer sequence for RT-PCR
gtgtccacttagagcctcacg

SEQ ID NO: 38 - DNA, 23 bp, Artificial Sequence
  Artificially synthesized primer sequence for RT-PCR
atccttcttcctatacttccccc

SEQ ID NO: 39 - DNA, 20 bp, Artificial Sequence
  Artificially synthesized primer sequence for RT-PCR
tttaatcaggccctgtctgc

SEQ ID NO: 40 - DNA, 23 bp, Artificial Sequence
  Artificially synthesized primer sequence for RT-PCR
ggggtatagaaatggaatggaga

SEQ ID NO: 41 - DNA, 23 bp, Artificial Sequence
  Artificially synthesized primer sequence for RT-PCR
ctggaagaagaaggaacaggtct

SEQ ID NO: 42 - DNA, 23 bp, Artificial Sequence
  Artificially synthesized primer sequence for RT-PCR
ggttgctgagattttatctgtgg

SEQ ID NO: 43 - DNA, 23 bp, Artificial Sequence
  Artificially synthesized primer sequence for RT-PCR
caaatgctctgctttgtactcct

SEQ ID NO: 44 - DNA, 23 bp, Artificial Sequence
  Artificially synthesized primer sequence for RT-PCR
catgaatgagcctgaaatagtcc

SEQ ID NO: 45 - DNA, 23 bp, Artificial Sequence
  Artificially synthesized primer sequence for RT-PCR
cgggaggattgtaagatactgtg

SEQ ID NO: 46 - DNA, 23 bp, Artificial Sequence
  Artificially synthesized primer sequence for RT-PCR
acttctcatgagttcagcctcag

SEQ ID NO: 47 - DNA, 23 bp, Artificial Sequence
  Artificially synthesized primer sequence for RT-PCR
gtagatgtggggacaacagagag

SEQ ID NO: 48 - DNA, 23 bp, Artificial Sequence
  Artificially synthesized primer sequence for RT-PCR
tttaaagtcaccttaggttgggg

SEQ ID NO: 49 - DNA, 22 bp, Artificial Sequence
  Artificially synthesized primer sequence for RT-PCR
gtttttgtggggactaagagtg

SEQ ID NO: 50 - DNA, 23 bp, Artificial Sequence
  Artificially synthesized primer sequence for RT-PCR
ggaggaagtagctagaagctaag

SEQ ID NO: 51 - DNA, 22 bp, Artificial Sequence
  Artificially synthesized primer sequence for RT-PCR
cttttcccacaagaaccatttc

SEQ ID NO: 52 - DNA, 23 bp, Artificial Sequence
  Artificially synthesized primer sequence for RT-PCR
ctggtgtaatcagacaccacgta

SEQ ID NO: 53 - DNA, 23 bp, Artificial Sequence
  Artificially synthesized primer sequence for RT-PCR
ctcatctgtaccctcactgggat

SEQ ID NO: 54 - DNA, 22 bp, Artificial Sequence
  Artificially synthesized primer sequence for RT-PCR
ctaaagtctcccagtttcccct

SEQ ID NO: 55 - DNA, 20 bp, Artificial Sequence
  Artificially synthesized primer sequence for RT-PCR
aagccagagagcctttcctc

SEQ ID NO: 56 - DNA, 23 bp, Artificial Sequence
  Artificially synthesized primer sequence for RT-PCR
cggtattcttaacacatcttgcc

SEQ ID NO: 57 - DNA, 23 bp, Artificial Sequence
  Artificially synthesized primer sequence for RT-PCR
acctaacgtttgtgccttatgtg

SEQ ID NO: 58 - DNA, 22 bp, Artificial Sequence
  Artificially synthesized primer sequence for RT-PCR
aggttggaagatccatttcctt

SEQ ID NO: 59 - DNA, 23 bp, Artificial Sequence
  Artificially synthesized primer sequence for RT-PCR
cttgggtctgtaacaaagcattc

SEQ ID NO: 60 - DNA, 23 bp, Artificial Sequence
  Artificially synthesized primer sequence for RT-PCR
aaggattatgaggaggttggtgt

SEQ ID NO: 61 - DNA, 20 bp, Artificial Sequence
  Artificially synthesized primer sequence for RT-PCR
ttagctgtgctcgcgctact

SEQ ID NO: 62 - DNA, 20 bp, Artificial Sequence
  Artificially synthesized primer sequence for RT-PCR
tcacatggttcacacggcac

SEQ ID NO: 63 - DNA, 29 bp, Artificial Sequence
  Artificially synthesized primer sequence for RT-PCR
cgcggatcccactatgacatcgccccagc

SEQ ID NO: 64 - DNA, 30 bp, Artificial Sequence
  Artificially synthesized primer sequence for RT-PCR
ccgctcgaggcaaaaaaagtcacagcacgg

SEQ ID NO: 65 - DNA, 22 bp, Artificial Sequence
  Artificially synthesized primer sequence for PCR
tggtagccaagtgcaggttata

SEQ ID NO: 66 - DNA, 22 bp, Artificial Sequence
  Artificially synthesized primer sequence for PCR
ccaaagggtttctgcagtttca

SEQ ID NO: 67 - DNA, 30 bp, Artificial Sequence
  Artificially synthesized primer sequence for PCR
tgcggatccagagcagattgtactgagagt

SEQ ID NO: 68 - DNA, 29 bp, Artificial Sequence
  Artificially synthesized primer sequence for PCR
ctctatctcgagtgaggcggaaagaacca

SEQ ID NO: 69 - DNA, 48 bp, Artificial Sequence
  Artificially synthesized primer sequence for PCR
tttaagcttgaagaccatttttggaaaaaaaaaaaaaaaaaaaaaaca

SEQ ID NO: 70 - DNA, 34 bp, Artificial Sequence
  Artificially synthesized primer sequence for PCR
tttaagcttgaagacatgggaaagagtggtctca

SEQ ID NO: 71 - DNA, 51 bp, Artificial Sequence
  Artificially synthesized oligonucleotide sequence for siRNA
caccgaggctgatggcaagaaacttcaagagagtttcttgccatcagcctc

SEQ ID NO: 72 - DNA, 51 bp, Artificial Sequence
  Artificially synthesized oligonucleotide sequence for siRNA
aaaagaggctgatggcaagaaactctcttgaagtttcttgccatcagcctc

SEQ ID NO: 73 - DNA, 51 bp, Artificial Sequence
  Artificially synthesized oligonucleotide sequence for siRNA
caccgagatgaatctcacggaatttcaagagaattccgtgagattcatctc

SEQ ID NO: 74 - DNA, 51 bp, Artificial Sequence
  Artificially synthesized oligonucleotide sequence for siRNA
aaaagagatgaatctcacggaattctcttgaaattccgtgagattcatctc

SEQ ID NO: 75 - DNA, 51 bp, Artificial Sequence
  Artificially synthesized oligonucleotide sequence for siRNA
caccgtaggacacttcttattcgttcaagagacgaataagaagtgtcctac

SEQ ID NO: 76 - DNA, 51 bp, Artificial Sequence
  Artificially synthesized oligonucleotide sequence for siRNA
aaaagtaggacacttcttattcgtctcttgaacgaataagaagtgtcctac

SEQ ID NO: 77 - DNA, 51 bp, Artificial Sequence
  Artificially synthesized oligonucleotide sequence for siRNA
caccgtgatgcattgttccttcattcaagagatgaaggaacaatgcatcac

SEQ ID NO: 78 - DNA, 51 bp, Artificial Sequence
  Artificially synthesized oligonucleotide sequence for siRNA
aaaagtgatgcattgttccttcatctcttgaatgaaggaacaatgcatcac

SEQ ID NO: 79 - DNA, 51 bp, Artificial Sequence
  Artificially synthesized oligonucleotide sequence for siRNA
caccgcttggctgtagatatctcttcaagagagagatatctacagccaagc

SEQ ID NO: 80 - DNA, 51 bp, Artificial Sequence
  Artificially synthesized oligonucleotide sequence for siRNA
aaaagcttggctgtagatatctctctcttgaagagatatctacagccaagc

SEQ ID NO: 81 - DNA, 51 bp, Artificial Sequence
  Artificially synthesized oligonucleotide sequence for siRNA
caccgaagcagcacgacttcttcttcaagagagaagaagtcgtgctgcttc

SEQ ID NO: 82 - DNA, 51 bp, Artificial Sequence
  Artificially synthesized oligonucleotide sequence for siRNA
aaaagaagcagcacgacttcttctctcttgaagaagaagtcgtgctgcttc

SEQ ID NO: 83 - DNA, 51 bp, Artificial Sequence
  Artificially synthesized oligonucleotide sequence for siRNA
caccgtgcgctgctggtgccaactctcttgaagttggcaccagcagcgcac

SEQ ID NO: 84 - DNA, 51 bp, Artificial Sequence
  Artificially synthesized oligonucleotide sequence for siRNA
aaaagtgcgctgctggtgccaacttcaagagagttggcaccagcagcgcac

SEQ ID NO: 85 - DNA, 19 bp, Artificial Sequence
  Target sequence for siRNA
gtaggacacttcttattcg

SEQ ID NO: 86 - DNA, 19 bp, Artificial Sequence
  Target sequence for siRNA
gtgatgcattgttccttca

(19) United States
(12) Patent Application Publication
(10) Pub. No.: US 2006/0194199 A1
(43) Pub. Date: Aug. 31, 2006

(54) Method for diagnosing testicular seminomas

(75) Inventors: Yusuke Nakamura, Yokohama-shi (JP); Toyomasa Katagiri, Shinagawa-ku (JP)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignees: Oncotherapy Science, Inc., Kanagawa (JP); The University of Tokyo, Tokyo (JP)

(21) Appl. No.: 10/529,593

(22) PCT Filed: Sep. 12, 2003
(86) PCT No.: PCT/JP03/11711

§ 371 Date: Apr. 5, 2006

Related U.S. Application Data
(60) Provisional application No. 60/414,677, filed on Sep. 30, 2002.

(51) Int. Cl. C12Q 1/68; A61K 48/00

(52) U.S. Cl. 435/6; 514/44

(57) ABSTRACT

Objective methods for detecting and diagnosing testicular seminoma (TS) arc described herein. In one embodiment, the diagnostic method involves the determining a expression level of TS -associated gene that discriminate between TS and nomal cell. The present invention further provides methods of screening for therapeutic agents useful in the treatment of TS, methods of treating TS and method of vaccinating a subject against TS.

### PRIORITY INFOMATION

This application claims priority to U.S. Provisional Application Ser. No.60/414,677, filed Sep. 30, 2002.

### FIELD OF THE INVENTION

The invention relates to methods of diagnosing testicular seminomas.

### BACKGROUND OF THE INVENTION

Although testicular germ cell tumors (TGCTs) account for around 1-2% of all cancers in males, they are the most common cancers found in males aged 20 to 40 year-old age group(1), and the incidence has been markedly increasing over the past several decades(2,3). TGCTs are divided into two main histological types, the seminoma, which resembles the undifferentiated germ cells and the nonseminoma, which can resemble both embryonic and extra-embryonic tissues due to their ability to differentiate down either pathway(7). Seminoma is the most common histologic testis tumor in TGCTs and account for approximately 60% to 65% of all TGCTs(8). Currently, Alpha-fetoprotein (AFP), human beta-subunit chorionic gonadotropin (HCGβ) and lactic dehydrogenase (LDH) have been used as diagnostic tumor markers of TGCTs (9). However, a specific tumor marker of seminoma without syncytiotrophoblastic giant cells has not been identified.

cDNA microarray technologies have enabled to obtain comprehensive profiles of gene expression in normal and malignant cells, and compare the gene expression in malignant and corresponding normal cells (Okabe et al., Cancer Res 61:2129-37 (2001); Kitahara et al., Cancer Res 61: 3544-9 (2001); Lin et al., Oncogene 21:4120-8 (2002); Hasegawa et al., Cancer Res 62:7012-7 (2002)). This approach enables to disclose the complex nature of cancer cells, and helps to understand the mechanism of carcinogenesis. Identification of genes that are deregulated in tumors can lead to more precise and accurate diagnosis of individual cancers, and to develop novel therapeutic targets (Bienz and Clevers, Cell 103:311-20 (2000)). To disclose mechanisms underlying tumors from a genome-wide point of view, and discover target molecules for diagnosis and development of novel therapeutic drugs, the present inventors have been analyzing the expression profiles of tumor cells using a cDNA microarray of 23040 genes (Okabe et al., Cancer Res 61:2129-37 (2001); Kitahara et al., Cancer Res 61:3544-9 (2001); Lin et al., Oncogene 21:4120-8 (2002); Hasegawa et al., Cancer Res 62:7012-7 (2002)).

Studies designed to reveal mechanisms of carcinogenesis have already facilitated identification of molecular targets for anti-tumor agents. For example, inhibitors of farnexyltransferase (FTIs) which were originally developed to inhibit the growth-signaling pathway related to Ras, whose activation depends on posttranslational farnesylation, has been effective in treating Ras-dependent tumors in animal models (He et al., Cell 99:335-45 (1999)). Clinical trials on human using a combination or anti-cancer drugs and anti-HER2 monoclonal antibody, trastuzumab, have been conducted to antagonize the proto-oncogene receptor HER2/neu; and have been achieving improved clinical response and overall survival of breast-cancer patients (Lin et al., Cancer Res 61:6345-9 (2001)). A tyrosine kinase inhibitor, STI-571, which selectively inactivates bcr-abl fusion proteins, has been developed to treat chronic myelogenous leukemias wherein constitutive activation of bcr-abl tyrosine kinase plays a crucial role in the transformation of leukocytes. Agents of these kinds are designed to suppress oncogenic activity of specific gene products (Fujita et al., Cancer Res 61:7722-6 (2001)). Therefore, gene products commonly up-regulated in cancerous cells may serve as potential targets for developing novel anti-cancer agents.

It has been demonstrated that CD8+ cytotoxic T lymphocytes (CTLs) recognize epitope peptides derived from tumor-associated antigens (TAAs) presented on MHC Class I molecule, and lyse tumor cells. Since the discovery of MAGE family as the first example of TAAs, many other TAAs have been discovered using immunological approaches (Boon, Int J Cancer 54: 177-80 (1993); Boon and van der Bruggen, J Exp Med 183: 725-9 (1996); van der Bruggen et al., Science 254: 1643-7 (1991); Brichard et al., J Exp Med 178: 489-95 (1993); Kawakami et al., J Exp Med 180: 347-52 (1994)). Some of the discovered TAAs are now in the stage of clinical development as targets of immunotherapy. TAAs discovered so far include MAGE (van der Bruggen et al., Science 254: 1643-7 (1991)), gp10 (Kawakami et al., J Exp Med 180: 347-52 (1994)), SART (Shichijo et al., J Exp Med 187: 277-88 (1998)), and NY-ESO-1 (Chen et al., Proc Natl Acad Sci USA 94: 1914-8 (1997)). On the other hand, gene products which had been demonstrated to be specifically overexpressed in tumor cells, have been shown to be recognized as targets inducing cellular immune responses. Such gene products include p53 (Umano et al., Brit J Cancer 84: 1052-7 (2001)), HER2/neu (Tanaka et al., Brit J Cancer 84: 94-9 (2001)), CEA (Nukaya et al., Int J Cancer 80: 92-7 (1999)), and so on.

In spite of significant progress in basic and clinical research concerning TAAs (Rosenbeg et al., Nature Med 4: 321-7 (1998); Mukhedji et al., Proc Natl Acad Sci USA 92: 8078-82 (1995); Hu et al., Cancer Res 56: 2479-83 (1996)), only limited number of candidate TAAs for the treatment of adenocarcinomas, including colorectal cancer, are available. TAAs abundantly expressed in cancer cells, and at the same time which expression is restricted to cancer cells would be promising candidates as immunotherapeutic targets. Further, identification of new TAAs inducing potent and specific antitumor immune responses is expected to encourage clinical use of peptide vaccination strategy in various types of cancer (Boon and can der Bruggen, J Exp Med 183: 725-9 (1996); van der Bruggen et al., Science 254: 1643-7 (1991); Brichard et al., J Exp Med 178: 489-95 (1993); Kawakami et al., J Exp Med 180: 347-52 (1994); Shichijo et al., J Exp Med 187: 277-88 (1998); Chen et al., Proc Natl Acad Sci USA 94: 1914-8 (1997); Harris, J Natl Cancer Inst 88: 1442-5 (1996); Butterfield et al., Cancer Res 59: 3134-42 (1999); Vissers et al., Cancer Res 59: 5554-9 (1999); van der Burg et al., J Immunol 156: 3308-14 (1996); Tanaka et al., Cancer Res 57: 4465-8 (1997); Fujie et al., Int J Cancer 80: 169-72 (1999); Kikuchi et al., Int J Cancer 81: 459-66 (1999); Oiso et al., Int J Cancer 81: 387-94 (1999)).

It has been repeatedly reported that peptide-stimulated peripheral blood mononuclear cells (PBMCs) from certain healthy donors produce significant levels of IFN-γ in response to the peptide, but rarely exert cytotoxicity against tumor cells in an HLA-A24 or -A0201 restricted manner in ⁵¹Cr-release assays (Kawano et al., Cancer Res 60: 3550-8 (2000); Nishizaka et al., Cancer Res 60: 4830-7 (2000); Tamura et al., Jpn J Cancer Res 92: 762-7 (2001)). However, both of HLA-A24 and HLA-A0201 are one of the popular HLA alleles in Japanese, as well as Caucasian (Date et al., Tissue Antigens 47: 93-101 (1996); Kondo et al., J Immunol 155: 4307-12 (1995); Kubo et al., J Immunol 152: 3913-24 (1994); Imanishi et al., Proceeding of the eleventh International Hictocompatibility Workshop and Conference Oxford University Press, Oxford, 1065 (1992); Williams et al., Tissue Antigen 49: 129 (1997)). Thus, antigenic peptides of carcinomas presented by these HLAs may be especially useful for the treatment of carcinomas among Japanese and Caucasian. Further, it is known that the induction of low-afinity CTL in vitro usually results from the use of peptide at a high concentration, generating a high level of specific peptide/MHC complexes on antigen presenting cells (APCs), which will effectively activate these CTL (Alexander-Miller et al., Proc Natl Acad Sci USA 93: 4102-7 (1996)).

PYRIN-containing Apaf-1-like proteins (PYPAFs) are recently identified proteins (37). It has been reported that 14 PYPAFs genes exist in Homo sapiens (38). All of PYPAF proteins which contains leucine-rich repeat, PYRIN, NACHT and NACHT-associated domains were thought to function in apoptotic and inflammatory signaling pathways. PYRIN domain at the N terminus has been reported to be associated with protein-protein interaction (38). In addition, NACHT domain has sequence homology with the nucleotide-binding motif of apoptotic protease-activating factor-1 (APAF-1), and are predicted to bind ATP(37). However, PYRIN-containing Apaf-1-like proteins have never been involved in tumorigenesis.

### SUMMARY OF THE INVENTION

The invention is based on the discovery of a pattern of gene expression correlated with testicular seminomas (TS). The genes that are differentially expressed in TS are collectively referred to herein as “TS nucleic acids” or “TS polynucleotides” and the corresponding encoded polypeptides are referred to as “TS polypeptides” or “TS proteins.”

Accordingly, the invention features a method of diagnosing or determining a predisposition to TS in a subject by determining an expression level of a TS-associated gene in a patient derived biological sample, such as tissue sample. By TS associated gene is meant a gene that is characterized by an expression level which differs in a cell obtained from a testicular germ cell tumor cell compared to a normal cell. A normal cell is one obtained from testis tissue. A TS-associated gene is one or more of TS 1-939. An alteration, e.g., increase or decrease of the level of expression of the gene compared to a normal control level of the gene indicates that the subject suffers from or is at risk of developing TS.

By normal control level is meant a level of gene expression detected in a normal, healthy individual or in a population of individuals known not to be suffering from TS. A control level is a single expression pattern derived from a single reference population or from a plurality of expression patterns. For example, the control level can be a database of expression patterns from previously tested cells. A normal individual is one with no clinical symptoms of TS and no family history of TS.

An increase in the level of TS 1-346 detected in a test sample compared to a normal control level indicates the subject (from which the sample was obtained) suffers from or is at risk of developing TS. In contrast, a decrease in the level of TS 347-939 detected in a test sample compared to a normal control level indicates said subject suffers from or is at risk of developing TS.

Alternatively, expression of a panel of TS-associated genes in the sample is compared to a TS control level of the same panel of genes. By TS control level is meant the expression profile of the TS-associated genes found in a population suffering from TS.

Gene expression is increased or decreased 10%, 25%, 50% compared to the control level. Alternately, gene expression is increased or decreased 0.1, 0.2, 1, 2, 5, 10 or more fold compared to the control level. Expression is determined by detecting hybridization, e.g., on an array, of a TS-associated gene probe to a gene transcript of the patient-derived tissue sample.

The patient derived tissue sample is any tissue from a test subject, e.g., a patient known to or suspected of having TS. For example, the tissue contains a testicular germ cell tumor cell. For example, the tissue is a cell from testis.

The invention also provides a TS reference expression profile of a gene expression level of two or more of TS 1-346. Alternatively, the invention provides a TS reference expression profile of the levels of expression of two or more of TS 1-346 or TS 347-939.

The invention further provides methods of identifing an agent that inhibits or enhances the expression or activity of a TS-associated gene, e.g TS 1-939 by contacting a test cell expressing a TS associated gene with a test agent and determining the expression level of the TS associated gene. The test cell is a testis cell such as a testis cell from a testicular germ cell tumor. A decrease of the level compared to a normal control level of the gene indicates that the test agent is an inhibitor of the TS-associated gene and reduces a symptom of TS. Alternatively, an increase of the level or activity compared to a normal control level or activity of the gene indicates that said test agent is an enhancer of expression or function of the TS associated gene and reduces a symptom of TS, e.g, TS 347-939.

The invention also provides a kit with a detection reagent which binds to two or more TS nucleic acid sequences or which binds to a gene product encoded by the nucleic acid sequences. Also provided is an array of nucleic acids that binds to two or more TS nucleic acids.

Therapeutic methods include a method of treating or preventing TS in a subject by administering to the subject an antisense composition. The antisense composition reduces the expression of a specific target gene, e.g., the antisense composition contains a nucleotide, which is complementary to a sequence selected from the group consisting of TS 1-346. Another method includes the steps of administering to a subject an short interfering RNA (siRNA) composition. The siRNA composition reduces the expression of a nucleic acid selected from the group consisting of TS 1-346. We demonstrated that PYPAF3 was commonly up-regulated in testicular seminomas and knock down of PYPAF3 transcript by small interference RNA (siRNA) inhibited cell growth of testicular germ cell tumor cells.

In yet another method, treatment or prevention of TS in a subject is carried out by administering to a subject a ribozyme composition. The nucleic acid-specific ribozyme composition reduces the expression of a nucleic acid selected from the group consisting of TS 1-346. Other therapeutic methods include those in which a subject is administered a compound that increases the expression of TS 347-939 or activity of a polypeptide encoded by TS 347-939. Furthermore, TS can be treated by administering a protein encoded by TS 347-939. The protein may be directly administered to the patient or, alternatively, may be expressed in vivo subsequent to being introduced into the patient, for example, by administering an expression vector or host cell carrying the down-regulated marker gene of interest. Suitable mechanisms for in vivo expression of a gene of interest are known in the art.

The invention also includes vaccines and vaccination methods. For example, a method of treating or preventing TS in a subject is carried out by administering to the subject a vaccine containing a polypeptide encoded by a nucleic acid selected from the group consisting of TS 1-346 or an immunologically active fragment such a polypeptide. An immunologically active fragment is a polypeptide that is shorter in length than the full-length naturally-occurring protein and which induces an immune response. For example, an immunologically active fragment at least 8 residues in length and stimulates an immune cell such as a T cell or a B cell. Immune cell stimulation is measured by detecting cell proliferation, elaboration of cytokines (e.g., IL-2), or production of an antibody.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

One advantage of the methods described herein is that the disease is identified prior to detection of overt clinical symptoms. Other features and advantages of the invention will be apparent from the following detailed description, and from the claims.

### BRIEF DESCRIPTION OF THE FIGURES

FIG. 1 depicts photograph of a DNA agarose gel showing expression of representative 28 genes and TUBA examined by semi-quantitative RT-PCR using cDNA prepared from amplified RNA. The first 11 lanes show the expression level of the genes in a different TS patient. The last lane shows the expression level of each gene in testis from a normal individual. Gene symbols are noted for the genes.

FIG. 2A depicts expression of PYPAF3 examined by semi-quantitative RT-PCR in 8 testicular seminoma clinical samples (o. 1, 2, 7, 8, 9, 10, 11 and 13), normal human testis (TES), heart (HER), lung (LUN), liver (LIV), kidney (KID), brain (BRA) and bone marrow (BM). Expression of TUBA3 served as an internal control. FIG. 2B depicts northern analysis with a multiple-tissue blot using PYPAF3 cDNA fragment as a probe.

FIG. 3 depicts sub-cellular localization of myc-tagged PYPAF3 protein. Myc-tagged PYPAF3 protein extracts of COS-7 cells transfected with pcDNA3.1-mycaHis-PYPAF3 plasmids. The transfected cells were stained with mouse anti-myc monoclonal antibody and visualized by FITC-conjugated anti-mouse IgG secondary antibody. Nuclei were counter-stained with DAPI.

FIG. 4 depicts growth-inhibitory effects of small-interference RNAs (siRNAs) designed to reduce expression of PYPAF3 in testicular germ cell tumor line Tera-2. (A) Semi-quantitative RT-PCR showing suppression of endogenous expression of PYPAF3 in testicular germ cell tumor line Tera-2 at two weeks (cultures in selective medium containing neomycin after introduction of siRNAs into testicular germ cell tumor line Tera-2 cells. P2-microgloblin (β2MG) was used as an internal control. (3) Colony-formation assay demonstrating a decrease in the numbers of colonies by knockdown of PYPAF3 (Si1, Si2, Si3, Si4, and Si5) in testicular germ cell tumor line Tera-2 cells at two weeks, compared to psiU6BX-EGFP (siEGFP), psiU6BX-Luciferase (siLuc) as controls. (C) MTT assay of testicular germ cell tumor line Tera-2 cells treated with either psiU6BX-PYPAF3 (Si1, Si2, Si3, Si4, and Si5), psiU6BX-EGFP (siEGFP), psiU6BX-Luciferase (siLuc) by using Cell Counting Kit-8 at one week. These experiments were carried out three times as well.

### DETAILED DESCRIPTION

The present invention is based in part on the discovery of changes in expression patterns of multiple nucleic acid sequences in cells from testis of patients with TS. The differences in gene expression were identified by using a comprehensive cDNA microarray system.

Using a cDNA microarray containing 23,040 genes, comprehensive gene-expression profiles of 13 patients were constructed. Certain genes are expressed at low or high levels in TS patients. In the process candidate molecular markers were selected with the potential of detecting cancer-related proteins in serum or sputum of patients, and some potential targets for development of signal-suppressing strategies in human testicular cancer were discovered.

The differentially expressed genes identified herein are used for diagnostic purposes as markers of TS and as gene targets, the expression of which is altered to treat or alleviate a symptom of TS.

The genes whose expression levels are modulated (i.e., increased or decreased) in TS patients are summarized in Tables 3,4 and are collectively referred to herein as ” TS-associated genes ” TS-associated genes “TS nucleic acids” or “TS polynucleotides” and the corresponding encoded polypeptides are referred to as “TS polypeptides” or “TS proteins.” Unless indicated otherwise, “TS” is meant to refer to any of the sequences disclosed herein. (e.g., TS 1-939). The genes have been previously described and are presented along with a database accession number.

By measuring expression of the various genes in a sample of cells, TS is diagnosed. Similarly, by measuring the expression of these genes in response to various agents, and agents for treating TS can be identified.

The invention involves determining (e.g., measuring) the expression of at least one, and up to all the TS sequences listed in Tables 3,4. Using sequence information provided by the GeneBank™ database entries for the known sequences the TS associated genes are detected and measured using techniques well known to one of ordinary skill in the art. For example, sequences within the sequence database entries corresponding to TS sequences, are used to construct probes for detecting TS RNA sequences in, e.g., northern blot hybridization analyses. Probes include at least 10, 20, 50, 100, 200 nucleotides of a reference sequence. As another example, the sequences can be used to construct primers for specifically amplifying the TS sequences in, e.g, amplification-based detection methods such as reverse-transcription based polymerase chain reaction.

Expression level of one or more of the TS sequences in the test cell population, e.g., a patient derived tissues sample is then compared to expression levels of the some sequences in a reference population. The reference cell population includes one or more cells for which the compared parameter is known, i.e., TS cells or non-TS cells.

Whether or not a pattern of gene expression in the test cell population compared to the reference cell population indicates TS or a predisposition thereto depends upon the composition of the reference cell population. For example, if the reference cell population is composed of non-TS cells, a similar gene expression pattern in the test cell population and reference cell population indicates the test cell population is non-TS. Conversely, if the reference cell population is made up of TS cells, a similar gene expression profile between the test cell population and the reference cell population indicates that the test cell population includes TS cells.

A level of expression of a TS marker gene in a test cell population is considered altered in levels of expression if its expression level varies from the reference cell population by more than 1.0, 1.5, 2.0, 5.0, 10.0 or more fold from the expression level of the corresponding TS sequence in the reference cell population.

Differential gene expression between a test cell population and a reference cell population is normalized to a control nucleic acid, e.g. a housekeeping gene. For example, a control nucleic acid is one which is known not to differ depending on the endometriotic or non-endometriotic state of the cell. Expression levels of the control nucleic acid in the test and reference nucleic acid can be used to normalize signal levels in the compared populations. Control genes include β-actin, glyceraldehyde 3-phosphate dehydrogenase or ribosomal protein P1.

The test cell population is compared to multiple reference cell populations. Each of the multiple reference populations may differ in the known parameter. Thus, a test cell population may be compared to a second reference cell population known to contain, e.g., TS cells, as well as a second reference population known-to contain, e.g., non-TS cells (normal cells). The test cell is included in a tissue type or cell sample from a subject known to contain, or to be suspected of containing, TS cells.

The test cell is obtained from a bodily tissue or a bodily fluid, e.g., biological fluid (such as blood or urine). For example, the test cell is purified from a tissue. Preferably, the test cell population comprises an epithelial cell. The epithelial cell is from tissue known to be or suspected to be a TS.

Cells in the reference cell population are derived from a tissue type as similar to test cell. Optionally, the reference cell population is a cell line, e.g. a TS cell line (positive control) or a normal non-TS cell line (negative control). Alternatively, the control cell population is derived from a database of molecular information derived from cells for which the assayed parameter or condition is known.

The subject is preferably a mammal. The mammal can be, e.g., a human, non-human primate, mouse, rat, dog, cat, horse, or cow.

Expression of the genes disclosed herein is determined at the protein or nucleic acid level using methods known in the art. For example, Northern hybridization analysis using probes which specifically recognize one or more of these sequences can be used to determine gene expression. Alternatively, expression is measured using reverse-transcription-based PCR assays, e.g., using primers specific for the differentially expressed sequences. Expression is also determined at the protein level, i.e., by measuring the levels of polypeptides encoded by the gene products described herein, or biological activity thereof. Such methods are well known in the art and include, e.g., immunoassays based on antibodies to proteins encoded by the genes. The biological activity of the proteins encoded by the genes are also well known.

Diagnosing TS

TS is diagnosed by measuring the level of expression of one or more TS nucleic acid sequences from a test population of cells, (i.e., a patient derived biological sample). Preferably, the test cell population contains an epithelial cell, e.g., a cell obtained from testis tissue. Gene expression is also measured from blood or other bodily fluids such as urine. Other biological samples can be used for measuring the protein level. For example, the protein level in the blood, or serum derived from subject to be diagnosed can be measured by immunoassay or biological assay.

Expression of one or more of TS-associated genes, e.g., TS 1-939 is determined in the test cell or biological sample and compared to the expression of the normal control level. A normal control level is an expression profile of TS-associated genes typically found in a population known not to be suffering from TS. An increase or a decrease of the level of expression in the patient derived tissue sample of the TS associated genes indicates that the subject is suffering from or is at risk of developing TS. For example, an increase in expression of TS 1-346 in the test population compared to the normal control level indicates that the subject is suffering from or is at risk of developing TS. Conversely, a decrease in expression of TS 347-939 in the test population compared to the normal control level indicates that the subject is suffering from or is at risk of developing TS.

When one or more of the TS -associated genes are altered in the test population compared to the normal control level indicates that the subject suffers from or is at risk of developing TS. For example, at least 1%, 5%, 25%, 50%, 60%, 80%, 90% or more of the panel of TS-associated genes (TS 1-346, TS 347-939, or TS 1-939) are altered.

Identifying Agents that Inhibit or Enhance TS-associated Gene Expression

An agent that inhibits the expression or activity of a TS-associated gene is identified by contacting a test cell population expressing a TS associated up-regulated gene with a test agent and determining the expression level of the TS associated gene. A decrease in expression in the presence of the agent compared to the normal control level (or compared to the level in the absence of the test agent) indicates the agent is an inhibitor of a TS associated up-regulated gene and useful to inhibit TS.

Alternatively, an agent that enhances the expression or activity of a TS down-regulated associated gene is identified by contacting a test cell population expressing a TS associated gene with a test agent and determining the expression level or activity of the TS associated down-regulated gene. An increase of expression or activity compared to a normal control expression level or activity of the TS-associated gene indicates that the test agent augments expression or activity of the down-regulated TS associated gene.

The test cell population is any cell expressing the TS-associated genes. For example, the test cell population contains an epithelial cell, such as a cell is or derived from testis. For example, the test cell is an immortalized cell line derived from testicular germ cell tumor. Alternatively, the test cell is a cell, which has been transfected with a TS-associated gene or which has been transfected with a regulatory sequence (e.g. promoter sequence) from a TS-associated gene operably linked to a reporter gene.

Assessing Efficacy of Treatment of TS in a Subject

The differentially expressed TS sequences identified herein also allow for the course of treatment of TS to be monitored. In this method, a test cell population is provided from a subject undergoing treatment for TS. If desired, test cell populations are obtained from the subject at various time points before, during, or after treatment. Expression of one or more of the TS sequences, in the cell population is then determined and compared to a reference cell population which includes cells whose TS state is known. The reference cells have not been exposed to the treatment.

If the reference cell population contains no TS cells, a similarity in expression between TS sequences in the test cell population and the reference cell population indicates that the treatment is efficacious. However, a difference in expression between TS sequences in the test population and a normal control reference cell population indicates the less favorable clinical outcome or prognosis.

By “efficacious” is meant that the treatment leads to a reduction in expression of a pathologically up-regulated gene, increase in expression of a pathologically down-regulated gene or a decrease in size, prevalence, or metastatic potential of testicular tumors in a subject. When treatment is applied prophylactically, “efficacious” means that the treatment retards or prevents TS from forming or retards, prevents, or alleviates a symptom of clinical TS. Assesment of testicular tumors are made using standard clinical protocols.

Efficaciousness is determined in association with any known method for diagnosing or treating TS. TS is diagnosed for example, by identifying symptomatic anomalies, e.g., painless enlargement of the testis.

Selecting a Therapeutic Agent for Treating TS that is Appropriate for a Particular Individual

Differences in the genetic makeup of individuals can result in differences in their relative abilities to metabolize various drugs. An agent that is metabolized in a subject to act as an anti-TS agent can manifest itself by inducing a change in gene expression pattern in the subject's cells from that characteristic of an TS state to a gene expression pattern characteristic of a non-TS state. Accordingly, the differentially expressed TS sequences disclosed herein allow for a putative therapeutic or prophylactic inhibitor of TS to be tested in a test cell population from a selected subject in order to determine if the agent is a suitable inhibitor of TS in the subject.

To identify an inhibitor or enhancer of TS, that is appropriate for a specific subject, a test cell population from the subject is exposed to a therapeutic agent, and the expression of one or more of TS 1-939 sequences is determined.

The test cell population contains a TS cell expressing a TS associated gene. Preferably, the test cell is an epithelial cell. For example a test cell population is incubated in the presence of a candidate agent and the pattern of gene expression of the test sample is measured and compared to one or more reference profiles, e.g., a TS reference expression profile or a non-TS reference expression profile.

A decrease in expression of one or more of the sequences TS 1-346 or an increase in expression of one or more of the sequences TS 347-939 in a test cell population relative to a reference cell population containing TS is indicative that the agent is therapeutic.

The test agent can be any compound or composition. For example, the test agents are immunomodulatory agents.

Screening Assays for Identifying Therapeutic Agents

The differentially expressed sequences disclosed herein can also be used to identify candidate therapeutic agents for treating a TS. The method is based on screening a candidate therapeutic agent to determine if it converts an expression profile of TS 1-939 sequences characteristic of a TS state to a pattern indicative of a non-TS state.

In the method, a cell is exposed to a test agent or a combination of test agents (sequentially or consequentially) and the expression of one or more TS 1-939 sequences in the cell is measured. The expression profile of the TS sequences in the test population is compared to expression level of the TS sequences in a reference cell population that is not exposed to the test agent.

An agent effective in stimulating expression of under-expressed genes, or in suppressing expression of over-expressed genes is deemed to lead to a clinical benefit such compounds are further tested for the ability to prevent endometrial cyst growth, e.g., endometrial glands and/or stroma, in animals or test subjects.

In a further embodiment, the present invention provides methods for screening candidate agents which are potential targets in the treatment of TS. As discussed in detail above, by controlling the expression levels or activities of marker genes, one can control the onset and progression of TS. Thus, candidate agents, which are potential targets in the treatment of TS, can be identified through screenings that use the expression levels and activities of marker genes as indices. In the context of the present invention, such screening may comprise, for example, the following steps:

a) contacting a test compound with a polypeptide encoded by TS 1-939;

b) detecting the binding activity between the polypeptide and the test compound; and

c) selecting a compound that binds to the polypeptide

Alternatively, the screening method of the present invention may comprise the following steps:
a) contacting a candidate compound with a cell expressing one or more marker genes, wherein the one or more marker genes is selected from the group consisting of TS 1-939; and
b) selecting a compound that reduces the expression level of one or more marker genes selected from the group consisting of TS 1-346, or elevates the expression level of one or more marker genes selected from the group consisting of TS 347-939.
   Cells expressing a marker gene include, for example, cell lines established from TS; such cells can be used for the above screening of the present invention.

Alternatively, the screening method of the present invention may comprise the following steps:
a) contacting a test compound with a polypeptide encoded by selected from the group consisting of TS 1-939;
b) detecting the biological activity of the polypeptide of step (a); and
c) selecting a compound that suppresses the biological activity of the polypeptide encoded by TS 1-346 in comparison with the biological activity detected in the absence of the test compound, or enhances the the biological activity of the polypeptide encoded by TS 347-939 in comparison with the biological activity detected in the absence of the test compound.
   A protein required for the screening can be obtained as a recombinant protein using the nucleotide sequence of the marker gene. Based on the information of the marker gene, one skilled in the art can select any biological activity of the protein as an index for screening and a measurement method based on the selected biological activity.

Alternatively, the screening method of the present invention may comprise the following steps:
a) contacting a candidate compound with a cell into which a vector comprising the transcriptional regulatory region of one or more marker genes and a reporter gene that is expressed under the control of the transcriptional regulatory region has been introduced, wherein the one or more marker genes are selected from the group consisting of TS 1-939
b) measuring the activity of said reporter gene; and
c) selecting a compound that reduces the expression level of said reporter gene when said marker gene is an up-regulated marker gene selected from the group consisting of TS 1-346 or that enhances the expression level of said reporter gene when said marker gene is a down-regulated marker gene selected from the group consisting of TS 347-939, as compared to a control.

Suitable reporter genes and host cells are well known in the art. The reporter construct required for the screening can be prepared by using the transcriptional regulatory region of a marker gene. When the transcriptional regulatory region of a marker gene has been known to those skilled in the art, a reporter construct can be prepared by using the previous sequence information. When the transcriptional regulatory region of a marker gene remains unidentified, a nucleotide segment containing the transcriptional regulatory region can be isolated from a genome library based on the nucleotide sequence information of the marker gene.

The compound isolated by the screening is a candidate for drugs that inhibit the activity of the protein encoded by marker genes and can be applied to the treatment or prevention of TS.

Moreover, compound in which a part of the structure of the compound inhibiting the activity of proteins encoded by marker genes is converted by addition, deletion and/or replacement are also included in the compounds obtainable by the screening method of the present invention.

When administrating the compound isolated by-the method of the invention as a pharmaceutical for humans and other mammals, such as mice, rats, guinea-pigs, rabbits, cats, dogs, sheep, pigs, cattle, monkeys, baboons, and chimpanzees, the isolated compound can be directly administered or can be formulated into a dosage form using known pharmaceutical preparation methods. For example, according to the need, the drugs can be taken orally, as sugar-coated tablets, capsules, elixirs and microcapsules, or non-orally, in the form of injections of sterile solutions or suspensions with water or any other pharmaceutically acceptable liquid. For example, the compounds can be mixed with pharmaceutically acceptable carriers or media, specifically, sterilized water, physiological saline, plant-oils, emulsifiers, suspending agents, surfactants, stabilizers, flavoring agents, excipients, vehicles, preservatives, binders, and such, in a unit dose form required for generally accepted drug implementation. The amount of active ingredients in these preparations makes a suitable dosage within the indicated range acquirable.

Examples of additives that can be mixed to tablets and capsules are, binders such as gelatin, corn starch, tragacanth gum and arabic gum; excipients such as crystalline cellulose; swelling agents such as corn starch, gelatin and alginic acid; lubricants such as magnesium stearate; sweeteners such as sucrose, lactose or saccharin; and flavoring agents such as peppermint, Gaultheria adenothrix oil and cherry. When the unit-dose form is a capsule, a liquid carrier, such as an oil, can also be further included in the above ingredients. Sterile composites for injections can be formulated following normal drug implementations using vehicles such as distilled water used for injections.

Physiological saline, glucose, and other isotonic liquids including adjuvants, such as D-sorbitol, D-mannnose, D-mannitol, and sodium chloride, can be used as aqueous solutions for injections. These can be used in conjunction with suitable solubilizers, such as alcohol, specifically ethanol, polyalcohols such as propylene glycol and polyethylene glycol, non-ionic surfactants, such as Polysorbate 80 (TM) and HCO-50.

Sesame oil or Soy-bean oil can be used as a oleaginous liquid and may be used in conjunction with benzyl benzoate or benzyl alcohol as a solubilizer and may be formulated with a buffer, such as phosphate buffer and sodium acetate buffer; a pain-killer, such as procaine hydrochloride; a stabilizer, such as benzyl alcohol and phenol; and an anti-oxidant. The prepared injection may be filled into a suitable ampule.

Methods well known to one skilled in the art may be used to administer the pharmaceutical composition of the present invention to patients, for example as intraarterial, intravenous, or percutaneous injections and also as intranasal, transbronchial, intramuscular or oral administrations. The dosage and method of administration vary according to the body-weight and age of a patient and the administration method; however, one skilled in the art can routinely select a suitable method of administration. If said compound is encodable by a DNA, the DNA can be inserted into a vector for gene therapy and the vector administered to a patient to perform the therapy. The dosage and method of administration vary according to the body-weight, age, and symptoms of the patient but one skilled in the art can suitably select them.

For example, although the dose of a compound that binds to the protein of the present invention and regulates its activity depends on the symptoms, the dose is about 0.1 mg to about 100 mg per day, preferably about 1.0 mg to about 50 mg per day and more preferably about 1.0 mg to about 20 mg per day, when administered orally to a normal adult (weight 60 kg).

When administering parenterally, in the form of an injection to a normal adult (weight 60 kg), although there are some differences according to the patient, target organ, symptoms and method of administration, it is convenient to intravenously inject a dose of about 0.01 mg to about 30 mg per day, preferably about 0.1 to about 20 mg per day and more preferably about 0.1 to about 10 mg per day. Also, in the case of other animals too, it is possible to administer an amount converted to 60 kgs of body-weight.

Assessing the Prognosis of a Subject with TS

Also provided is a method of assessing the prognosis of a subject with TS by comparing the expression of one or more TS sequences in a test cell population to the expression of the sequences in a reference cell population derived from patients over a spectrum of disease stages. By comparing gene expression of one or more TS sequences in the test cell population and the reference cell population(s), or by comparing the pattern of gene expression over time in test cell populations derived from the subject, the prognosis of the subject can be assessed.

A decrease in expression of one or more of the sequences TS 347-939 compared to a normal control or an increase of expression of one or more of the sequences TS 1-346 compared to a normal control indicates less favorable prognosis. An increase in expression of one or more of the sequences TS 347-939 indicates a more favorable prognosis, and a decrease in expression of sequences TS 1-346 indicates a more favorable prognosis for the subject.

Kits

The invention also includes a TS-detection reagent, e.g., a nucleic acid that specifically binds to or identifies one or more TS nucleic acids such as oligonucleotide sequences, which are complementary to a portion of a TS nucleic acid or antibodies which bind to proteins encoded by a TS nucleic acid. The reagents are packaged together in the form of a kit. The reagents are packaged in separate containers, e.g., a nucleic acid or antibody (either bound to a solid matrix or packaged separately with reagents for binding them to the matrix), a control reagent (positive and/or negative), and/or a detectable label. Instructions (e.g., written, tape, VCR, CD-ROM, etc.) for carrying out the assay are included in the kit. The assay format of the kit is a Northern hybridization or a sandwich ELISA known in the art.

For example, TS detection reagent is immobilized on a solid matrix such as a porous strip to form at least one TS detection site. The measurement or detection region of the porous strip may include a plurality of sites containing a nucleic acid. A test strip may also contain sites for negative and/or positive controls. Alternatively, control sites are located on a separate strip from the test strip. Optionally, the different detection sites may contain different amounts of immobilized nucleic acids, i.e., a higher amount in the first detection site and lesser amounts in subsequent sites. Upon the addition of test sample, the number of sites displaying a detectable signal provides a quantitative indication of the amount of TS present in the sample. The detection sites may be configured in any suitably detectable shape and are typically in the shape of a bar or dot spanning the width of a teststrip.

Alternatively, the kit contains a nucleic acid substrate array comprising one or more nucleic acid sequences. The nucleic acids on the array specifically identify one or more nucleic acid sequences represented by TS 1-939. The expression of 2, 3, 4, 5, 6, 7, 8, 9, 10, 15, 20, 25, 40 or 50 or more of the sequences represented by TS 1-939 are identified by virtue if the level of binding to an array test strip or chip. The substrate array can be on, e.g., a solid substrate, e.g., a “chip” as described in U.S. Pat. No.5,744,305.

Arrays and Pluralities

The invention also includes a nucleic acid substrate array comprising one or more nucleic acid sequences. The nucleic acids on the array specifically correspond to one or more nucleic acid sequences represented by TS 1-939. The level expression of 2, 3, 4, 5, 6, 7, 8, 9, 10, 15, 20, 25, 40 or 50 or more of the sequences represented by TS 1-939 are identified by detecting nucleic acid binding to the array.

The invention also includes an isolated plurality (ie., a mixture if two or more nucleic acids) of nucleic acid sequences. The nucleic acid sequence are in a liquid phase or a solid phase, e.g., immobilized on a solid support such as a nitrocellulose membrane. The plurality includes one or more of the nucleic acid sequences represented by TS 1-939. In various embodiments, the plurality includes 2, 3, 4, 5, 6, 7, 8, 9, 10, 15, 20, 25, 40 or 50 or more of the sequences represented by TS 1-939.

Methods of Inhibiting TS

The invention provides a method for treating or alleviating a symptom of TS in a subject by decreasing expression or activity of TS 1-346 or increasing expression or activity of TS 347-939. Therapeutic compounds are administered prophylactically or therapeutically to subject suffering from at risk of (or susceptible tp) developing TS. Such subjects are identified using standard clinical methods or by detecting an aberrant level of expression or activity of (e.g., TS 1-939). Therapeutic agents include inhibitors of cell cycle regulation, cell proliferation, and protein kinase activity.

The therapeutic method includes increasing the expression, or function, or both of one or m ore gene products of genes whose expression is decreased (under-expressed genes”) in a TS cell relative to normal cells of the same tissue type from which the TS cells are derived. In these methods, the subject is treated with an effective amount of a compound, which increases the amount of one of more of the under-expressed genes in the subject. Administration can be systemic or local. Therapeutic compounds include a polypeptide product of an under-expressed gene, or a biologically active fragment thereof a nucleic acid encoding an under-expressed gene and having expression control elements permitting expression in the TS cells; for example an agent which increases the level of expression of such gene endogenous to the TS cells (i.e., which up-regulates expression of the under-expressed gene or genes). Administration of such compounds counter the effects of aberrantly-under expressed of the gene or genes in the subjects testis cells and improves the clinical condition of the subject.

The method also includes decreasing the expression, or function, or both, of one or more gene products of genes whose expression is aberrantly increased (“over-expressed gene”) in testis cells. Expression is inhibited in any of several ways known in the art. For example, expression is inhibited by administering to the subject a nucleic acid that inhibits, or antagonizes, the expression of the over-expressed gene or genes, e.g., an antisense oligonucleotide or small interfering RNA which disrupts expression of the over-expressed gene or genes.

As noted above, antisense nucleic acids corresponding to the nucleotide sequence of TS 1-346 can be used to reduce the expression level of the TS 1-346. Antisense nucleic acids corresponding to TS 1-346 that are up-regulated in TS are useful for the treatment of TS. Specifically, the antisense nucleic acids of the present invention may act by binding to the TS 1-346 or mRNAs corresponding thereto, thereby inhibiting the transcription or translation of the genes, promoting the degradation of the mRNAs, and/or inhibiting the expression of proteins encoded by the TS 1-346, finally inhibiting the function of the proteins. The term “antisense nucleic acids” as used herein encompasses both nucleotides that are entirely complementary to the target sequence and those having a mismatch of one or more nucleotides, so long as the antisense nucleic acids can specifically hybridize to the target sequences. For example, the antisense nucleic acids of the present invention include polynucleotides that have a homology of at least 70% or higher, preferably at 80% or higher, more preferably 90% or higher, even more preferably 95% or higher over a span of at least 15 continuous nucleotides. Algorithms known in the art can be used to determine the homology.

The antisense nucleic acid derivatives of the present invention act on cells producing the proteins encoded by marker genes by binding to the DNAs or niRNAs encoding the proteins, inhibiting their transcription or translation, promoting the degradation of the mRNAs, and inhibiting the expression of the proteins, thereby resulting in the inhibition of the protein function.

An antisense nucleic acid derivative of the present invention can be made into an external preparation, such as a liniment or a poultice, by mixing with a suitable base material which is inactive against the derivative.

Also, as needed, the derivatives can be formulated into tablets, powders, granules, capsules, liposome capsules, injections, solutions, nose-drops and freeze-drying agents by adding excipients, isotonic agents, solubilizers, stabilizers, preservatives, pain-killers, and such. These can be prepared by following known methods.

The antisense nucleic acids derivative is given to the patient by directly applying onto the ailing site or by injecting into a blood vessel so that it will reach the site of ailment. An antisense-mounting medium can also be used to increase durability and membrane-permeability. Examples are, liposomes, poly-L-lysine, lipids, cholesterol, lipofectin or derivatives of these.

The dosage of the antisense nucleic acid derivative of the present invention can be adjusted suitably according to the patient's condition and used in desired amounts. For example, a dose range of 0.1 to 100 mg/kg, preferably 0.1 to 50 mg/kg can be administered.

The antisense nucleic acids of the invention inhibit the expression of the protein of the invention and is thereby useful for suppressing the biological activity of a protein of the invention. Also, expression-inhibitors, comprising the antisense nucleic acids of the invention, are useful since they can inhibit the biological activity of a protein of the invention.

The antisense nucleic acids of present invention include modified oligonucleotides. For example, thioated nucleotides may be used to confer nuclease resistance to an oligonucleotide.

Also, a siRNA against marker gene can be used to reduce the expression level of the marker gene. By the term “siRNA” is meant a double stranded RNA molecule which prevents translation of a target mRNA. Standard techniques of introducing siRNA into the cell are used, including those in which DNA is a template from which RNA is transcribed. In the context of the present invention, the siRNA comprises a sense nucleic acid sequence and an anti-sense nucleic acid sequence against an upregulated marker gene, such as TS 1-346. The siRNA is constructed such that a single transcript has both the sense and complementary antisense sequences from the target gene, e.g., a hairpin.

The method is used to alter the expression in a cell of an upregulated, e.g., as a result of malignant transformation of the cells. Binding of the siRNA to a transcript corresponding to one of the TS 1-346 in the target cell results in a reduction in the protein production by the cell. The length of the oligonucleotide is at least 10 nucleotides and may be as long as the naturally-occurring the transcript. Preferably, the oligonucleotide is 19-25 nucleotides in length. Most preferably, the oligonucleotide is less than 75, 50, 25 nucleotides in length. For example, siRNAs for PYPAF3 comprising nucleotide sequence of SEQ ID NO: 85 or 86 as the target sequence inhibit the cell proliferation of TS.

The nucleotide sequence of the siRNAs were designed using a siRNA design computer program available from the Ambion website (http://www.ambion.com/techlib/misc/ siRNA_finder.html). The computer program selects nucleotide sequences for siRNA synthesis based on the following protocol.

Selection of siRNA Target Sites:
1. Beginning with the AUG start codon of the object transcript, scan downstream for AA dinucleotide sequences. Record the occurrence of each AA and the 3′ adjacent 19 nucleotides as potential siRNA target sites. Tuschl, et al. recommend against designing siRNA to the 5′ and 3′ untranslated regions (UTRs) and regions near the start codon (within 75 bases) as these may be richer in regulatory protein binding sites. UTR-binding proteins and/or translation initiation complexes may interfere with the binding of the siRNA endonuclease complex.
2. Compare the potential target sites to the human genome database and eliminate from consideration any target sequences with significant homology to other coding sequences.

The homology search can be performed using BLAST, which can be found on the NCBI server at: www.ncbi.nlm.nih.gov/BLAST/
3. Select qualifying target sequences for synthesis. At Ambion, preferably several target sequences can be selected along the length of the gene for evaluation

The antisense oligonucleotide or siRNA of the invention inhibit the expression of the polypeptide of the invention and is thereby useful for suppressing the biological activity of the polypeptide of the invention. Also, expression-inhibitors, comprising the antisense oligonucleotide or siRNA of the invention, are useful in the point that they can inhibit the biological activity of the polypeptide of the invention. Therefore, a composition comprising the antisense oligonucleotide or siRNA of the present invention are useful in treating a TS.

Alternatively, function of one or more gene products of the over-expressed genes is inhibited by administering a compound that binds to or otherwise inhibits the function of the gene products. For example, the compound is an antibody which binds to the over-expressed gene product or gene products.

The present invention refers to the use of antibodies, particularly antibodies against a protein encoded by an up-regulated marker gene, or a fragment of the antibody. As used herein, the term “antibody” refers to an immunoglobulin molecule having a specific structure, that interacts (i.e., binds) only with the antigen that was used for synthesizing the antibody (i.e., the up-regulated marker gene product) or with an antigen closely related to it. Furthermore, an antibody may be a fragment of an antibody or a modified antibody, so long as it binds to one or more of the proteins encoded by the marker genes. For instance, the antibody fragment may be Fab, F(ab′)₂, Fv, or single chain Fv (scFv), in which Fv fragments from H and L chains are ligated by an appropriate linker (Huston J. S. et al. Proc. Natl. Acad. Sci. U.S.A. 85:5879-5883 (1988)). More specifically, an antibody fragment may be generated by treating an antibody with an enzyme, such as papain or pepsin. Alternatively, a gene encoding the antibody fragment may be constructed, inserted into an expression vector, and expressed in an appropriate host cell (see, for example, Co M. S. et al. J. Immunol. 152:2968-2976 (1994); Better M. and Horwitz A. H. Methods Enzymol. 178:476-496 (1989); Pluckthun A. and Skerra A. Methods Enzymol. 178:497-515 (1989); Lamoyi E. Methods Enzymol. 121:652-663 (1986); Rousseaux J. et al. Methods Enzymol. 121:663-669 (1986); Bird R. E. and Walker B. W. Trends Biotechnol. 9:132-137 (1991)).

An antibody may be modified by conjugation with a variety of molecules, such as polyethylene glycol (PEG). The present invention provides such modified antibodies. The modified antibody can be obtained by chemically modifying an antibody. These modification methods are conventional in the field.

Alternatively, an antibody may be obtained as a chimeric antibody, between a variable region derived from a nonhuman antibody and a constant region derived from a human antibody, or as a humanized antibody, comprising the complementarity determining region (CDR) derived from a nonhuman antibody, the frame work region (FR) derived from a human antibody, and the constant region. Such antibodies can be prepared by using known technologies.

Cancer therapies directed at specific molecular alterations that occur in cancer cells have been validated through clinical development and regulatory approval of anti-cancer drugs such as trastuzumab (Herceptin) for the treatment of advanced breast cancer, imatinib methylate (Gleevec) for chronic myeloid leukemia, gefitinib (Iressa) for non-small cell lung cancer (NSCLC), and rituximab (anti-CD20 mAb) for B-cell lymphoma and mantle cell lymphoma (Ciardiello F, Tortora G. A novel approach in the treatment of cancer: targeting the epidermal growth factor receptor. Clin Cancer Res. 2001 October;7(10):2958-70. Review.; Slamon D J, Leyland-Jones B, Shak S, Fuchs H, Paton V, Bajamonde A, Fleming T, Eiermann W, Wolter J, Pegram M, Baselga J, Norton L. Use of chemotherapy plus a monoclonal antibody against HER2 for metastatic breast cancer that overexpresses HER2. N Engl J Med. 15 Mar 2001;344(11):783-92.; Rehwald U, Schulz H, Reiser M, Sieber M, Staak J O, Morschhauser F, Driessen C, Rudiger T, Muller-Hermelink K, Diehl V, Engert A. Treatment of relapsed CD20+ Hodgkin lymphoma with the monoclonal antibody rituximab is effective and well tolerated: results of a phase 2 trial of the German Hodgkin Lymphoma Study Group. Blood. 15 Jan. 2003;101(2):420424.; Fang G, Kim C N, Perkins C L, Ramadevi N, Winton E, Wittmann S and Bhalla K N. (2000). Blood, 96, 2246-2253.). These drugs are clinically effective and better tolerated than traditional anti-cancer agents because they target only transformed cells. Hence, such drugs not only improve survival and quality of life for cancer patients, but also validate the concept of molecularly targeted cancer therapy. Furthermore, targeted drugs can enhance the efficacy of standard chemotherapy when used in combination with it (Gianni L. (2002). Oncology, 63 Suppl 1, 47-56.; Klejman A, Rushen L, Morrione A, Slupianek A and Skorski T. (2002). Oncogene, 21, 5868-5876.). Therefore, future cancer treatments will probably involve combining conventional drugs with target-specific agents aimed at different characteristics of tumor cells such as angiogenesis and invasiveness.

These modulatory methods are performed ex vivo or in vitro (e.g., by culturing the cell with the agent) or, alternatively, in vivo (e.g., by administering the agent to a subject). The method involves administering a protein or combination of proteins or a nucleic acid molecule or combination of nucleic acid, molecules as therapy to counteract aberrant expression or activity of the differentially expressed genes.

Diseases and disorders that are characterized by increased (relative to a subject not suffering from the disease or disorder) levels or biological activity of the genes may be treated with therapeutics that antagonize (i.e., reduce or inhibit) activity of the over-expressed gene or genes. Therapeutics that antagonize activity are administered therapeutically or prophylactically.

Therapeutics that may be utilized include, e.g., (i) a polypeptide, or analogs, derivatives, fragments or homologs thereof of the underexpressed sequence or sequences; (ii) antibodies to the overexpressed sequence or sequences; (iii) nucleic acids encoding the underexpressed sequence or sequences; (iv) antisense nucleic acids or nucleic acids that are “dysfunctional” (ie., due to a heterologous insertion within the coding sequences of one or more overexpressed sequences); (v) small interfering RNA (siRNA); or (vi) modulators (i.e., inhibitors, agonists and antagonists that alter the interaction between an over/underexpressed polypeptide and its binding partner. The dysfunctional antisense molecules are utilized to “knockout” endogenous function of a polypeptide by homologous recombination (see, e.g., Capecchi, Science 244: 1288-1292 1989).

Diseases and disorders that are characterized by decreased (relative to a subject not suffering from the disease or disorder) levels or biological activity may be treated with therapeutics that increase (i.e., are agonists to) activity. Therapeutics that up-regulate activity may be administered in a therapeutic or prophylactic manner. Therapeutics that may be utilized include, but are not limited to, a polypeptide (or analogs, derivatives, fragments or homologs thereof) or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of a gene whose expression is altered). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, etc.).

Prophylactic administration occurs prior to the manifestation of overt clinical symptoms of disease, such that a disease or disorder is prevented or, alternatively, delayed in its progression.

Therapeutic methods include contacting a cell with an agent that modulates one or more of the activities of the gene products of the differentially expressed genes. An agent that modulates protein activity includes a nucleic acid or a protein, a naturally-occurring cognate ligand of these proteins, a peptide, a peptidomimetic, or other small molecule. For example, the agent stimulates one or more protein activities of one or more of a differentially under-expressed gene.

The present invention also relates to a method of treating or preventing TS in a subject comprising administering to said subject a vaccine comprising a polypeptide encoded by a nucleic acid selected from the group consisting of TS 1-346 or an immunologically active fragment of said polypeptide, or a polynucleotide encoding the polypeptide or the fragment thereof. An administration of the polypeptide induce an anti-tumor immunity in a subject. To inducing anti-tumor immunity, a polypeptide encoded by a nucleic acid selected from the group consisting of TS 1-346 or an immunologically active fragment of said polypeptide, or a polynucleotide encoding the polypeptide is administered. The polypeptide or the immunologically active fragments thereof are useful as vaccines against TS. In some cases the proteins or fragments thereof may be administered in a form bound to the T cell recepor (TCR) or presented by an antigen presenting cell (APC), such as macrophage, dendritic cell (DC), or B-cells. Due to the strong antigen presenting ability of DC, the use of DC is most preferable among the APCs.

In the present invention, vaccine against TS refers to a substance that has the function to induce anti-tumor immunity upon inoculation into animals. According to the present invention, polypeptides encoded by TS 1-346 or fragments thereof were suggested to be HLA-A24 or HLA-A*0201 restricted epitopes peptides that may induce potent and specific immune response against TS cells expressing TS 1-346. Thus, the present invention also encompasses method of inducing anti-tumor immunity using the polypeptides. In general, anti-tumor immunity includes immune responses such as follows:
induction of cytotoxic lymphocytes against tumors,
induction of antibodies that recognize tumors, and
induction of anti-tumor cytokine production.

Therefore, when a certain protein induces any one of these immune responses upon inoculation into an animal, the protein is decided to have anti-tumor immunity inducing effect. The induction of the anti-tumor immunity by a protein can be detected by observing in vivo or in vitro the response of the immune system in the host against the protein.

For example, a method for detecting the induction of cytotoxic T lymphocytes is well known. A foreign substance that enters the living body is presented to T cells and B cells by the action of antigen presenting cells (APCs). T cells that respond to the antigen presented by APC in antigen specific manner differentiate into cytotoxic T cells (or cytotoxic T lymphocytes; CTLs) due to stimulation by the antigen, and then proliferate (this is referred to as activation of T cells). Therefore, CTL induction by a certain peptide can be evaluated by presenting the peptide to T cell by APC, and detecting the induction of CTL. Furthermore, APC has the effect of activating CD4+ T cells, CD8+ T cells, macrophages, eosinophils, and NK cells. Since CD4+ T cells and CD8+ T cells are also important in anti-tumor immunity, the anti-tumor immunity inducing action of the peptide can be evaluated using the activation effect of these cells as indicators.

A method for evaluating the inducing action of CTL using dendritic cells (DCs) as APC is well known in the art. DC is a representative APC having the strongest CTL inducing action among APCs. In this method, the test polypeptide is initially contacted with DC, and then this DC is contacted with T cells. Detection of T cells having cytotoxic effects against the cells of interest after the contact with DC shows that the test polypeptide has an activity of inducing the cytotoxic T cells. Activity of CTL against tumors can be detected, for example, using the lysis of ⁵¹Cr-labeled tumor cells as the indicator. Alternatively, the method of evaluating the degree of tumor cell damage using ³H-thymidine uptake activity or LDH (lactose dehydrogenase)-release release as the indicator is also well known.

Apart from DC, peripheral blood mononuclear cells (PBMCs) may also be used as the APC. The induction of CTL is reported that the it can be enhanced by culturing PBMC in the presence of GM-CSF and IL-4. Similarly, CTL has been shown to be induced by culturing PBMC in the presence of keyhole limpet hemocyanin (KLH) and IL-7.

The test polypeptides confirmed to possess CTL inducing activity by these methods are polypeptides having DC activation effect and subsequent CTL inducing activity. Therefore, polypeptides that induce CTL against tumor cells are useful as vaccines against tumors. Furthermore, APC that acquired the ability to induce CTL against tumors by contacting with the polypeptides are useful as vaccines against tumors. Furthermore, CTL that acquired cytotoxicity due to presentation of the polypeptide antigens by APC can be also used as vaccines against tumors. Such therapeutic methods for tumors using anti-tumor immunity due to APC and CTL are referred to as cellular immunotherapy.

Generally, when using a polypeptide for cellular immunotherapy, efficiency of the CTL-induction is known to increase by combining a plurality of polypeptides having different structures and contacting them with DC. Therefore, when stimulating DC with protein fragments, it is advantageous to use a mixture of multiple types of fragments.

Alternatively, the induction of anti-tumor immunity by a polypeptide can be confirmed by observing the induction of antibody production against tumors. For example, when antibodies against a polypeptide are induced in a laboratory animal immunized with the polypeptide, and when growth of tumor cells is suppressed by those antibodies, the polypeptide can be determined to have an ability to induce anti-tumor immunity.

Anti-tumor immunity is induced by administering the vaccine of this invention, and the induction of anti-tumor immunity enables treatment and prevention of TS. Therapy against cancer or prevention of the onset of cancer includes any of the steps, such as inhibition of the growth of cancerous cells, involution of cancer, and suppression of occurrence of cancer. Decrease in mortality of individuals having cancer, decrease of tumor markers in the blood, alleviation of detectable symptoms accompanying cancer, and such are also included in the therapy or prevention of cancer. Such therapeutic and preventive effects are preferably statistically significant. For example, in observation, at a significance level of 5% or less, wherein the therapeutic or preventive effect of a vaccine against cell proliferative diseases is compared to a control without vaccine administration. For example, Student's t-test, the Mann-Whitney U-test, or ANOVA may be used for statistical analyses.

The above-mentioned protein having immunological activity or a vector encoding the protein may be combined with an adjuvant. An adjuvant refers to a compound that enhances the immune response against the protein when administered together (or successively) with the protein having immunological activity. Examples of adjuvants include cholera toxin, salmonella toxin, alum, and such, but are not limited thereto. Furthermore, the vaccine of this invention may be combined appropriately with a pharmaceutically acceptable carrier. Examples of such carriers are sterilized water, physiological saline, phosphate buffer, culture fluid, and such. Furthermore, the vaccine may contain as necessary, stabilizers, suspensions, preservatives, surfactants, and such. The vaccine is administered systemically or locally. Vaccine administration may be performed by single administration, or boosted by multiple administrations.

When using APC or CTL as the vaccine of this invention, tumors can be treated or prevented, for example, by the ex vivo method. More specifically, PBMCs of the subject receiving treatment or prevention are collected, the cells are contacted with the polypeptide ex vivo, and following the induction of APC or CTL, the cells may be administered to the subject. APC can be also induced by introducing a vector encoding the polypeptide into PBMCs ex vivo. APC or CTL induced in vitro can be cloned prior to administration. By cloning and growing cells having high activity of damaging target cells, cellular immunotherapy can be performed more effectively. Furthermore, APC and CTL isolated in this manner may be used for cellular immunotherapy not only against individuals from whom the cells are derived, but also against similar types of tumors from other individuals.

Furthermore, a pharmaceutical composition for treating or preventing a cell proliferative disease, such as cancer, comprising a pharmaceutically effective amount of the polypeptide of the present invention is provided. The pharmaceutical composition may be used for raising anti tumor inunity.

Pharmaceutical Compositions for Inhibiting TS

Pharmaceutical formulations include those suitable for oral, rectal, nasal, topical (including buccal and sub-lingual), vaginal or parenteral (including intramuscular, sub-cutaneous and intravenous) administration, or for administration by inhalation or insufflation. Preferably, administration is intravenous. The formulations are optionally packaged in discrete dosage units.

Pharmaceutical formulations suitable for oral administration include capsules, cachets or tablets, each containing a predetermined amount of the active ingredient. Formulations also include powders, granules or solutions, suspensions or emulsions. The active ingredient is optionally administered as a bolus electuary or paste. Tablets and capsules for oral administration may contain conventional excipients such as binding agents, fillers, lubricants, disintegrant or wetting agents. A tablet may be made by compression or molding, optionally with one or more formulational ingredients. Compressed tablets may be prepared by compressing in a suitable machine the active ingredients in a free-flowing form such as a powder or granules, optionally mixed with a binder, lubricant, inert diluent, lubricating, surface active or dispersing agent. Molded tablets may be made by molding in a suitable machine a mixture of the powdered compound moistened with an inert liquid diluent. The tablets may be coated according to methods well known in the art. Oral fluid preparations may be in the form of, for example, aqueous or oily suspensions, solutions, emulsions, syrups or elixirs, or may be presented as a dry product for constitution with water or other suitable vehicle before use. Such liquid preparations may contain conventional additives such as suspending agents, emulsifying agents, non-aqueous vehicles (which may include edible oils), or preservatives. The tablets may optionally be formulated so as to provide slow or controlled release of the active ingredient therein. A package of tablets may contain one tablet to be taken on ech of the month.

Formulations for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit dose or multi-dose containers, for example sealed ampoules and vials, and may be stored in a freeze-dried (lyophilized) condition requiring only the addition of the sterile liquid carrier, for example, saline, water-for-injection, immediately prior to use. Alternatively, the formulations may be presented for continuous infusion. Extemporaneous injection solutions and suspensions may be prepared from sterile powders, granules and tablets of the kind previously described.

Formulations for rectal administration include suppositories with standard carriers such as cocoa butter or polyethylene glycol. Formulations for topical administration in the mouth, for example buccally or sublingually, include lozenges, which contain the active ingredient in a flavored base such as sucrose and acacia or tragacanth, and pastilles comprising the active ingredient in a base such as gelatin and glycerin or sucrose and acacia. For intra-nasal administration the compounds of the invention may be used as a liquid spray or dispersible powder or in the form of drops. Drops may be formulated with an aqueous or non-aqueous base also comprising one or more dispersing agents, solubilizing agents or suspending agents.

For administration by inhalation the compounds are conveniently delivered from an insufflator, nebulizer, pressurized packs or other convenient means of delivering an aerosol spray. Pressurized packs may comprise a suitable propellant such as dichlorodifluoromethane, trichlorofluoromethane, dichiorotetrafluoroethane, carbon dioxide or other suitable gas. In the case of a pressurized aerosol, the dosage unit may be determined by providing a valve to deliver a metered amount.

Alternatively, for administration by inhalation or insufflation, the compounds may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflators.

Other formulations include implantable devices and adhesive patches; which release a therapeutic agent.

When desired, the above described formulations, adapted to give sustained release of the active ingredient, may be employed. The pharmaceutical compositions may also contain other active ingredients such as antimicrobial agents, immunosuppressants or preservatives.

It should be understood that in addition to the ingredients particularly mentioned above, the formulations of this invention may include other agents conventional in the art having regard to the type of formulation in question, for example, those suitable for oral administration may include flavoring agents.

Preferred unit dosage formulations are those containing an effective dose, as recited below, or an appropriate fraction thereof, of the active ingredient.

For each of the aforementioned conditions, the-compositions, e.g., polypeptides and organic compounds are administered orally or via injection at a dose of from about 0.1 to about 250 mg/kg per day. The dose range for adult humans is generally from about 5 mg to about 17.5 g/day, preferably about 5 mg to about 10 g/day, and most preferably about 100 mg to about 3 g/day. Tablets or other unit dosage forms of presentation provided in discrete units may conveniently contain an amount which is effective at such dosage or as a multiple of the same, for instance, units containing about 5 mg to about 500 mg, usually from about 100 mg to about 500 mg.

The dose employed will depend upon a number of factors, including the age and sex of the subject, the precise disorder being treated, and its severity. Also the route of administration may vary depending upon the condition and its severity.

The invention will be further described in the following examples, which do not limit the scope of the invention described in the claims. The following examples illustrate the identification and characterization of genes differentially expressed in TS cells.

### EXAMPLE 1

### Preparation of Test Samples

Tissue obtained from diseased tissue (e.g., testis cells from testicular gern cell tumors) and normal tissues were evaluated to identify genes which are differently expressed or a disease state, e.g., TS. The assays were carried out as follows.

Patients, Tissue Samples and Laser-Capture Microdissection (LCM)

TGCT samples were obtained from 13 patients who underwent orchiectomy. Clinical features of these patients are summarized in Table 1. 12 samples diagnosed as seminoma and on sample of both seminoma and yolk sac tumor were used.

All samples were frozen at −80° C. and then embedded in TissueTek OCT medium (Sakura). The frozen specimens were serially sectioned in 8-μm slices with cryostat (Sakura) and were stained with hematoxylin and eosin to define the analyzed regions. Then, seminoma cells were selectively microdissected from each stained tissue with the PixCell II LCM System (Arcturus Engineering) following the manufacture's protocol with several modifications (21).

TABLE 1
Clinical characteristics of thirteen testicular seminomas

| Case No. | Age | Histopathological type | Stage | Outcome |
|---|---|---|---|---|
| 1 | 43 | seminoma | I | survival |
| 2 | 20 | seminoma | I | survival |
| 3 | 34 | seminoma | I | survival |
| 4 | 33 | seminoma | I | survival |
| 5 | 26 | seminoma | I | survival |
| 6 | 34 | seminoma | I | survival |
| 7 | 45 | seminoma | I | survival |
| 8 | 24 | seminoma | I | survival |
| 9 | 44 | seminoma | I | survival |
| 10 | 27 | seminoma | I | survival |
| 11 | 49 | seminoma | I | survival |
| 12 | 42 | seminoma | III B | survival |
| 13 | 33 | seminoma + yolk sac tumor | II B | survival |

Extraction and Purification of RNA and T7-Based RNA Amplification

Total RNAs were extracted from captured cells into 350 μl RLT lysis buffer (QIAGEN). The extracted RNAs were treated for 15 minutes at room temperature with 30 units of DNase I (QIAGEN). All of the DNase I treated RNAs were subjected to T7-based amplification using Ampliscribe T7 Transcription Kit (Epicentre Technologies)(20). Two rounds of amplification yielded 30-238 μg of amplified RNA (aRNA) for each tissue. As the control probe, normal human poly(A)⁺ RNA (Clontech) was amplified two rounds by the T7-based amplification. 2.5 μg aliquots of aRNAs from each cancerous tissue and the control were reverse-transcribed in the presence of Cy5-dCTP and Cy3-dCTP, respectively (22).

Preparation of the cDNA Microarray

A “genome-wide” cDNA microarray system was established containing 23,040 cDNAs selected from the UniGene database (build #131) of the Natlonal Center for Biotechnology Information (NCBI). Briefly, the cDNAs were amplified by RT-PCR using poly (A)⁺ RNA isolated from various human organs as templates; the lengths of the amplicons ranged from 200 to 1,100 bp excluding repetitive or poly(A) sequences. The PCR products were spotted on type 7 glass slides using a Microarray Spotter, Generation III (Amersham Biosciences); 4608 genes were spotted in duplicate on a single slide. Five different sets of slides were prepared (i.e., 23,040 genes total), on each of which the same 52 housekeeping genes and two negative-control genes were spotted as well (23).

Hybridization and Acquisition of Data

Hybridization and washing were performed according to protocols described previously except that all processes were carried out with an Automated Slide Processor (Amersharm Biosciences). The intensity of each hybridization signal was calculated photometrically by the ArrayVision computer program (Amersham Biosciences) and background intensity was subtracted. Normalization of each Cy3- and Cy5-signal intensity was performed using averaged signals from the 52 housekeeping genes. A cut-off value for each expression level was automatically calculated according to background fluctuation. Cy5/Cy3 was calculated as the relative expression ratio. When both Cy3 and Cy5 signal intensities were lower than the cut-off values, expression of the corresponding gene in that sample was assessed as absent according to previous report (23). For other genes the Cy5/Cy3 ratio was calculated using raw data of each sample.

### EXAMPLE 2

### Identification of TS—Associated Genes

When up- or down-regulated genes common to TS were identified, the genes were analyzed according to the following criteria. Initially genes were selected whose relative expression ratio was able to calculate of more than 50% cases and whose expression were up- or down-regulated in more than 70% of cases. Moreover, if the relative expression ratio was able S to calculate of 35 to 50% cases, the genes were also evaluated that all of cases were up- or down-regulated. The relative expression ratio of each gene (Cy5/Cy3 intensity ratio) was classified into one of four categories as follows: (1) up-regulated (expression ratio was more than 5.0); (2) down-regulated (expression ratio less than 0.2); (3) unchanged expression (expression ratio between 0.2 and 5.0); and (4) not expressed (or slight expression but under the cut-off level for detection). These categories were used to detect a set of genes whose changes in expression ratios were common among samples as well as specific to a certain subgroup. To detect candidate genes that were commonly up- or down-regulated in seminoma cells, the overall expression patterns of 23,040 genes were screened to select genes with expression ratios of more than 5.0 or less than 0.2.

Identification of Genes with Clinically Relevant Expression Patterns in TS Cells

To elucidate genetic events underlying development and progression of TGCTs, we analyzed gene expression in clinical materials by means of a genome-wide cDNA microarray. Microarray technology makes it possible to analyze expression of thousands of genes in a single experiment, and to gain new insights into molecular mechanisms of cancer. Such data are expected to contribute to improvement of clinical management and thereby provide a better quality of life for cancer patients.

One group of investigators analyzed gene-expression profiles using a custom-made cDNA microarray of genes located on chromosome 17 (13), because the long arm of chromosome 17 is frequently over-represented in TGCTs. However, only 636 genes on chromosome 17 and 512 genes from elsewhere in the genome were analyzed in that study. To our knowledge ours is the first “genome-wide” cDNA microarray analysis of TGCTs.

We focused especially on TS, using a comprehensive cDNA microarray system containing 23,040 genes to examine populations of seminoma cells purified by LCM. The proportion of cancer cells selected by this procedure was estimated to be nearly 100%, as determined by microscpic visualization.

Three hundred forty-six up-regulated genes whose expression ratio was more than 5.0 were identified (Table 3), whereas 593 down-regulated genes whose expression ratio was less than 0.2 were identified (Table 4). Furthermore, in particular, 213 highly up-regulated genes whose expression ratio was more than 10.0 were identified (data not shown). On the other hand, 376 down-regulated genes whose expression ratio was less than 0.1 were identified (data not shown).

Some of them might represent potential molecular targets for new therapeutic agents, and/or serve as diagnostic tumor markers. The list of genes in Table 3 included CCND2 (1), POV1 (24), PIM2 (25), JUP (26), and MYCN (14), genes already known to be involved in carcinogenesis or cell proliferation of TS. For example CCND2, which regulates the phosphorylation of RB protein and controls the G1-S cell cycle checkpoint, is often highly expressed in TS; disruption of this checkpoint through over-expression of D-type cyclin is one of the major pathways for tumor development in humans (1). POV1, first identified as a gene that was over-expressed in prostate cancers (24), was later shown to be highly expressed in all TS as well as in carcinoma in situ of the testis (13). This gene encodes a membrane-transport protein with 12 transmembrane domains and may transport nutrients and/or metabolites essential to cell growth (27). Therefore, its product might be a potential molecular-target for anti-cancer drugs for treating TS and prostate cancers. PIM2, a proto-oncogene encoding a serine threonine kinase, was previously reported to be highly expressed in hematopoietic stem cells, leukemic and lymphoma cell lines, and TS; its product appears to have a critical role in hematopoiesis and in oncogenic transformation (25). JUP, also known as gamma-catenin, plays an important role in cell adhesion and the Wnt signaling pathway; JUP is regulated by the APC tumor suppressor gene, and its oncogenic activity in colon cancers is thought to be distinct from that of beta-catenin (26). Amplification of the MYCN gene has been observed in a variety of human tumors, most frequently in neuroblastomas, and its over-expression has been documented in both seminomas and non-seminomas (14). Thus, suppression of these oncogenic functions might be a novel approach to treatment of TS. Moreover, these up-regulated elements included significant genes involved in signal transduction pathway, oncogenes, cell cycle, and cell adhesion and cytoskeleton (Table 5).

In addition to genes known to have some involvement in testicular carcinomas, we noted over-expression of other oncogenes including PIM-1, RET and VAV2. PIM-1, encoding a serine/threonine kinase (28), was over-expressed in all of the 11 informative seminomas examined on our microarray. RET was also over-expressed in all of the six informative seminomas. The RET gene encodes a receptor tyrosine kinase, a cell-surface molecule that transduces signals for cell growth and differentiation; germline mutations in the RET gene are responsible for two hereditary cancer syndromes, multiple endocrine neoplasia types 2A and 2B (29). VAV2, a member of the VAV oncogene family, was over-expressed in 11 of the 12 informative seminoma cases tested on our microarray. The VAV protein is associated with cellular transformation and oncogenesis; it seems to either enhance the metastatic properties of transformed cells or serve as an ancillary factor contributing to the transforming activities of oncoproteins such as Ras (30).

On the other hand, our list of down-regulated genes included at least one known tumor suppressor, WT1, whose inactivation causes Wilms tumor and also WAGR syndrome, which is characterized by susceptibility to Wilms tumor, animdia, genitourinary abnormalities, and mental retardation (31). Loss of heterozygosity in the chromosomal region harboring WT1 has been observed frequently in testicular germ cell tumors (32). Furthermore, Wilms tumor 1-associating protein (KIAAO105, WTAP), a WT1-binding partner, was also down-regulated in our study. Since WT1 is related to normal development of the genitourinary system, its product may be one a candidate for involvement in testicular carcinogenesis although its molecular mechanism remains unclear.

Recent achievement of clinical improvements through use of molecular-targeted drugs has underscored the importance of discovering new molecular targets for development of drugs to treat specific cancers. For example, an anti-HER2 monoclonal antibody, trastuzumab, in conjunction with anti-cancer drugs, antagonizes the proto-oncogene receptor HER2/neu and leads to improvement of clinical response and survival of some breast-cancer patients (33). STI-571, a tyrosine kinase inhibitor targeting bcr-abl, is now a first-line drug for treatment of chronic myeloid leukemias (34), and an epidermal growth factor receptor inhibitor, gefitinib, is useful for treatment of non-small cell lung cancers (35). An anti-CD20 monoclonal antibody, rituximab, has improved rates of complete remission and overall survival for patients with B-cell lymphoma or mantle cell lymphoma (36). Hence, the up-regulated gene products which were identified here and are related to cell proliferation may be promising potential targets for designing novel agents for treating TS. In particular, secreted proteins that function in the autocrine cell-growth pathway should be good candidates for development of drugs and could become novel diagnostic markers for this type of cancer.

Eleven of the 13 cases analyzed in this study were classified clinicopathologically to stage I. Hence, genes which were commonly up-regulated or down-regulated on our microarray are likely to be associated with relatively early phases of carcinogenesis. Consequently, our data provide not only new information about cancer-related genes but also a new correlation of known genes with carcinogenesis. Nonetheless, the information described in the paper disclosed a high degree of complexity among alterations in genetic activities during development of TS; the result is a long list of potential therapeutic targets and/or biomarkers for this type of cancer.

TABLE 3
346 genes commomly up-regulated five-fold or more in testicular seminomas.

| TS | Accession | | |
|---|---|---|---|
| Assignment | No. | Symbol | Gene name |
| 1 | AI141839 | ABCD4 | ATP-binding cassette, sub-family D |
| | | | (ALD), member 4 |
| 2 | X02994 | ADA | adenosine deaminase |
| 3 | U41767 | ADAM15 | a disintegrin and metalloproteinase |
| | | | domain 15 (metargidin) |
| 4 | AF024714 | AIM2 | absent in melanoma 2 |
| 5 | H57960 | AK3 | adenylate kinase 3 |
| 6 | U24266 | ALDH4 | aldehyde dehydrogenase 4 |
| | | | (glutamate gamma-semialdehyde |
| | | | dehydrogenase; pyrroline-5- |
| | | | carboxylate dehydrogenase) |
| 7 | AA180314 | ANKRD2 | Ankyrin repeat domain 2 (stretch |
| | | | responsive muscle) |
| 8 | AA910946 | AP1M2 | adaptor-related protein complex 1, |
| | | | mu 2 subunit |
| 9 | AA676726 | APELIN | apelin; peptide ligand for APJ |
| | | | receptor |
| 10 | U79268 | APEX | APEX nuclease (multifunctional |
| | | | DNA repair enzyme) |
| 11 | X00570 | APOC1 | apolipoprotein C-I |
| 12 | L08424 | ASCL1 | achaete-scute complex (Drosophila) |
| | | | homolog-like 1 |
| 13 | D89052 | ATP6F | ATPase, H+ transporting, lysosomal |
| | | | (vacuolar proton pump) 21 kD |
| 14 | AF038195 | BCS1L | BCS1 (yeast homolog)-like |
| 15 | M88714 | BDKRB2 | bradykinin receptor B2 |
| 16 | AF001383 | BIN1 | bridging integrator 1 |
| 17 | W91908 | BRAG | B cell RAG associated protein |
| 18 | R43935 | CACNA1G | calcium channel, voltage-dependent, |
| | | | alpha 1G subunit |
| 19 | U66063 | CAMK2G | calcium/calmodulin-dependent |
| | | | protein kinase (CaM kinase) II |
| | | | gamma |
| 20 | AA682870 | CCND2 | cyclin D2 |
| 21 | U45983 | CCR8 | chemokine (C—C motif) receptor 8 |
| 22 | M16445 | CD2 | CD2 antigen (p50), sheep red blood |
| | | | cell receptor |
| 23 | AA083656 | CD37 | CD37 antigen |
| 24 | M37033 | CD53 | CD53 antigen |
| 25 | M81934 | CDC25B | cell division cycle 25B |
| 26 | X63629 | CDH3 | cadherin 3, type 1, P-cadherin |
| | | | (placental) |
| 27 | M16965 | CDR1 | cerebellar degeneration-related |
| | | | protein (34 kD) |
| 28 | U51095 | CDX1 | caudal type homeo box transcription |
| | | | factor 1 |
| 29 | AA319695 | CEBPD | CCAAT/enhancer binding protein |
| | | | (C/EBP), delta |
| 30 | U14518 | CENPA | centromere protein A (17 kD) |
| 31 | U58514 | CHI3L2 | chitinase 3-like 2 |
| 32 | X14830 | CHRNB1 | cholinergic receptor, nicotinic, beta |
| | | | polypeptide 1 (muscle) |
| 33 | AC002115 | COX6B | cytochrome c oxidase subunit VIb |
| 34 | X59932 | CSK | c-src tyrosine kinase |
| 35 | AW167729 | CTSC | cathepsin C |
| 36 | AA579959 | CYP2S1 | cytochrome P540 family member |
| | | | predicted from ESTs |
| 37 | N20321 | D19S1177E | DNA segment on chromosome 19 |
| | | | (unique) 1177 expressed sequence |
| 38 | U79775 | D21S2056E | DNA segment on chromosome 21 |
| | | | (unique) 2056 expressed sequence |
| 39 | AI092999 | D2S448 | Melanoma associated gene |
| 40 | Z29093 | DDR1 | discoidin domain receptor family, |
| | | | member 1 |
| 41 | U49785 | DDT | D-dopachrome tautomerase |
| 42 | T78186 | DNMT3A | DNA (cytosine-5-)- |
| | | | methyltransferase 3 alpha |
| 43 | D78011 | DPYS | dihydropyrimidinase |
| 44 | U88047 | DRIL1 | dead ringer (Drosophila)-like 1 |
| 45 | AA128470 | DSP | desmoplakin (DPI, DPII) |
| 46 | X92896 | DXS9879E | DNA segment on chromosome X |
| | | | (unique) 9879 expressed sequence |
| 47 | AA233853 | E1B-AP5 | E1B-55 kDa-associated protein 5 |
| 48 | S49592 | E2F1 | E2F transcription factor 1 |
| 49 | AA422074 | ENO2 | Enolase 2, (gamma, neuronal) |
| 50 | M57736 | ENPP1 | ectonucleotide pyrophosphatase/phosphodiesterase1 |
| 51 | U07695 | EPHB4 | EphB4 |
| 52 | U15655 | ERF | Ets2 repressor factor |
| 53 | D12765 | ETV4 | ets variant gene 4 (E1A enhancer- |
| | | | binding protein, E1AF) |
| 54 | X86779 | FASTK | Fas-activated serine/threonine |
| | | | kinase |
| 55 | J04162 | FCGR3B | Fc fragment of IgG, low affinity |
| | | | IIIb, receptor for (CD16) |
| 56 | M60922 | FLOT2 | flotillin 2 |
| 57 | R72881 | GABBR1 | gamma-aminobutyric acid (GABA) |
| | | | B receptor, 1 |
| 58 | AF077740 | GCAT | glycine C-acetyltransferase (2- |
| | | | amino-3-ketobutyrate coenzyme A |
| | | | ligase) |
| 59 | M18185 | GIP | gastric inhibitory polypeptide |
| 60 | AA669536 | GJA5 | Gap junction protein, alpha 5, 40 kD |
| | | | (connexin 40) |
| 61 | U78027 | GLA | galactosidase, alpha |
| 62 | N26076 | GOV | glioblastoma overexpressed |
| 63 | D64154 | GP110 | cell membrane glycoprotein, |
| | | | 110000M(r) (surface antigen) |
| 64 | AF062006 | GPR49 | G protein-coupled receptor 49 |
| 65 | AA877534 | GPRC5C | G protein-coupled receptor, family |
| | | | C, group 5, member C |
| 66 | X68314 | GPX2 | glutathione peroxidase 2 |
| | | | (gastrointestinal) |
| 67 | AI346758 | GYG2 | glycogenin 2 |
| 68 | J04501 | GYS1 | glycogen synthase 1 (muscle) |
| 69 | U26174 | GZMK | granzyme K (serine protease, |
| | | | granzyme 3; tryptase II) |
| 70 | X57129 | H1F2 | H1 histone family, member 2 |
| 71 | AA904505 | H3FD | H3 histone family, member D |
| 72 | M16707 | H4F2 | H4 histone, family 2 |
| 73 | M58285 | HEM1 | hematopoietic protein 1 |
| 74 | AA903016 | HM74 | putative chemokine receptor; GTP- |
| | | | binding protein |
| 75 | D66904 | HRMT1L2 | HMT1 (hnRNP methyltransferase, |
| | | | S. cerevisiae)-like 2 |
| 76 | AW084318 | HSPB1 | heat shock 27 kD protein 1 |
| 77 | AA564686 | HSPC025 | HSPC025 |
| 78 | AA775500 | HsPOX2 | proline oxidase 2 |
| 79 | AI189477 | IDH2 | isocitrate dehydrogenase 2 |
| | | | (NADP+), mitochondrial |
| 80 | AA436509 | IER5 | Immediate early response 5 |
| 81 | X16302 | IGFBP2 | insulin-like growth factor binding |
| | | | protein 2 (36 kD) |
| 82 | AJ001563 | IGHG3 | immunoglobulin heavy constant |
| | | | gamma 3 (G3m marker) |
| 83 | M87790 | IGLλ | immunoglobulin lambda locus |
| 84 | AI189680 | IL1RAP | interleukin 1 receptor accessory |
| | | | protein |
| 85 | M20566 | IL6R | interleukin 6 receptor |
| 86 | J05272 | IMPDH1 | IMP (inosine monophosphate) |
| | | | dehydrogenase 1 |
| 87 | S78296 | INA | internexin neuronal intermediate |
| | | | filament protein, alpha |
| 88 | M15395 | ITGB2 | integrin, beta 2 (antigen CD18 |
| | | | (p95), lymphocyte function- |
| | | | associated antigen 1; macrophage |
| | | | antigen 1 (mac) beta subunit) |
| 89 | X16260 | ITIH1 | inter-alpha (globulin) inhibitor, H1 |
| | | | polypeptide |
| 90 | AA226073 | ITM2C | integral membrane protein 2C |
| 91 | AI205103 | ITPK1 | inositol 1,3,4-triphosphate 5/6 |
| | | | kinase |
| 92 | Z68228 | JUP | junction plakoglobin |
| 93 | AA707252 | KIAA0468 | Syndecan 3 (N-syndecan) |
| 94 | D52745 | KIAA0821 | lectomedin-2 |
| 95 | H06478 | KIF3C | kinesin family member 3C |
| 96 | U06698 | KIF5A | kinesin family member 5A |
| 97 | AA845512 | KLF4 | Kruppel-like factor 4 (gut) |
| 98 | X77744 | KR18 | KRAB zinc finger protein KR18 |
| 99 | X87342 | LLGL2 | lethal giant larvae (Drosophila) |
| | | | homolog 2 |
| 100 | BF971926 | LMNA | lamin A/C |
| 101 | AI298111 | LOC51116 | CGI-91 protein |
| 102 | AA714315 | LOC51181 | carbonyl reductase |
| 103 | D89078 | LTB4R | leukotriene b4 receptor (chemokine |
| | | | receptor-like 1) |
| 104 | U42376 | LY6E | lymphocyte antigen 6 complex, |
| | | | locus E |
| 105 | AC005546 | LYL1 | lymphoblastic leukemia derived |
| | | | sequence 1 |
| 106 | AA179832 | M6PR | mannose-6-phosphate receptor |
| | | | (cation dependent) |
| 107 | D87116 | MAP2K3 | mitogen-activated protein kinase |
| | | | kinase 3 |
| 108 | AA583183 | MAP4K3 | mitogen-activated protein kinase |
| | | | kinase kinase kinase 3 |
| 109 | AA744607 | MASL1 | MFH-amplified sequences with |
| | | | leucine-rich tandem repeats 1 |
| 110 | X74795 | MCM5 | minichromosome maintenance |
| | | | deficient (S. cerevisiae) 5 (cell |
| | | | division cycle 46) |
| 111 | U78313 | MDFI | MyoD family inhibitor |
| 112 | L10612 | MIF | macrophage migration inhibitory |
| | | | factor (glycosylation-inhibiting |
| | | | factor) |
| 113 | J05070 | MMP9 | matrix metalloproteinase 9 |
| | | | (gelatinase B, 92 kD gelatinase, |
| | | | 92 kD type IV collagenase) |
| 114 | H46518 | MRPS26 | Mitochondrial ribosomal protein |
| | | | S26 |
| 115 | AA101822 | MSDC1 | Mesoderm development candidate 1 |
| 116 | N70019 | MT1E | metallothionein 1E (functional) |
| 117 | AI094778 | MT2A | metallothionein 2A |
| 118 | J04031 | MTHFD1 | methylenetetrahydrofolate |
| | | | dehydrogenase (NADP+ dependent), |
| | | | methenyltetrahydrofolate |
| | | | cyclohydrolase, |
| | | | formyltetrahydrofolate synthetase |
| 119 | X13293 | MYBL2 | v-myb avian myeloblastosis viral |
| | | | oncogene homolog-like 2 |
| 120 | Y00664 | MYCN | V-myc avian myelocytomatosis viral |
| | | | related oncogene, neuroblastoma |
| | | | derived |
| 121 | AI188406 | NDUFA4 | NADH dehydrogenase (ubiquinone) |
| | | | 1 alpha subcomplex, 4 (9 kD, |
| | | | MLRQ) |
| 122 | AA989104 | NDUFB2 | NADH dehydrogenase (ubiquinone) |
| | | | 1 beta subcomplex, 2 (8 kD, AGGG) |
| 123 | X83957 | NEB | nebulin |
| 124 | H08616 | NESCA | nesca protein |
| 125 | AA977227 | NET-6 | tetraspan NET-6 protein |
| 126 | W46617 | NF2 | Neurofibromin 2 (bilateral acoustic |
| | | | neuroma) |
| 127 | AI300590 | NFE2L3 | nuclear factor (erythroid-derived 2)- |
| | | | like 3 |
| 128 | X77909 | NFKBIL1 | nuclear factor of kappa light |
| | | | polypeptide gene enhancer in B- |
| | | | cells inhibitor-like 1 |
| 129 | AJ001258 | NIPSNAP1 | NIPSNAP, C. elegans, homolog 1 |
| 130 | U23070 | NMA | putative transmembrane protein |
| 131 | X17620 | NME1 | non-metastatic cells 1, protein |
| | | | (NM23A) expressed in |
| 132 | L16785 | NME2 | non-metastatic cells 2, protein |
| | | | (NM23B) expressed in |
| 133 | AA242961 | NOD1 | caspase recruitment domain 4 |
| 134 | AI085648 | NOLA3 | nucleolar protein family A, member |
| | | | 3 (H/ACA small nucleolar RNPs) |
| 135 | U56079 | NPY5R | neuropeptide Y receptor Y5 |
| 136 | AA628440 | NR1I3 | nuclear receptor subfamily 1, group |
| | | | I, member 3 |
| 137 | R16767 | NRBP | nuclear receptor binding protein |
| 138 | AI049668 | OAZ1 | ornithine decarboxylase antizyme 1 |
| 139 | D10523 | OGDH | oxoglutarate dehydrogenase |
| | | | (lipoamide) |
| 140 | X17094 | PACE | paired basic amino acid cleaving |
| | | | enzyme (furin, membrane associated |
| | | | receptor protein) |
| 141 | AI146846 | PAR3 | three-PDZ containing protein similar |
| | | | to C. elegans PAR3 (partitioning |
| | | | defect) |
| 142 | AI248183 | PAX5 | Paired box gene 5 (B-cell lineage |
| | | | specific activator protein) |
| 143 | AI265770 | PDLIM1 | PDZ and LIM domain 1 (elfin) |
| 144 | X54936 | PGF | placental growth factor, vascular |
| | | | endothelial growth factor-related |
| | | | protein |
| 145 | AA532444 | PHLDA3 | pleckstrin homology-like domain, |
| | | | family A, member 3 |
| 146 | X80907 | PIK3R2 | phosphoinositide-3-kinase, |
| | | | regulatory subunit, polypeptide 2 |
| | | | (p85 beta) |
| 147 | M16750 | PIM1 | pim oncogene |
| 148 | U77735 | PIM2 | pim-2 oncogene |
| 149 | D00244 | PLAU | plasminogen activator, urokinase |
| 150 | X07743 | PLEK | pleckstrin |
| 151 | M80397 | POLD1 | polymerase (DNA directed), delta 1, |
| | | | catalytic subunit (125 kD) |
| 152 | S90469 | POR | P450 (cytochrome) oxidoreductase |
| 153 | AF045584 | POV1 | prostate cancer overexpressed gene 1 |
| 154 | S57501 | PPP1CA | protein phosphatase 1, catalytic |
| | | | subunit, alpha isoform |
| 155 | N44532 | PPP1R14C | Protein phosphatase 1, regulatory |
| | | | (inhibitor) subunit 14C |
| 156 | AI274279 | PRDM4 | PR domain containing 4 |
| 157 | AI309741 | PRG6 | p53-responsive gene 6 |
| 158 | AF027208 | PROML1 | prominin (mouse)-like 1 |
| 159 | M24398 | PTMS | parathymosin |
| 160 | U47025 | PYGB | phosphorylase, glycogen; brain |
| 161 | Y15233 | PYGL | phosphorylase, glycogen; liver (Hers |
| | | | disease, glycogen storage disease |
| | | | type VI) |
| 162 | AA346311 | RAI3 | retinoic acid induced 3 |
| 163 | M29893 | RALA | v-ral simian leukemia viral |
| | | | oncogene homolog A (ras related) |
| 164 | Y00291 | RARB | retinoic acid receptor, beta |
| 165 | Y12336 | RASGRP2 | RAS guanyl releasing protein 2 |
| | | | (calcium and DAG-regulated) |
| 166 | X64652 | RBMS1 | RNA binding motif, single stranded |
| | | | interacting protein 1 |
| 167 | AF040105 | RCL | putative c-Myc-responsive |
| 168 | AA807607 | RDGBB | retinal degeneration B beta |
| 169 | AA932768 | REPRIMO | candidate mediator of the p53- |
| | | | dependent G2 arrest |
| 170 | X12949 | RET | ret proto-oncogene (multiple |
| | | | endocrine neoplasia MEN2A, |
| | | | MEN2B and medullary thyroid |
| | | | carcinoma 1, Hirschsprung disease) |
| 171 | NM_139176 | PYPAF3 | PYRIN-containing Apaf-1-like |
| | | | protein 3 |
| 172 | AA921313 | RPL11 | ribosomal protein L11 |
| 173 | L11566 | RPL18 | ribosomal protein L18 |
| 174 | AA402920 | RPL18A | ribosomal protein L18a |
| 175 | AA962580 | RPL22 | ribosomal protein L22 |
| 176 | AI123363 | RPL23A | ribosomal protein L23a |
| 177 | AI341159 | RPL26 | ribosomal protein L26 |
| 178 | AA313541 | RPL37 | ribosomal protein L37 |
| 179 | R50505 | RPLP1 | ribosomal protein, large, P1 |
| 180 | AI131289 | RPLP2 | ribosomal protein, large P2 |
| 181 | M81757 | RPS19 | ribosomal protein S19 |
| 182 | L04483 | RPS21 | ribosomal protein S21 |
| 183 | N27409 | RPS23 | ribosomal protein S23 |
| 184 | U14970 | RPS5 | ribosomal protein S5 |
| 185 | X99920 | S100A13 | S100 calcium-binding protein A13 |
| 186 | AI261620 | SAAS | granin-like neuroendocrine peptide |
| | | | precursor |
| 187 | U72355 | SAFB | scaffold attachment factor B |
| 188 | X98834 | SALL2 | sal (Drosophila)-like 2 |
| 189 | T30682 | SCO2 | SCO cytochrome oxidase deficient |
| | | | homolog 2 (yeast) |
| 190 | AB000887 | SCYA19 | small inducible cytokine subfamily |
| | | | A (Cys—Cys), member 19 |
| 191 | AA534943 | SCYB14 | small inducible cytokine subfamily |
| | | | B (Cys-X-Cys), member 14 (BRAK) |
| 192 | AI080351 | SEC63L | SEC63, endoplasmic reticulum |
| | | | translocon component (S. cerevisiae) |
| | | | like |
| 193 | K01396 | SERPINA1 | serine (or cysteine) proteinase |
| | | | inhibitor, clade A (alpha |
| | | | antiproteinase, antitrypsin), member 1 |
| 194 | AI050752 | SGCB | Sarcoglycan, beta (43 kD dystrophin- |
| | | | associated glycoprotein) |
| 195 | AA421248 | SH3BGRL3 | SH3 domain binding glutamic acid- |
| | | | rich protein like 3 |
| 196 | L11932 | SHMT1 | serine hydroxymethyltransferase 1 |
| 197 | T29731 | SHMT2 | serine hydroxymethyltransferase 2 |
| | | | (mitochondrial) |
| 198 | U44403 | SLA | Src-like-adapter |
| 199 | J03592 | SLC25A6 | solute carrier family 25 |
| | | | (mitochondrial carrier; adenine |
| | | | nucleotide translocator), member 6 |
| 200 | AW511361 | SLC29A1 | solute carrier family 29 (nucleoside |
| | | | transporters), member 1 |
| 201 | D84454 | SLC35A2 | solute carrier family 35 (UDP- |
| | | | galactose transporter), member 2 |
| 202 | M65105 | SLC6A2 | solute carrier family 6 |
| | | | (neurotransmitter transporter, |
| | | | noradrenalin), member 2 |
| 203 | AW504047 | SMARCA4 | SWI/SNF related, matrix associated, |
| | | | actin dependent regulator of |
| | | | chromatin, subfamily a, member 4 |
| 204 | AI143147 | SNRPF | small nuclear ribonucleoprotein |
| | | | polypeptide F |
| 205 | X70683 | SOX4 | SRY (sex determining region Y)- |
| | | | box 4 |
| 206 | U49240 | SPK | symplekin; Huntingtin interacting |
| | | | protein I |
| 207 | J03161 | SRF | serum response factor (c-fos serum |
| | | | response element-binding |
| | | | transcription factor) |
| 208 | AA683542 | STAU2 | staufen (Drosophila, RNA-binding |
| | | | protein) homolog 2 |
| 209 | AI151087 | T1A-2 | lung type-I cell membrane- |
| | | | associated glycoprotein |
| 210 | AA235074 | TCF19 | transcription factor 19 (SC1) |
| 211 | X82240 | TCL1A | T-cell leukemia/lymphoma 1A |
| 212 | AA399645 | TCOF1 | Treacher Collins-Franceschetti |
| | | | syndrome 1 |
| 213 | U85658 | TFAP2C | transcription factor AP-2 gamma |
| | | | (activating enhancer-binding protein |
| | | | 2 gamma) |
| 214 | AI049960 | TGIF2 | TGFB-induced factor 2 (TALE |
| | | | family homeobox) |
| 215 | AA293042 | THY1 | Thy cell surface antigen |
| 216 | AJ005895 | TIM17B | translocase of inner mitochondrial |
| | | | membrane 17 (yeast) homolog B |
| 217 | AA536113 | TMEPAI | transmembrane, prostate androgen |
| | | | induced RNA |
| 218 | AI261341 | TMP21 | transmembrane trafficking protein |
| 219 | M64247 | TNNI3 | troponin I, cardiac |
| 220 | M19309 | TNNT1 | troponin T1, skeletal, slow |
| 221 | M19713 | TPM1 | tropomyosin 1 (alpha) |
| 222 | AA890188 | TUBG2 | tubulin, gamma 2 |
| 223 | AA481924 | TYROBP | TYRO protein tyrosine kinase |
| | | | binding protein |
| 224 | U73379 | UBCH10 | ubiquitin carrier protein E2-C |
| 225 | AA465240 | VAV2 | vav 2 oncogene |
| 226 | Z71621 | WNT2B | wingless-type MMTV integration |
| | | | site family, member 2B |
| 227 | AA644644 | YWHAH | tyrosine 3- |
| | | | monooxygenase/tryptophan 5- |
| | | | monooxygenase activation protein, |
| | | | eta polypeptide |
| 228 | AA555115 | LOC51260 | hypothetical protein |
| 229 | AA056472 | LOC57228 | hypothetical protein from clone 643 |
| 230 | R37098 | DKFZp547M236 | hypothetical protein |
| | | | DKFZp547M236 |
| 231 | AA776240 | DKFZP586J0917 | DKFZP586J0917 protein |
| 232 | AA609417 | DKFZp762M136 | hypothetical protein |
| | | | DKFZp762M136 |
| 233 | N80485 | FLJ10199 | hypothetical protein FLJ10199 |
| 234 | W18181 | FLJ10430 | hypothetical protein FLJ10430 |
| 235 | U69190 | FLJ10432 | hypothetical protein |
| 236 | AA287875 | FLJ10549 | hypothetical protein FLJ10549 |
| 237 | AI206219 | FLJ10634 | hypothetical protein FLJ10634 |
| 238 | AA368409 | FLJ10688 | hypothetical protein FLJ10688 |
| 239 | AI014673 | FLJ10709 | hypothetical protein FLJ10709 |
| 240 | AA219141 | FLJ10713 | hypothetical protein FLJ10713 |
| 241 | AA477929 | FLJ10767 | hypothetical protein FLJ10767 |
| 242 | AK026707 | FLJ11328 | hypothetical protein FLJ11328 |
| 243 | AA306716 | FLJ11937 | hypothetical protein FLJ11937 |
| 244 | AI017753 | FLJ20069 | hypothetical protein FLJ20069 |
| 245 | AA843844 | FLJ20171 | hypothetical protein FLJ20171 |
| 246 | AI360274 | FLJ20494 | similar to mouse neuronal protein |
| | | | 15.6 |
| 247 | AI276023 | FLJ20539 | hypothetical protein FLJ20539 |
| 248 | AA058761 | FLJ20550 | hypothetical protein FLJ20550 |
| 249 | Z24980 | FLJ22195 | hypothetical protein FLJ22195 |
| 250 | AA813912 | KIAA0130 | KIAA0130 gene product |
| 251 | AA394063 | KIAA0144 | KIAA0144 gene product |
| 252 | AI090862 | KIAA0147 | human homolog of Drosophila |
| | | | Scribble |
| 253 | AB007925 | KIAA0456 | KIAA0456 protein |
| 254 | AB014544 | KIAA0644 | KIAA0644 gene product |
| 255 | AB014590 | KIAA0690 | KIAA0690 protein |
| 256 | AA954348 | KIAA0870 | KIAA0870 protein |
| 257 | AA737525 | KIAA1031 | KIAA1031 protein |
| 258 | AA443202 | KIAA1053 | KIAA1053 protein |
| 259 | W90578 | KIAA1198 | KIAA1198 protein |
| 260 | AA191449 | KIAA1254 | KIAA1254 protein |
| 261 | AI076459 | KIAA1272 | Homo sapiens cDNA FLJ12819 fis, |
| | | | clone NT2RP2002727, weakly |
| | | | similar to Rattus norvegicus tulip 2 |
| | | | mRNA |
| 262 | AA579859 | KIAA1273 | KIAA1273 protein |
| 263 | AA731891 | KIAA1517 | KIAA1517 protein |
| 264 | AI093595 | LOC55895 | 22 kDa peroxisomal membrane |
| | | | protein-like |
| 265 | AA149846 | | Homo sapiens mRNA; cDNA |
| | | | DKFZp762B195 (from clone |
| | | | DKFZp762B195) |
| 266 | AA741366 | | Homo sapiens mRNA; cDNA |
| | | | DKFZp761K2312 (from clone |
| | | | DKFZp761K2312) |
| 267 | AA400449 | DKFZp434K0621 | Homo sapiens mRNA; cDNA |
| | | | DKFZp434K0621 (from clone |
| | | | DKFZp434K0621); partial cds |
| 268 | AI168147 | | Homo sapiens HSPC289 mRNA, |
| | | | partial cds |
| 269 | L02326 | | Homo sapiens clone Hu lambda7 |
| | | | lambda-like protein (IGLL2) gene, |
| | | | partial cds |
| 270 | F09520 | EST | Homo sapiens clone 24841 mRNA |
| | | | sequence |
| 271 | AA975205 | | Homo sapiens clone 23570 mRNA |
| | | | sequence |
| 272 | AI348289 | | Homo sapiens cDNA: FLJ23227 fis, |
| | | | clone CAE00645, highly similar to |
| | | | AF052138 Homo sapiens clone |
| | | | 23718 mRNA sequence |
| 273 | AA669034 | | Homo sapiens cDNA: FLJ23125 fis, |
| | | | clone LNG08217 |
| 274 | W76303 | | Homo sapiens cDNA: FLJ22662 fis, |
| | | | clone HSI08080 |
| 275 | T04932 | | Homo sapiens cDNA: FLJ21545 fis, |
| | | | clone COL06195 |
| 276 | AA147751 | | Homo sapiens cDNA FLJ14146 fis, |
| | | | clone MAMMA1002947 |
| 277 | N91027 | | Homo sapiens cDNA FLJ13549 fis, |
| | | | clone PLACE1007097 |
| 278 | AA188494 | FLJ113352 | Homo sapiens cDNA FLJ13352 fis, |
| | | | clone OVARC1002165, weakly |
| | | | similar to 3-OXO-5-ALPHA- |
| | | | STEROID 4-DEHYDROGENASE |
| | | | 2 (EC 1.3.99.5) |
| 279 | AA903456 | | Homo sapiens cDNA FLJ13325 fis, |
| | | | clone OVARC1001762, weakly |
| | | | similar to N-TERMINAL |
| | | | ACETYLTRANSFERASE 1 (EC |
| | | | 2.3.1.88) |
| 280 | AA628522 | | Homo sapiens cDNA FLJ12758 fis, |
| | | | clone NT2RP2001328 |
| 281 | AA626414 | | Homo sapiens cDNA FLJ12436 fis, |
| | | | clone NT2RM1000062 |
| 282 | AA610175 | FLJ12195 | Homo sapiens cDNA FLJ12195 fis, |
| | | | clone MAMMA1000865 |
| 283 | AW083127 | | Homo sapiens cDNA FLJ11856 fis, |
| | | | clone HEMBA1006789 |
| 284 | F18016 | | Homo sapiens cDNA FLJ11018 fis, |
| | | | clone PLACE1003602, highly |
| | | | similar to Homo sapiens mRNA |
| | | | expressed in plcenta |
| 285 | AA442071 | EST | Homo sapiens cDNA FLJ10247 fis, |
| | | | clone HEMBB1000705 |
| 286 | AA036947 | | Homo sapiens cDNA FLJ10229 fis, |
| | | | clone HEMBB1000136 |
| 287 | AA234475 | NCOA6IP | PRIP-interacting protein with |
| | | | methyltransferase domain |
| 288 | AI041186 | | HSPC182 protein |
| 289 | K01505 | | DC classII histocompatibility |
| | | | antigen alpha-chain |
| 290 | Z38677 | | Claudin 10 |
| 291 | AA236315 | | Chromosome 1 open reading frame |
| | | | 27 |
| 292 | AA411333 | | ESTs, Weakly similar to zinc |
| | | | finger-like [H. sapiens] |
| 293 | AA150200 | | ESTs, Weakly similar to tuftelin |
| | | | [M. musculus] |
| 294 | AI341906 | | ESTs, Weakly similar to ORF |
| | | | YNL310c [S. cerevisiae] |
| 295 | AI349804 | EST | ESTs, Weakly similar to |
| | | | IQGA_HUMAN RAS GTPASE- |
| | | | ACTIVATING-LIKE PROTEIN |
| | | | IQGAP1 [H. sapiens] |
| 296 | W94363 | | ESTs, Weakly similar to |
| | | | ALU4_HUMAN ALU |
| | | | SUBFAMILY SB2 SEQUENCE |
| | | | CONTAMINATION WARNING |
| | | | ENTRY [H. sapiens] |
| 297 | AA053248 | | ESTs, Highly similar to |
| | | | RS10_HUMAN 40S RIBOSOMAL |
| | | | PROTEIN S10 [H. sapiens] |
| 298 | AA514648 | | ESTs, Highly similar to |
| | | | LMA1_HUMAN LAMININ |
| | | | ALPHA CHAIN PRECURSOR |
| | | | [H. sapiens] |
| 299 | T03298 | | ESTs, Highly similar to |
| | | | LDHH_HUMAN L-LACTATE |
| | | | DEHYDROGENASE H CHAIN |
| | | | [H. sapiens] |
| 300 | T55019 | | ESTs, fetal spleen |
| 301 | AI088718 | | ESTs |
| 302 | AA024920 | | ESTs |
| 303 | R77448 | PLXNA2 | ESTs |
| 304 | W31174 | | ESTs |
| 305 | AA463626 | | ESTs |
| 306 | AI344249 | | ESTs |
| 307 | R61891 | | ESTs |
| 308 | AA479350 | | ESTs |
| 309 | AA327207 | | ESTs |
| 310 | AA528140 | | ESTs |
| 311 | AA826148 | EST | ESTs |
| 312 | AA913950 | | ESTs |
| 313 | AI243620 | | ESTs |
| 314 | AI039201 | | ESTs |
| 315 | AA936889 | | ESTs |
| 316 | AA687757 | | ESTs |
| 317 | AI366259 | | ESTs |
| 318 | AA317670 | | ESTs |
| 319 | AI141923 | | ESTs |
| 320 | AA778238 | EST | ESTs |
| 321 | T72555 | | ESTs |
| 322 | AA602585 | | ESTs |
| 323 | AA527570 | | ESTs |
| 324 | C75253 | | ESTs |
| 325 | AA351680 | | ESTs |
| 326 | N75945 | | ESTs |
| 327 | AA528243 | | ESTs |
| 328 | AA688195 | | ESTs |
| 329 | AA063157 | | ESTs |
| 330 | AA419568 | | ESTs |
| 331 | D85376 | | ESTs |
| 332 | AA521342 | | ESTs |
| 333 | AI365844 | | ESTs |
| 334 | T55926 | | ESTs |
| 335 | R94687 | | ESTs |
| 336 | T61564 | | ESTs |
| 337 | AI305234 | LOC152217 | ESTs |
| 338 | AA233870 | | ESTs |
| 339 | T16470 | | ESTs |
| 340 | T16802 | | ESTs |
| 341 | AA830668 | EST | EST |
| 342 | AA489212 | | EST |
| 343 | AA758394 | | EST |
| 344 | AA609658 | | EST |
| 345 | AA683373 | | EST |
| 346 | N34387 | | EST |

TABLE 4
593 genes commomly 0.2 fold down-regulated or less in testicular seminomas.

| TS | | | |
|---|---|---|---|
| Assignment | Accession No. | Symbol | Gene name |
| 347 | U57961 | 13CDNA73 | putative gene product |
| 348 | M35296 | ABL2 | v-abl Abelson murine leukemia |
| | | | viral oncogene homolog 2 (arg, |
| | | | Abelson-related gene) |
| 349 | AA406601 | ABLIM | actin binding LIM protein 1 |
| 350 | AA815365 | ACT | activator of CREM in testis |
| 351 | AI357650 | AD026 | AD026 protein |
| 352 | AF029900 | ADAM21 | a disintegrin and |
| | | | metalloproteinase domain 21 |
| 353 | X74210 | ADCY2 | adenylate cyclase 2 (brain) |
| 354 | X03350 | ADH2 | alcohol dehydrogenase 2 (class I), |
| | | | beta polypeptide |
| 355 | L22214 | ADORA1 | adenosine A1 receptor |
| 356 | X66503 | ADSS | adenylosuccinate synthase |
| 357 | AA766028 | AF15Q14 | AF15q14 protein |
| 358 | AA434178 | AGPAT1 | 1-acylglycerol-3-phosphate O- |
| | | | acyltransferase 1 |
| | | | (lysophosphatidic acid |
| | | | acyltransferase, alpha) |
| 359 | AF038564 | AIP4 | atrophin interacting protein 4 |
| 360 | AI028271 | AKAP3 | A kinase (PRKA) anchor protein 3 |
| 361 | AA398240 | AKAP4 | A kinase (PRKA) anchor protein 4 |
| 362 | U05861 | AKR1C1 | aldo-keto reductase family 1, |
| | | | member C1 (dihydrodiol |
| | | | dehydrogenase 1; 20-alpha (3- |
| | | | alpha)-hydroxysteroid |
| | | | dehydrogenase) |
| 363 | D17793 | AKR1C3 | aldo-keto reductase family 1, |
| | | | member C3 (3-alpha |
| | | | hydroxysteroid dehydrogenase, |
| | | | type II) |
| 364 | K03000 | ALDH1 | aldehyde dehydrogenase 1, |
| | | | soluble |
| 365 | M18786 | AMY1A | amylase, alpha 1A; salivary |
| 366 | M19383 | ANXA4 | annexin A4 |
| 367 | Y12226 | AP1G1 | adaptor-related protein complex |
| | | | 1, gamma 1 subunit |
| 368 | AI278652 | AP1S2 | adaptor-related protein complex |
| | | | 1, sigma 2 subunit |
| 369 | AA421206 | APG | heat shock protein (hsp110 |
| | | | family) |
| 370 | AI168526 | ARHGAP5 | Rho GTPase activating protein 5 |
| 371 | AI025137 | ARHGEF3 | Rho guanine nucleotide exchange |
| | | | factor (GEF) 3 |
| 372 | AB002305 | ARNT2 | aryl-hydrocarbon receptor nuclear |
| | | | translocator 2 |
| 373 | U47054 | ART3 | ADP-ribosyltransferase 3 |
| 374 | AA928117 | ATP8A2 | ATPase, aminophospholipid |
| | | | transporter-like, Class I, type 8A, |
| | | | member 2 |
| 375 | H80325 | BAZ1A | bromodomain adjacent to zinc |
| | | | finger domain, 1A |
| 376 | M55575 | BCKDHB | branched chain keto acid |
| | | | dehydrogenase E1, beta |
| | | | polypeptide (maple syrup urine |
| | | | disease) |
| 377 | D87461 | BCL2L2 | BCL2-like 2 |
| 378 | AA620708 | BCLG | Apoptosis regulator BCL-G |
| 379 | U70824 | BLu | BLu protein |
| 380 | AA916688 | BRF1 | butyrate response factor 1 (EGF- |
| | | | response factor 1) |
| 381 | U03274 | BTD | biotinidase |
| 382 | D31716 | BTEB1 | basic transcription element |
| | | | binding protein 1 |
| 383 | W45244 | C3 | complement component 3 |
| 384 | U36448 | CADPS | Ca2+-dependent activator protein |
| | | | for secretion |
| 385 | X56667 | CALB2 | calbindin 2, (29 kD, calretinin) |
| 386 | AA600048 | CALD1 | caldesmon 1 |
| 387 | R39610 | CAPN2 | calpain 2, (m/II) large subunit |
| 388 | AI085802 | CAV2 | Caveolin 2 |
| 389 | M58583 | CBLN1 | cerebellin 1 precursor |
| 390 | D78333 | CCT6B | chaperonin containing TCP1, |
| | | | subunit 6B (zeta 2) |
| 391 | AA917718 | CDC10 | CDC10 (cell division cycle 10, S. cerevisiae, |
| | | | homolog) |
| 392 | L27711 | CDKN3 | cyclin-dependent kinase inhibitor |
| | | | 3 (CDK2-associated dual |
| | | | specificity phosphatase) |
| 393 | AI140736 | CDV | CDV protein |
| 394 | AF083322 | CEP1 | centrosomal protein 1 |
| 395 | AI142230 | CETN3 | centrin, EF-hand protein, 3 |
| | | | (CDC31 yeast homolog) |
| 396 | J03483 | CHGA | chromogranin A (parathyroid |
| | | | secretory protein 1) |
| 397 | D10704 | CHK | choline kinase |
| 398 | AA400791 | CHST3 | Carbohydrate (chondroitin |
| | | | 6/keratan) sulfotransferase 3 |
| 399 | U65092 | CITED1 | Cbp/p300-interacting |
| | | | transactivator, with Glu/Asp-rich |
| | | | carboxy-terminal domain, 1 |
| 400 | AI333035 | CKAP2 | cytoskeleton associated protein 2 |
| 401 | AI078139 | CKN1 | Cockayne syndrome 1 (classical) |
| 402 | D86322 | CLGN | calmegin |
| 403 | M64722 | CLU | clusterin (complement lysis inhibitor, |
| | | | SP-40,40, sulfated glycoprotein |
| | | | 2, testosterone-repressed |
| | | | prostate message 2, apolipoprotein |
| | | | J) |
| 404 | D17408 | CNN1 | calponin 1, basic, smooth muscle |
| 405 | L25286 | COL15A1 | collagen, type XV, alpha 1 |
| 406 | T93566 | CPE | carboxypeptidase E |
| 407 | F21182 | CRAT | carnitine acetyltransferase |
| 408 | AI334396 | CRSP9 | cofactor required for Sp1 |
| | | | transcriptional activation, subunit |
| | | | 9 (33 kD) |
| 409 | M55268 | CSNK2A2 | casein kinase 2, alpha prime |
| | | | polypeptide |
| 410 | X16312 | CSNK2B | casein kinase 2, beta polypeptide |
| 411 | U16306 | CSPG2 | chondroitin sulfate proteoglycan 2 |
| | | | (versican) |
| 412 | M33146 | CSRP1 | cysteine and glycine-rich protein 1 |
| 413 | AA780301 | CTSF | cathepsin F |
| 414 | AB001928 | CTSL2 | cathepsin L2 |
| 415 | AA417733 | CUL1 | cullin 1 |
| 416 | Z22780 | CYLC1 | cylicin, basic protein of sperm |
| | | | head cytoskeleton 1 |
| 417 | M14564 | CYP17 | cytochrome P450, subfamily |
| | | | XVII (steroid 17-alpha- |
| | | | hydroxylase), adrenal hyperplasia |
| 418 | U62015 | CYR61 | cysteine-rich, angiogenic inducer, |
| | | | 61 |
| 419 | AA608804 | D6S51E | HLA-B associated transcript-2 |
| 420 | AA640753 | DDAH1 | dimethylarginine |
| | | | dimethylaminohydrolase 1 |
| 421 | X62535 | DGKA | diacylglycerol kinase, alpha |
| | | | (80 kD) |
| 422 | AI209130 | DJ402G11.8 | novel protein similar to mouse |
| | | | MOV10 |
| 423 | AA432207 | DMRT1 | doublesex and mab-3 related |
| | | | transcription factor 1 |
| 424 | AJ000522 | DNAH17 | dynein, axonemal, heavy |
| | | | polypeptide 17 |
| 425 | U53445 | DOC1 | downregulated in ovarian cancer 1 |
| 426 | AA488466 | DRG1 | developmentally regulated GTP- |
| | | | binding protein 1 |
| 427 | X68277 | DUSP1 | dual specificity phosphatase 1 |
| 428 | AA313118 | DUSP10 | dual specificity phosphatase 10 |
| 429 | U89278 | EDR2 | early development regulator 2 |
| | | | (homolog of polyhomeotic 2) |
| 430 | M62829 | EGR1 | early growth response 1 |
| 431 | AA398573 | EIF5A2 | eukaryotic translation initiation |
| | | | factor 5A2 |
| 432 | AI097529 | EPAS1 | endothelial PAS domain protein 1 |
| 433 | U62740 | EXT2 | exostoses (multiple) 2 |
| 434 | M14354 | F13A1 | coagulation factor XIII, A1 |
| | | | polypeptide |
| 435 | D10040 | FACL2 | fatty-acid-Coenzyme A ligase, |
| | | | long-chain 2 |
| 436 | L13923 | FBN1 | fibrillin 1 (Marfan syndrome) |
| 437 | AI194045 | FE65L2 | FE65-LIKE 2 |
| 438 | AI351061 | FEM1B | FEM (C. elegans) homolog b |
| 439 | D14446 | FGL1 | fibrinogen-like 1 |
| 440 | U60115 | FHL1 | four and a half LIM domains 1 |
| 441 | AA678103 | FKBP5 | FK506-binding protein 5 |
| 442 | L37033 | FKBP8 | FK506-binding protein 8 (38 kD) |
| 443 | AA876478 | FLJ10578 | Sec61 alpha form 2 |
| 444 | AI141417 | FLJ10873 | UDP-glucose:glycoprotein |
| | | | glucosyltransferase 2 |
| 445 | AA813008 | FOP | FGFR1 oncogene partner |
| 446 | X74142 | FOXG1B | forkhead box G1B |
| 447 | AI025916 | FSP-2 | fibrousheathin II |
| 448 | X03674 | G6PD | glucose-6-phosphate |
| | | | dehydrogenase |
| 449 | N34138 | GABARAP | GABA(A) receptor-associated |
| | | | protein |
| 450 | U13044 | GABPA | GA-binding protein transcription |
| | | | factor, alpha subunit (60 kD) |
| 451 | S68805 | GATM | glycine amidinotransferase (L- |
| | | | arginine:glycine |
| | | | amidinotransferase) |
| 452 | AA583339 | GCNT3 | glucosaminyl (N-acetyl) |
| | | | transferase 3, mucin type |
| 453 | AI014575 | GCP60 | golgi resident protein GCP60 |
| 454 | AA578014 | GGA1 | ADP-ribosylation factor binding |
| | | | protein GGA1 |
| 455 | AA523541 | GILZ | glucocorticoid-induced leucine |
| | | | zipper |
| 456 | AA293636 | GJA1 | gap junction protein, alpha 1, |
| | | | 43 kD (connexin 43) |
| 457 | AA608780 | GKP2 | Glycerol kinase pseudogene 2 |
| 458 | AA887118 | GLRX2 | Glutaredoxin 2 |
| 459 | AA446421 | GMPS | guanine monphosphate synthetase |
| 460 | AF055013 | GNAI1 | guanine nucleotide binding |
| | | | protein (G protein), alpha |
| | | | inhibiting activity polypeptide 1 |
| 461 | AA401492 | GNAS1 | guanine nucleotide binding |
| | | | protein (G protein), alpha |
| | | | stimulating activity polypeptide 1 |
| 462 | AF007548 | GOSR2 | golgi SNAP receptor complex |
| | | | member 2 |
| 463 | AA031372 | GPC4 | glypican 4 |
| 464 | AI126171 | GPP130 | type II Golgi membrane protein |
| 465 | L42324 | GPR18 | G protein-coupled receptor 18 |
| 466 | X71973 | GPX4 | glutathione peroxidase 4 |
| | | | (phospholipid hydroperoxidase) |
| 467 | L76687 | GRB14 | growth factor receptor-bound |
| | | | protein 14 |
| 468 | AI015487 | GRTH | gonadotropin-regulated testicular |
| | | | RNA helicase |
| 469 | D87119 | GS3955 | GS3955 protein |
| 470 | AA993251 | GSTA2 | glutathione S-transferase A2 |
| 471 | L13275 | GSTA3 | glutathione S-transferase A3 |
| 472 | L02321 | GSTM5 | glutathione S-transferase M5 |
| 473 | U14193 | GTF2A2 | general transcription factor IIA, 2 |
| | | | (12 kD subunit) |
| 474 | AI126491 | HBACH | Cytosolic acyl coenzyme A |
| | | | thioester hydrolase |
| 475 | AF019214 | HBP1 | HMG-box containing protein 1 |
| 476 | W95267 | HIBADH | 3-hydroxyisobutyrate |
| | | | dehydrogenase |
| 477 | U40992 | HLJ1 | DnaJ-like heat shock protein 40 |
| 478 | M11058 | HMGCR | 3-hydroxy-3-methylglutaryl- |
| | | | Coenzyme A reductase |
| 479 | X83618 | HMGCS2 | 3-hydroxy-3-methylglutaryl- |
| | | | Coenzyme A synthase 2 |
| | | | (mitochondrial) |
| 480 | AI215478 | HMMR | hyaluronan-mediated motility |
| | | | receptor (RHAMM) |
| 481 | Y09980 | HOXD3 | homeo box D3 |
| 482 | AF070616 | HPCAL1 | hippocalcin-like 1 |
| 483 | Y12711 | HPR6.6 | progesterone binding protein |
| 484 | AA825654 | HRB | HIV Rev binding protein |
| 485 | AI027700 | HS1-2 | putative transmembrane protein |
| 486 | M65217 | HSF2 | heat shock transcription factor 2 |
| 487 | AI205684 | HSPA2 | heat shock 70 kD protein 2 |
| 488 | AA971601 | HSSOX6 | SRY (sex determining region Y)- |
| | | | box 6 |
| 489 | AA493561 | IGSF4 | immunoglobulin superfamily, |
| | | | member 4 |
| 490 | AA916823 | IL1A | interleukin 1, alpha |
| 491 | M27492 | IL1R1 | interleukin 1 receptor, type I |
| 492 | D61009 | ING1L | inhibitor of growth family, |
| | | | member 1-like |
| 493 | L08488 | INPP1 | inositol polyphosphate- |
| | | | phosphatase |
| 494 | AI192189 | INPP5A | inositol polyphosphate-5- |
| | | | phosphatase, 40 kD |
| 495 | W76477 | JUN | v-jun avian sarcoma virus 17 |
| | | | oncogene homolog |
| 496 | AA933702 | KCNK4 | potassium inwardly-rectifying |
| | | | channel, subfamily K, member 4 |
| 497 | U25138 | KCNMB1 | potassium large conductance |
| | | | calcium-activated channel, |
| | | | subfamily M, beta member 1 |
| 498 | AF064093 | KEO4 | similar to Caenorhabditis elegans |
| | | | protein C42C1.9 |
| 499 | D14661 | KIAA0105 | Wilms' tumour 1-associating |
| | | | protein |
| 500 | AB014531 | KIAA0631 | very long-chain acyl-CoA |
| | | | synthetase; lipidosin |
| 501 | H98203 | KIAA0987 | differentially expressed in |
| | | | adenocarcinoma of the lung |
| 502 | AA037452 | KIAA0992 | palladin |
| 503 | Y08319 | KIF2 | kinesin heavy chain member 2 |
| 504 | AL044356 | KPNB3 | karyopherin (importin) beta 3 |
| 505 | M59832 | LAMA2 | laminin, alpha 2 (merosin, |
| | | | congenital muscular dystrophy) |
| 506 | AF064492 | LDB2 | LIM domain binding 2 |
| 507 | L13210 | LGALS3BP | lectin, galactoside-binding, |
| | | | soluble, 3 binding protein |
| | | | (galectin 6 binding protein) |
| 508 | AA252389 | LHFP | lipoma HMGIC fusion partner |
| 509 | AA191662 | LOC51617 | HMP19 protein |
| 510 | AI160184 | LOC51673 | brain specific protein |
| 511 | AA569922 | LOC51706 | cytochrome b5 reductase 1 |
| | | | (B5R.1) |
| 512 | AA527435 | LOC63928 | hepatocellular carcinoma antigen |
| | | | gene 520 |
| 513 | AA173168 | LRRFIP2 | leucine rich repeat (in FLII) |
| | | | interacting protein 2 |
| 514 | M83202 | LTF | lactotransferrin |
| 515 | AA459595 | LZK1 | C3HC4-type zinc finger protein |
| 516 | U44378 | MADH4 | MAD (mothers against |
| | | | decapentaplegic, Drosophila) |
| | | | homolog 4 |
| 517 | X74837 | MAN1A1 | mannosidase, alpha, class 1A, |
| | | | member 1 |
| 518 | M69226 | MAOA | monoamine oxidase A |
| 519 | AA157731 | MAP1ALC3 | Microtubule-associated proteins |
| | | | 1A and 1B, light chain 3 |
| 520 | U07620 | MAPK10 | mitogen-activated protein kinase |
| | | | 10 |
| 521 | D10511 | MAT | mitochondrial acetoacetyl-CoA |
| | | | thiolase |
| 522 | X68836 | MAT2A | methionine adenosyltransferase |
| | | | II, alpha |
| 523 | AA228022 | MCAM | melanoma adhesion molecule |
| 524 | X12556 | MCF2 | MCF2 cell line derived |
| | | | transforming sequence |
| 525 | AI215620 | MCSP | mitochondrial capsule |
| | | | selenoprotein |
| 526 | AA815051 | MDG1 | microvascular endothelial |
| | | | differentiation gene 1 |
| 527 | L38486 | MFAP4 | microfibrillar-associated protein 4 |
| 528 | AA135566 | MGEA6 | meningioma expressed antigen 6 |
| | | | (coiled-coil proline-rich) |
| 529 | X53331 | MGP | matrix Gla protein |
| 530 | U77604 | MGST2 | microsomal glutathione S- |
| | | | transferase 2 |
| 531 | M16279 | MIC2 | antigen identified by monoclonal |
| | | | antibodies 12E7, F21 and O13 |
| 532 | U38320 | MMP19 | matrix metalloproteinase 19 |
| 533 | M93405 | MMSDH | methylmalonate-semialdehyde |
| | | | dehydrogenase |
| 534 | AI140756 | MP1 | metalloprotease 1 (pitrilysin |
| | | | family) |
| 535 | AA868815 | MSL3L1 | male-specific lethal-3 |
| | | | (Drosophila)-like 1 |
| 536 | X59657 | MTP | microsomal triglyceride transfer |
| | | | protein (large polypeptide, 88 kD) |
| 537 | J05581 | MUC1 | mucin 1, transmembrane |
| 538 | AA401638 | MUL | Mulibrey nanism |
| 539 | AA319638 | MYH9 | Myosin, heavy polypeptide 9, |
| | | | non-muscle |
| 540 | X85337 | MYLK | myosin, light polypeptide kinase |
| 541 | D87930 | MYPT1 | myosin phosphatase, target |
| | | | subunit 1 |
| 542 | J02854 | MYRL2 | myosin regulatory light chain 2, |
| | | | smooth muscle isoform |
| 543 | D50370 | NAP1L3 | nucleosome assembly protein 1- |
| | | | like 3 |
| 544 | AA906200 | NAP4 | Nck, Ash and phospholipase C |
| | | | binding protein |
| 545 | AA855085 | NCOA4 | nuclear receptor coactivator 4 |
| 546 | U22897 | NDP52 | nuclear domain 10 protein |
| 547 | AI088622 | NDUFS2 | NADH dehydrogenase |
| | | | (ubiquinone) Fe—S protein 2 |
| | | | (49 kD) (NADH-coenzyme Q |
| | | | reductase) |
| 548 | Y00067 | NEF3 | neurofilament 3 (150 kD medium) |
| 549 | M58603 | NFKB1 | nuclear factor of kappa light |
| | | | polypeptide gene enhancer in B- |
| | | | cells 1 (p105) |
| 550 | U83843 | NIP7-1 | HIV-1 Nef interacting protein |
| 551 | AA707108 | NKX3A | NK homeobox (Drosophila), |
| | | | family 3, A |
| 552 | AA340728 | NR2F2 | nuclear receptor subfamily 2, |
| | | | group F, member 2 |
| 553 | AA215284 | NSF | N-ethylmaleimide-sensitive factor |
| 554 | X55740 | NT5 | 5′ nucleotidase (CD73) |
| 555 | X76732 | NUCB2 | nucleobindin 2 |
| 556 | AJ007558 | NUP155 | nucleoporin 155 kD |
| 557 | AA902823 | NYD-SP12 | NYD-SP12 protein |
| 558 | AA699559 | NYD-SP15 | Protein kinase NYD-SP15 |
| 559 | AI208877 | NYD-SP21 | Testes development-related NYD- |
| | | | SP21 |
| 560 | AA729034 | ODC1 | ornithine decarboxylase 1 |
| 561 | AF012549 | ODF2 | outer dense fibre of sperm tails 2 |
| 562 | AA889218 | OGN | osteoglycin (osteoinductive |
| | | | factor, mimecan) |
| 563 | AA922747 | OXR1 | oxidation resistance 1 |
| 564 | M37721 | PAM | peptidylglycine alpha-amidating |
| | | | monooxygenase |
| 565 | X76770 | PAP | poly(A) polymerase |
| 566 | U02020 | PBEF | pre-B-cell colony-enhancing |
| | | | factor |
| 567 | AA626775 | PCDHA5 | protocadherin alpha 5 |
| 568 | D84307 | PCYT2 | phosphate cytidylyltransferase 2, |
| | | | ethanolamine |
| 569 | AA004890 | PDCD8 | programmed cell death 8 |
| | | | (apoptosis-inducing factor) |
| 570 | AA400893 | PDE1A | phosphodiesterase 1A, |
| | | | calmodulin-dependent |
| 571 | AI192411 | PDGFRA | platelet-derived growth factor |
| | | | receptor, alpha polypeptide |
| 572 | C05229 | PDK4 | pyruvate dehydrogenase kinase, |
| | | | isoenzyme 4 |
| 573 | U79296 | PDX1 | Pyruvate dehydrogenase complex, |
| | | | lipoyl-containing component X; |
| | | | E3-binding protein |
| 574 | J00123 | PENK | proenkephalin |
| 575 | AF048755 | PEX13 | peroxisome biogenesis factor 13 |
| 576 | D25328 | PFKP | phosphofructokinase, platelet |
| 577 | W58700 | PHKB | phosphorylase kinase, beta |
| 578 | AA057243 | PHRET1 | PH domain containing protein in |
| | | | retina 1 |
| 579 | AA515710 | PIGN | phosphatidylinositol glycan, class N |
| 580 | AA634825 | PINK1 | PTEN induced putative kinase 1 |
| 581 | U09117 | PLCD1 | phospholipase C, delta 1 |
| 582 | AA777648 | PMP22 | peripheral myelin protein 22 |
| 583 | AF023455 | PPEF1 | protein phosphatase, EF hand |
| | | | calcium-binding domain 1 |
| 584 | AF034803 | PPFIBP2 | PTPRF interacting protein, |
| | | | binding protein 2 (liprin beta 2) |
| 585 | Z50749 | PPP1R7 | protein phosphatase 1, regulatory |
| | | | subunit 7 |
| 586 | M60484 | PPP2CB | protein phosphatase 2 (formerly |
| | | | 2A), catalytic subunit, beta |
| | | | isoform |
| 587 | U37352 | PPP2R5C | protein phosphatase 2, regulatory |
| | | | subunit B (B56), gamma isoform |
| 588 | AI299911 | PPP3CA | protein phosphatase 3 (formerly |
| | | | 2B), catalytic subunit, alpha |
| | | | isoform (calcineurin A alpha) |
| 589 | N29328 | PPP4R1 | protein phosphatase 4, regulatory |
| | | | subunit 1 |
| 590 | X75756 | PRKCM | protein kinase C, mu |
| 591 | AI357236 | PRM1 | protamine 1 |
| 592 | X07862 | PRM2 | protamine 2 |
| 593 | AI242370 | PRND | prion gene complex, downstream |
| 594 | U51990 | PRP18 | pre-mRNA splicing factor similar |
| | | | to S. cerevisiae Prp18 |
| 595 | Y00971 | PRPS2 | phosphoribosyl pyrophosphate |
| | | | synthetase 2 |
| 596 | D87258 | PRSS11 | protease, serine, 11 (IGF binding) |
| 597 | M61900 | PTGDS | prostaglandin D synthase gene |
| 598 | M57399 | PTN | pleiotrophin (heparin binding |
| | | | growth factor 8, neurite growth- |
| | | | promoting factor 1) |
| 599 | W84417 | RANBP9 | RAN binding protein 9 |
| 600 | AA635922 | RANGAP1 | Ran GTPase activating protein 1 |
| 601 | AB008109 | RGS5 | regulator of G-protein signalling 5 |
| 602 | AA778308 | RNASE1 | ribonuclease, RNase A family, 1 |
| | | | (pancreatic) |
| 603 | AA854469 | RNF6 | ring finger protein (C3H2C3 type) 6 |
| 604 | AI095724 | RPL17 | ribosomal protein L17 |
| 605 | AF056929 | SARCOSIN | sarcomeric muscle protein |
| 606 | Y13647 | SCD | stearoyl-CoA desaturase (delta-9- |
| | | | desaturase) |
| 607 | AJ224677 | SCRG1 | scrapie responsive protein 1 |
| 608 | T36260 | SEC23B | Sec23 (S. cerevisiae) homolog B |
| 609 | AA401227 | SEC31B-1 | Secretory pathway component |
| | | | Sec31B-1 |
| 610 | AA703667 | SEC8 | secretory protein, SEC8 |
| 611 | AI026695 | SENP1 | Sentrin/SUMO-specific protease |
| 612 | Z11793 | SEPP1 | selenoprotein P, plasma, 1 |
| 613 | AF042081 | SH3BGRL | SH3 domain binding glutamic |
| | | | acid-rich protein like |
| 614 | AF036269 | SH3GL3 | SH3-domain GRB2-like 3 |
| 615 | T35854 | SIAH2 | seven in absentia (Drosophila) |
| | | | homolog 2 |
| 616 | N53491 | SIRT3 | sir2-like 3 |
| 617 | AA639599 | SLC12A2 | solute carrier family 12 |
| | | | (sodium/potassium/chloride |
| | | | transporters), member 2 |
| 618 | N30856 | SLC19A2 | solute carrier family 19 (thiamine |
| | | | transporter), member 2 |
| 619 | M55531 | SLC2A5 | solute carrier family 2 (facilitated |
| | | | glucose transporter), member 5 |
| 620 | AA838741 | SLC35A1 | Solute carrier family 35 (CMP- |
| | | | sialic acid transporter), member 1 |
| 621 | AA758636 | SMAP | Thyroid hormone receptor |
| | | | coactivating protein |
| 622 | M88163 | SMARCA1 | SWI/SNF related, matrix |
| | | | associated, actin dependent |
| | | | regulator of chromatin, subfamily |
| | | | a, member 1 |
| 623 | W70141 | SMARCA3 | SWI/SNF related, matrix |
| | | | associated, actin dependent |
| | | | regulator of chromatin, subfamily |
| | | | a, member 3 |
| 624 | AI222903 | SMARCD2 | SWI/SNF related, matrix |
| | | | associated, actin dependent |
| | | | regulator of chromatin, subfamily |
| | | | d, member 2 |
| 625 | AI351686 | SMOC1 | secreted modular calcium-binding |
| | | | protein 1 |
| 626 | AA946930 | SNRPG | small nuclear ribonucleoprotein |
| | | | polypeptide G |
| 627 | W56480 | SOS1 | son of sevenless (Drosophila) |
| | | | homolog 1 |
| 628 | Z46629 | SOX9 | SRY (sex determining region Y)- |
| | | | box 9 (campomelic dysplasia, |
| | | | autosomal sex-reversal) |
| 629 | AA760720 | SPAG6 | sperm associated antigen 6 |
| 630 | AI459767 | SPARCL1 | SPARC-like 1 (mast9, hevin) |
| 631 | AA779272 | SPINK2 | serine protease inhibitor, Kazal |
| | | | type, 2 (acrosin-trypsin inhibitor) |
| 632 | M61199 | SSFA2 | sperm specific antigen 2 |
| 633 | AI024234 | SSTK | Serine/threonine protein kinase |
| | | | SSTK |
| 634 | U17280 | STAR | steroidogenic acute regulatory |
| | | | protein |
| 635 | U14550 | STHM | sialyltransferase |
| 636 | L77564 | STK22B | serine/threonine kinase 22B |
| | | | (spermiogenesis associated) |
| 637 | AA935437 | STRIN | STRIN protein |
| 638 | H10341 | SULTX3 | sulfotransferase-related protein |
| 639 | AA643682 | SUV39H2 | Suppressor of variegation 3-9 |
| | | | (Drosophila) homolog 2; |
| | | | hypothetical protein FLJ23414 |
| 640 | Z21437 | TAF2G | TATA box binding protein |
| | | | (TBP)-associated factor, RNA |
| | | | polymerase II, G, 32 kD |
| 641 | AI093734 | TAZ | Transcriptional co-activator with |
| | | | PDZ-binding motif (TAZ) |
| 642 | AA628669 | TBL2 | transducin (beta)-like 2 |
| 643 | AI243203 | TEX14 | Testis expressed sequence 14 |
| 644 | S95936 | TF | transferrin |
| 645 | AA573143 | TIMP2 | tissue inhibitor of |
| | | | metalloproteinase 2 |
| 646 | AI086204 | TM4SF6 | transmembrane 4 superfamily |
| | | | member 6 |
| 647 | U81006 | TM9SF2 | transmembrane 9 superfamily |
| | | | member 2 |
| 648 | L01042 | TMF1 | TATA element modulatory factor 1 |
| 649 | X64559 | TNA | tetranectin (plasminogen-binding |
| | | | protein) |
| 650 | X07948 | TNP1 | transition protein 1 (during |
| | | | histone to protamine replacement) |
| 651 | J04088 | TOP2A | topoisomerase (DNA) II alpha |
| | | | (170 kD) |
| 652 | U54831 | TOP2B | topoisomerase (DNA) II beta |
| | | | (180 kD) |
| 653 | AA913471 | TOPK | PDZ-binding kinase; T-cell |
| | | | originated protein kinase |
| 654 | X66397 | TPR | translocated promoter region (to |
| | | | activated MET oncogene) |
| 655 | M25532 | TPX1 | testis specific protein 1 (probe H4 |
| | | | p3) |
| 656 | X63679 | TRAM | translocating chain-associating |
| | | | membrane protein |
| 657 | AF064801 | TRC8 | patched related protein |
| | | | translocated in renal cancer |
| 658 | AI346969 | TRIM14 | Tripartite motif-containing 14 |
| 659 | AF065388 | TSPAN | tetraspan 1 |
| 660 | AA432312 | TSPYL | TSPY-like |
| 661 | AA456299 | T-STAR | Sam68-like phosphotyrosine |
| | | | protein, T-STAR |
| 662 | X69490 | TTN | titin |
| 663 | AA709190 | TUBA2 | tubulin, alpha 2 |
| 664 | X02308 | TYMS | thymidylate synthetase |
| 665 | AI344684 | UBE2N | ubiquitin-conjugating enzyme |
| | | | E2N (homologous to yeast |
| | | | UBC13) |
| 666 | AA416852 | UBL3 | ubiquitin-like 3 |
| 667 | N44888 | UPF3A | similar to yeast Upf3, variant A |
| 668 | AA116022 | USP18 | ubiquitin specific protease 18 |
| 669 | AA846445 | USP6 | ubiquitin specific protease 6 (Tre- |
| | | | 2 oncogene) |
| 670 | BG028760 | USP7 | ubiquitin specific protease 7 |
| | | | (herpes virus-associated) |
| 671 | T29210 | UTRN | utrophin (homologous to |
| | | | dystrophin) |
| 672 | AI018129 | VAMP4 | vesicle-associated membrane |
| | | | protein 4 |
| 673 | D87459 | WASF1 | WAS protein family, member 1 |
| 674 | S69790 | WASF3 | WAS protein family, member 3 |
| 675 | AA364135 | WDR10 | WD repeat domain 10 |
| 676 | AA160764 | WHSC1 | Wolf-Hirschhorn syndrome |
| | | | candidate 1 |
| 677 | X51630 | WT1 | Wilms tumor 1 |
| 678 | W55933 | WW45 | WW Domain-Containing Gene |
| 679 | N66453 | XPC | xeroderma pigmentosum, |
| | | | complementation group C |
| 680 | D83407 | ZAKI4 | Down syndrome critical region |
| | | | gene 1-like 1 |
| 681 | M92843 | ZFP36 | zinc finger protein homologous to |
| | | | Zfp-36 in mouse |
| 682 | X84801 | ZNF165 | zinc finger protein 165 |
| 683 | AF017433 | ZNF213 | zinc finger protein 213 |
| 684 | AA703988 | ZNF259 | zinc finger protein 259 |
| 685 | AA897714 | ZNF6 | Zinc finger protein 6 (CMPX1) |
| 686 | U54996 | ZW10 | ZW10 (Drosophila) homolog, |
| | | | centromere/kinetochore protein |
| 687 | AA936961 | LOC57032 | similar to acetyl-coenzyme A |
| | | | synthetase |
| 688 | AA234377 | CL25022 | hypothetical protein |
| 689 | N35437 | DJ1181N3.1 | hypothetical protein dJ1181N3.1 |
| 690 | Z20328 | DKFZp434C0328 | hypothetical protein |
| | | | DKFZp434C0328 |
| 691 | H19830 | DKFZP434G156 | hypothetical protein |
| | | | DKFZp434G156 |
| 692 | AI127752 | DKFZP434I092 | DKFZP434I092 protein |
| 693 | T65389 | DKFZP434J214 | DKFZP434J214 protein |
| 694 | AA284134 | DKFZP434L243 | DKFZP434L243 protein |
| 695 | AI192351 | DKFZP564B167 | DKFZP564B167 protein |
| 696 | AA865478 | DKFZP564J0863 | DKFZP564J0863 protein |
| 697 | AI306435 | DKFZP586A0522 | DKFZP586A0522 protein |
| 698 | AA709155 | FLJ10134 | hypothetical protein FLJ10134 |
| 699 | AA582581 | FLJ10159 | hypothetical protein FLJ10159 |
| 700 | AI076154 | FLJ10283 | hypothetical protein FLJ10283 |
| 701 | AA759066 | FLJ10392 | hypothetical protein FLJ10392 |
| 702 | AA452368 | FLJ10582 | hypothetical protein FLJ10582 |
| 703 | U69201 | FLJ10761 | hypothetical protein FLJ10761 |
| 704 | AA418149 | FLJ10850 | hypothetical protein FLJ10850 |
| 705 | AA775271 | FLJ10914 | hypothetical protein FLJ10914 |
| 706 | AA293776 | FLJ10921 | hypothetical protein FLJ10921 |
| 707 | AI221110 | FLJ10980 | hypothetical protein FLJ10980 |
| 708 | AA634293 | FLJ11088 | hypothetical protein FLJ11088 |
| 709 | D81610 | FLJ11109 | hypothetical protein FLJ11109 |
| 710 | AA056538 | FLJ11210 | hypothetical protein FLJ11210 |
| 711 | AA781142 | FLJ11307 | hypothetical protein FLJ11307 |
| 712 | AA214211 | FLJ13110 | hypothetical protein FLJ13110 |
| 713 | AI147953 | FLJ20010 | hypothetical protein |
| 714 | C00491 | FLJ20121 | hypothetical protein FLJ20121 |
| 715 | AK024920 | FLJ20152 | hypothetical protein |
| 716 | AA634416 | FLJ20425 | hypothetical protein FLJ20425 |
| 717 | AA809070 | FLJ20535 | hypothetical protein FLJ20535 |
| 718 | H20535 | FLJ21324 | hypothetical protein FLJ21324 |
| 719 | AI346388 | FLJ21347 | hypothetical protein FLJ21347 |
| 720 | AI016734 | FLJ22104 | hypothetical protein FLJ22104 |
| 721 | AA677445 | H41 | hypothetical protein |
| 722 | AA126461 | HSA272196 | hypothetical protein, clone |
| | | | 2746033 |
| 723 | AI003803 | HSD-3.1 | hypothetical protein |
| 724 | AI300283 | IMPACT | hypothetical protein IMPACT |
| 725 | D38521 | KIAA0077 | KIAA0077 protein |
| 726 | D86984 | KIAA0231 | KIAA0231 protein |
| 727 | D87438 | KIAA0251 | KIAA0251 protein |
| 728 | D87465 | KIAA0275 | KIAA0275 gene product |
| 729 | AF007170 | KIAA0452 | DEME-6 protein |
| 730 | AA910738 | KIAA0579 | KIAA0579 protein |
| 731 | N30392 | KIAA0608 | KIAA0608 protein |
| 732 | AB014534 | KIAA0634 | KIAA0634 protein |
| 733 | AI167680 | KIAA0643 | Homo sapiens cDNA FLJ13257 |
| | | | fis, clone OVARC1000846, |
| | | | weakly similar to NUCLEOLIN |
| 734 | AA506972 | KIAA0668 | KIAA0668 protein |
| 735 | AA665890 | KIAA0729 | KIAA0729 protein |
| 736 | N49366 | KIAA0737 | KIAA0737 gene product |
| 737 | H09503 | KIAA0740 | KIAA0740 gene product |
| 738 | AF052170 | KIAA0750 | KIAA0750 gene product |
| 739 | AA234129 | KIAA0863 | KIAA0863 protein |
| 740 | AA399583 | KIAA0874 | KIAA0874 protein |
| 741 | H03641 | KIAA0914 | KIAA0914 gene product |
| 742 | AI253232 | KIAA0996 | KIAA0996 protein |
| 743 | AA339816 | KIAA1028 | KIAA1028 protein |
| 744 | AI187395 | KIAA1053 | KIAA1053 protein |
| 745 | AA056734 | KIAA1110 | KIAA1110 protein |
| 746 | AI217997 | KIAA1128 | KIAA1128 protein |
| 747 | AA037467 | KIAA1165 | hypothetical protein KIAA1165 |
| 748 | AA994997 | KIAA1223 | KIAA1223 protein |
| 749 | W68261 | KIAA1327 | KIAA1327 protein |
| 750 | AA781940 | KIAA1336 | KIAA1336 protein |
| 751 | AI082425 | KIAA1430 | KIAA1430 protein |
| 752 | AI243817 | KIAA1494 | Homo sapiens cDNA: FLJ23073 |
| | | | fis, clone LNG05726 |
| 753 | AA824313 | KIAA1505 | KIAA1505 protein |
| 754 | D59339 | KIAA1529 | Homo sapiens mRNA; cDNA |
| | | | DKFZp434I2420 (from clone |
| | | | DKFZp434I2420) |
| 755 | AA044905 | KIAA1596 | KIAA1596 protein |
| 756 | T34177 | LOC51255 | hypothetical protein |
| 757 | AA776749 | LOC57821 | hypothetical protein LOC57821 |
| 758 | R00068 | PRO1580 | hypothetical protein PRO1580 |
| 759 | AI302506 | PRO1912 | PRO1912 protein |
| 760 | AF113020 | PRO2463 | PRO2463 protein |
| 761 | AI218544 | FLJ20425 | hypothetical protein FLJ20425 |
| 762 | AI214973 | KIAA1223 | KIAA1223 protein |
| 763 | AI215074 | | Homo sapiens cDNA FLJ11095 |
| | | | fis, clone PLACE1005374 |
| 764 | AA587860 | | Homo sapiens cDNA FLJ11205 |
| | | | fis, clone PLACE1007843 |
| 765 | AA043562 | | Homo sapiens cDNA FLJ11667 |
| | | | fis, clone HEMBA1004697 |
| 766 | AI277493 | | Homo sapiens cDNA FLJ11756 |
| | | | fis, clone HEMBA1005595, |
| | | | weakly similar to DYNEIN |
| | | | HEAVY CHAIN, CYTOSOLIC |
| 767 | AI078809 | | Homo sapiens cDNA FLJ12627 |
| | | | fis, clone NT2RM4001813, |
| | | | weakly similar to LECTIN BRA-2 |
| 768 | AI028392 | | Homo sapiens cDNA FLJ13229 |
| | | | fis, clone OVARC1000106 |
| 769 | AA830551 | | Homo sapiens cDNA FLJ13848 |
| | | | fis, clone THYRO1000855 |
| 770 | AA853955 | | Homo sapiens cDNA FLJ13992 |
| | | | fis, clone Y79AA1002139, |
| | | | weakly similar to DNAJ |
| | | | PROTEIN HOMOLOG 1 |
| 771 | AA320463 | | Homo sapiens cDNA: FLJ21127 |
| | | | fis, clone CAS06212 |
| 772 | AA393838 | | Homo sapiens cDNA: FLJ21849 |
| | | | fis, clone HEP01928 |
| 773 | AA400674 | | Homo sapiens cDNA: FLJ21962 |
| | | | fis, clone HEP05564 |
| 774 | AA148493 | | Homo sapiens cDNA: FLJ22300 |
| | | | fis, clone HRC04759 |
| 775 | AA411157 | | Homo sapiens cDNA: FLJ22448 |
| | | | fis, clone HRC09541 |
| 776 | AA631197 | | Homo sapiens cDNA: FLJ22477 |
| | | | fis, clone HRC10815 |
| 777 | T65582 | | Homo sapiens cDNA: FLJ22637 |
| | | | fis, clone HSI06677 |
| 778 | AI192127 | | Homo sapiens cDNA: FLJ22712 |
| | | | fis, clone HSI13435 |
| 779 | AA148566 | | Homo sapiens cDNA: FLJ22790 |
| | | | fis, clone KAIA2176, highly |
| | | | similar to HUMPMCA Human |
| | | | plasma membrane calcium- |
| | | | pumping ATPase (PMCA4) |
| | | | mRNA |
| 780 | AA633352 | | Homo sapiens cDNA: FLJ23067 |
| | | | fis, clone LNG04993 |
| 781 | AI084531 | | Homo sapiens cDNA: FLJ23093 |
| | | | fis, clone LNG07264 |
| 782 | AA450190 | | Homo sapiens cDNA: FLJ23316 |
| | | | fis, clone HEP12031 |
| 783 | AA975521 | | Homo sapiens cDNA: FLJ23518 |
| | | | fis, clone LNG04878 |
| 784 | AI097058 | | Homo sapiens cDNA: FLJ23538 |
| | | | fis, clone LNG08010, highly |
| | | | similar to BETA2 Human MEN1 |
| | | | region clone epsilon/beta mRNA |
| 785 | AA405953 | | Homo sapiens chromosome 11 |
| | | | unknown mRNA sequence |
| 786 | N32181 | | Homo sapiens clone 25056 |
| | | | mRNA sequence |
| 787 | AA262802 | | Homo sapiens clone SP329 |
| | | | unknown mRNA |
| 788 | AA293837 | | Homo sapiens GKAP42 |
| | | | (FKSG21) mRNA, complete cds |
| 789 | AA970955 | | Homo sapiens mRNA; cDNA |
| | | | DKFZp434B0610 (from clone |
| | | | DKFZp434B0610); partial cds |
| 790 | AA843455 | | Homo sapiens mRNA; cDNA |
| | | | DKFZp434E232 (from clone |
| | | | DKFZp434E232) |
| 791 | AA421199 | | Homo sapiens mRNA; cDNA |
| | | | DKFZp434L0217 (from clone |
| | | | DKFZp434L0217); partial cds |
| 792 | AA393597 | | Homo sapiens mRNA; cDNA |
| | | | DKFZp434P2072 (from clone |
| | | | DKFZp434P2072); partial cds |
| 793 | AA976808 | | Homo sapiens mRNA; cDNA |
| | | | DKFZp564C046 (from clone |
| | | | DKFZp564C046) |
| 794 | AI280901 | | Homo sapiens mRNA; cDNA |
| | | | DKFZp564D016 (from clone |
| | | | DKFZp564D016) |
| 795 | AA443685 | | Homo sapiens mRNA; cDNA |
| | | | DKFZp564H142 (from clone |
| | | | DKFZp564H142) |
| 796 | N41310 | | Homo sapiens mRNA; cDNA |
| | | | DKFZp564P046 (from clone |
| | | | DKFZp564P046) |
| 797 | AI299718 | | Homo sapiens mRNA; cDNA |
| | | | DKFZp586B1922 (from clone |
| | | | DKFZp586B1922) |
| 798 | AA280818 | | Homo sapiens mRNA; cDNA |
| | | | DKFZp586G2222 (from clone |
| | | | DKFZp586G2222) |
| 799 | AI150152 | | Homo sapiens PAC clone RP5- |
| | | | 981O7 from 7q34-q36 |
| 800 | AI016755 | | Homo sapiens ropporin mRNA, |
| | | | complete cds |
| 801 | AI014769 | | Homo sapiens TRAF4 associated |
| | | | factor 1 mRNA, partial cds |
| 802 | AA004698 | | Homo sapiens ubiquitin-like |
| | | | fusion protein mRNA, complete |
| | | | cds |
| 803 | AA431698 | | Human DNA sequence from |
| | | | clone 1068E13 on chromosome |
| | | | 20p11.212.3. Contains two |
| | | | putative novel genes, the gene for |
| | | | a novel protein similar to bovine |
| | | | SCP2 (Sterol Carrier Protein 2) |
| | | | and part of HSD17B4 |
| | | | (hydroxysteroid (17-beta) |
| | | | dehydrogenase 4), an EEF1A1 ( |
| 804 | AA126472 | | Human DNA sequence from |
| | | | clone 747H23 on chromosome |
| | | | 6q135. Contains the 3′ part of the |
| | | | ME1 gene for malic enzyme 1, |
| | | | soluble (NADP-dependent malic |
| | | | enzyme, malate oxidoreductase, |
| | | | EC 1.1.1.40), a novel gene and |
| | | | the 5′ part of the gene for N- |
| | | | acetylglucosamine |
| 805 | AA651872 | | Human DNA sequence from |
| | | | clone RP12G14 on chromosome |
| | | | 6q24.1-25.2. Contains the 5′ end |
| | | | of the gene for a novel cyclophilin |
| | | | type peptidyl-prolyl cis-trans |
| | | | isomerase, a novel gene, an |
| | | | RPS18 (40S Ribosomal protein |
| | | | S18) pseudogene, the 3′ end of the |
| | | | KATNA1 gen |
| 806 | A25270 | | IFN-gamma antagonist cytokine |
| 807 | AA650281 | | Likely ortholog of mouse tumor |
| | | | necrosis-alpha-induced adipose- |
| | | | related protein |
| 808 | AI015633 | | Solute carrier family 26, member 8 |
| 809 | N47682 | KIAA1673 | ESTs |
| 810 | AA578684 | KIAA1674 | ESTs |
| 811 | Z21254 | KIAA1771 | ESTs, Weakly similar to |
| | | | unnamed protein product |
| | | | [H. sapiens] |
| 812 | R61253 | KIAA1877 | ESTs |
| 813 | W67209 | KIAA0251 | ESTs, Moderately similar to |
| | | | p53 regulated PA26-T2 nuclear |
| | | | protein [H. sapiens] |
| 814 | AA609891 | | EST |
| 815 | W86641 | | EST |
| 816 | AA815470 | | EST |
| 817 | AA992324 | | EST |
| 818 | AA446449 | | EST |
| 819 | AI004873 | | EST |
| 820 | AI093982 | | EST |
| 821 | AA393055 | | ESTs |
| 822 | AI168436 | | ESTs |
| 823 | AA809072 | | ESTs |
| 824 | AA926704 | | ESTs |
| 825 | AI183575 | | ESTs |
| 826 | AA121865 | | ESTs |
| 827 | AA725836 | | ESTs |
| 828 | AA621076 | | ESTs |
| 829 | AI018394 | | ESTs |
| 830 | AA885079 | | ESTs |
| 831 | AI148659 | | ESTs |
| 832 | AA460513 | | ESTs |
| 833 | AA758005 | | ESTs |
| 834 | AA868233 | | ESTs |
| 835 | AA488768 | | ESTs |
| 836 | AA496024 | | ESTs |
| 837 | AA496252 | | ESTs |
| 838 | AI339257 | | ESTs |
| 839 | T64080 | | ESTs |
| 840 | AA844729 | | ESTs |
| 841 | AI041148 | | ESTs |
| 842 | AA813319 | | ESTs |
| 843 | AI138555 | | ESTs |
| 844 | AA633536 | | ESTs |
| 845 | AA688025 | | ESTs |
| 846 | U51712 | | ESTs |
| 847 | N50822 | | ESTs |
| 848 | R38569 | | ESTs |
| 849 | AA889533 | | ESTs |
| 850 | AA629398 | | ESTs |
| 851 | AA628190 | | ESTs |
| 852 | AI041289 | | ESTs |
| 853 | AI204513 | | ESTs |
| 854 | AA001410 | | ESTs |
| 855 | AI027500 | | ESTs |
| 856 | AA658107 | | ESTs |
| 857 | AA923244 | | ESTs |
| 858 | AA723819 | | ESTs |
| 859 | AA437069 | | ESTs |
| 860 | AA400934 | | ESTs |
| 861 | M32093 | | ESTs |
| 862 | AA262466 | | ESTs |
| 863 | AA897137 | | ESTs |
| 864 | AA446184 | | ESTs |
| 865 | AA036631 | | ESTs |
| 866 | H86103 | | ESTs |
| 867 | AA401541 | | ESTs |
| 868 | H05826 | | ESTs |
| 869 | AA406039 | | ESTs |
| 870 | AA448082 | | ESTs |
| 871 | AA446064 | | ESTs |
| 872 | H81935 | | ESTs |
| 873 | AA889152 | | ESTs |
| 874 | AI127656 | | ESTs |
| 875 | AI033705 | | ESTs |
| 876 | AI138800 | | ESTs |
| 877 | AI183653 | | ESTs |
| 878 | AA969732 | | ESTs |
| 879 | AI024328 | | ESTs |
| 880 | AA913732 | | ESTs |
| 881 | AA397520 | | ESTs |
| 882 | AI025509 | | ESTs |
| 883 | AA382504 | | ESTs |
| 884 | AI341170 | | ESTs |
| 885 | AA909257 | | ESTs |
| 886 | AA812677 | | ESTs |
| 887 | AA416673 | | ESTs |
| 888 | AA972840 | | ESTs |
| 889 | W31789 | | ESTs |
| 890 | AI261804 | | ESTs |
| 891 | AI091533 | | ESTs |
| 892 | AA991994 | | ESTs |
| 893 | AI024578 | | ESTs |
| 894 | AI040955 | | ESTs |
| 895 | AA953477 | | ESTs |
| 896 | AA846324 | | ESTs |
| 897 | AA417966 | | ESTs |
| 898 | AA150262 | | ESTs |
| 899 | AA724720 | | ESTs |
| 900 | AI031941 | | ESTs |
| 901 | AA620800 | | ESTs |
| 902 | AA813092 | | ESTs |
| 903 | AA101229 | | ESTs |
| 904 | AA025055 | | ESTs |
| 905 | AA382809 | | ESTs |
| 906 | R60655 | | ESTs, Highly similar to |
| | | | AC005534 2 supported by human |
| | | | ESTs AA412402 [H. sapiens] |
| 907 | AA521265 | | ESTs, Highly similar to |
| | | | AF117065 1 male-specific lethal- |
| | | | 3 homolog 1 [H. sapiens] |
| 908 | D50640 | | ESTs, Highly similar to |
| | | | CN3B_HUMAN CGMP- |
| | | | INHIBITED 3′,5′-CYCLIC |
| | | | PHOSPHODIESTERASE B |
| | | | [H. sapiens] |
| 909 | W44613 | | ESTs, Highly similar to |
| | | | differentially expressed in |
| | | | Fanconi anemia [H. sapiens] |
| 910 | AA400550 | | ESTs, Moderately similar to |
| | | | ALU4_HUMAN ALU |
| | | | SUBFAMILY SB2 SEQUENCE |
| | | | CONTAMINATION WARNING |
| | | | ENTRY [H. sapiens] |
| 911 | AA648782 | | ESTs, Moderately similar to |
| | | | GNPI_HUMAN |
| | | | GLUCOSAMINE-6- |
| | | | PHOSPHATE ISOMERASE |
| | | | [H. sapiens] |
| 912 | AA496122 | | ESTs, Moderately similar to |
| | | | KIAA1165 protein [H. sapiens] |
| 913 | AI039250 | | ESTs, Moderately similar to |
| | | | p60 katanin [H. sapiens] |
| 914 | AI187883 | | ESTs, Weakly similar to actin |
| | | | binding protein MAYVEN |
| | | | [H. sapiens] |
| 915 | AA865734 | | ESTs, Weakly similar to |
| | | | AF141326 1 RNA helicase |
| | | | HDB/DICE1 [H. sapiens] |
| 916 | D20934 | | ESTs, Weakly similar to |
| | | | AF148856 1 unknown |
| | | | [H. sapiens] |
| 917 | AI434204 | | ESTs, Weakly similar to Afg1p |
| | | | [S. cerevisiae] |
| 918 | AA876372 | | ESTs, Weakly similar to |
| | | | ALU1_HUMAN ALU |
| | | | SUBFAMILY J SEQUENCE |
| | | | CONTAMINATION WARNING |
| | | | ENTRY [H. sapiens] |
| 919 | AI150114 | | ESTs, Weakly similar to |
| | | | ALU1_HUMAN ALU |
| | | | SUBFAMILY J SEQUENCE |
| | | | CONTAMINATION WARNING |
| | | | ENTRY [H. sapiens] |
| 920 | AA533191 | | ESTs, Weakly similar to |
| | | | ALU7_HUMAN ALU |
| | | | SUBFAMILY SQ SEQUENCE |
| | | | CONTAMINATION WARNING |
| | | | ENTRY [H. sapiens] |
| 921 | AA885514 | | ESTs, Weakly similar to |
| | | | CAYP_HUMAN |
| | | | CALCYPHOSINE [H. sapiens] |
| 922 | AA960902 | | ESTs, Weakly similar to |
| | | | COXM_HUMAN |
| | | | CYTOCHROME C OXIDASE |
| | | | POLYPEPTIDE VIIB |
| | | | PRECURSO [H. sapiens] |
| 923 | AI336338 | | ESTs, Weakly similar to |
| | | | dJ1108D11.1 [H. sapiens] |
| 924 | AI208582 | | ESTs, Weakly similar to |
| | | | dJ134E15.1 [H. sapiens] |
| 925 | AA927467 | | ESTs, Weakly similar to I38428 |
| | | | T-complex protein 10A |
| | | | [H. sapiens] |
| 926 | AA789329 | | ESTs, Weakly similar to katanin |
| | | | p80 subunit [H. sapiens] |
| 927 | AA453640 | | ESTs, Weakly similar to |
| | | | KCC1_HUMAN |
| | | | CALCIUM/CALMODULIN- |
| | | | DEPENDENT PROTEIN |
| | | | KINASE TYPE I [H. sapiens] |
| 928 | AA744373 | | ESTs, Weakly similar to |
| | | | KIAA1006 protein [H. sapiens] |
| 929 | AA393227 | | ESTs, Weakly similar to |
| | | | KIAA1016 protein [H. sapiens] |
| 930 | AI126471 | | ESTs, Weakly similar to MRJ |
| | | | [H. sapiens] |
| 931 | AA843459 | | ESTs, Weakly similar to PRP2 |
| | | | MOUSE PROLINE-RICH |
| | | | PROTEIN MP-2 PRECURSOR |
| | | | [M. musculus] |
| 932 | R79064 | | ESTs, Weakly similar to |
| | | | putative type III alcohol |
| | | | dehydrogenase [D. melanogaster] |
| 933 | AA708149 | | ESTs, Weakly similar to |
| | | | Similarity to Human ADP/ATP |
| | | | carrier protein [C. elegans] |
| 934 | AA946954 | | ESTs, Weakly similar to |
| | | | testicular condensing enzyme |
| | | | [M. musculus] |
| 935 | AA045194 | | ESTs, Weakly similar to |
| | | | testicular tektin B1-like protein |
| | | | [H. sapiens] |
| 936 | AA223199 | | ESTs, Weakly similar to |
| | | | Unknown gene product |
| | | | [H. sapiens] |
| 937 | AA843452 | | ESTs, Weakly similar to weak |
| | | | similarity to SP: YAD5 CLOAB |
| | | | [C. elegans] |
| 938 | AI224867 | | ESTs, Weakly similar to zinc |
| | | | finger protein [H. sapiens] |
| 939 | AI024879 | | ESTs, Weakly similar to zona- |
| | | | pellucida-binding protein |
| | | | [H. sapiens] |

TABLE 5
Representative up-regulated genes with known
function in testicular seminomas

| | | | |
|---|---|---|---|
| TS | Accession | | |
| Assignment | No. | Symbol | Gene Name |

genes involved in signal transduction pathways

| | | | |
|---|---|---|---|
| 107 | D87116 | MAP2K3 | mitogen-activated protein kinase |
| | | | kinase 3 |
| 97 | AA845512 | KLF4 | Kruppel-like factor 4 (gut) |
| 108 | AA583183 | MAP4K3 | mitogen-activated protein kinase |
| | | | kinase kinase kinase 3 |
| 162 | AA346311 | RAI3 | retinoic acid induced 3 |
| 163 | M29893 | RALA | v-ral simian leukemia viral |
| | | | oncogene homolog A |
| | | | (ras related) |
| 120 | M13228 | MYCN | v-myc avian myelocytomatosis |
| | | | viral related oncogene, |
| | | | neuroblastoma derived |

genes involved in oncogenesis

| | | | |
|---|---|---|---|
| 153 | AF045584 | POV1 | prostate cancer overexpressed |
| | | | gene 1 |
| 147 | M16750 | PIM1 | pim oncogene |
| 148 | U77735 | PIM2 | pim-2 oncogene |
| 225 | AA465240 | VAV2 | vav 2 oncogene |
| 170 | X12949 | RET | ret proto-oncogene |

genes involved in cell cycle

| | | | |
|---|---|---|---|
| 20 | AA682870 | CCND2 | cyclin D2 |
| 25 | M81934 | CDC25B | cell division cycle 25B |

genes involved in cell adhesion and cytoskeleton

| | | | |
|---|---|---|---|
| 92 | Z68228 | JUP | junction plakoglobin |
| 45 | AA128470 | DSP | desmoplakin (DPI, DPII) |
| 26 | X63629 | CDH3 | cadherin 3, type 1, P-cadherin |
| | | | (placental) |
| 96 | U06698 | KIF5A | kinesin family member 5A |

Semi-quantitative RT-PCR

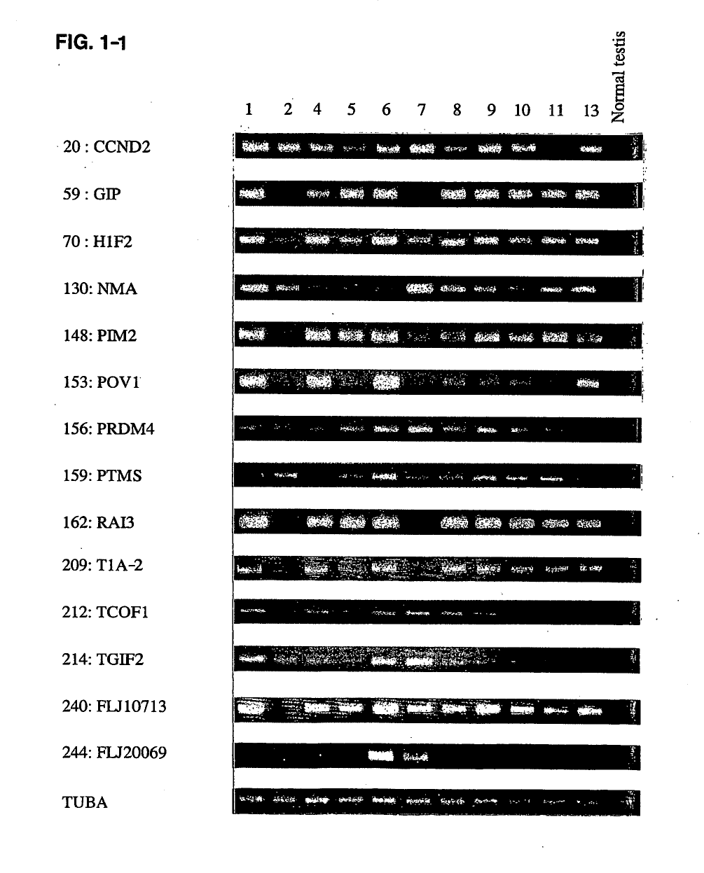

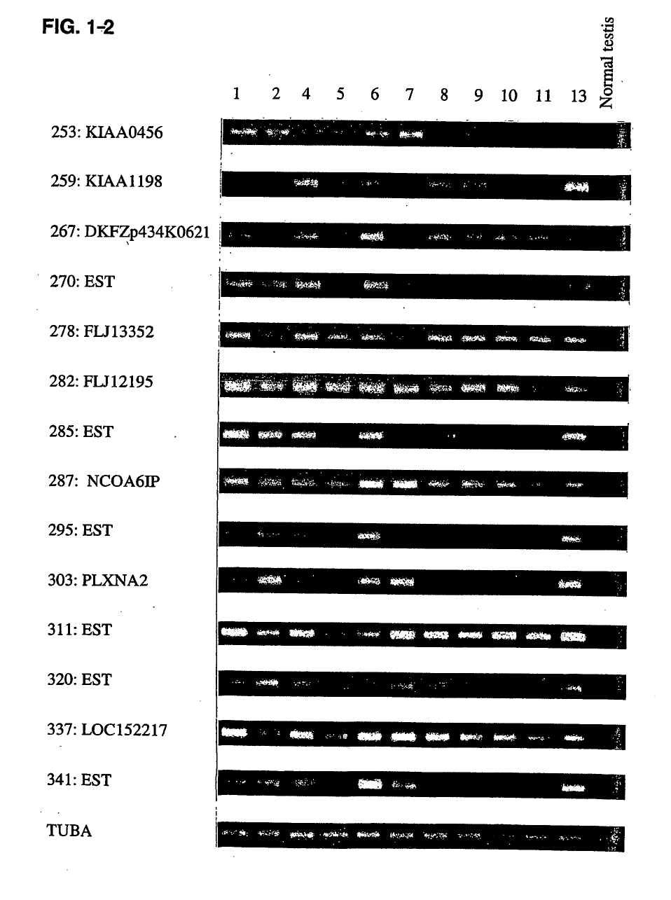

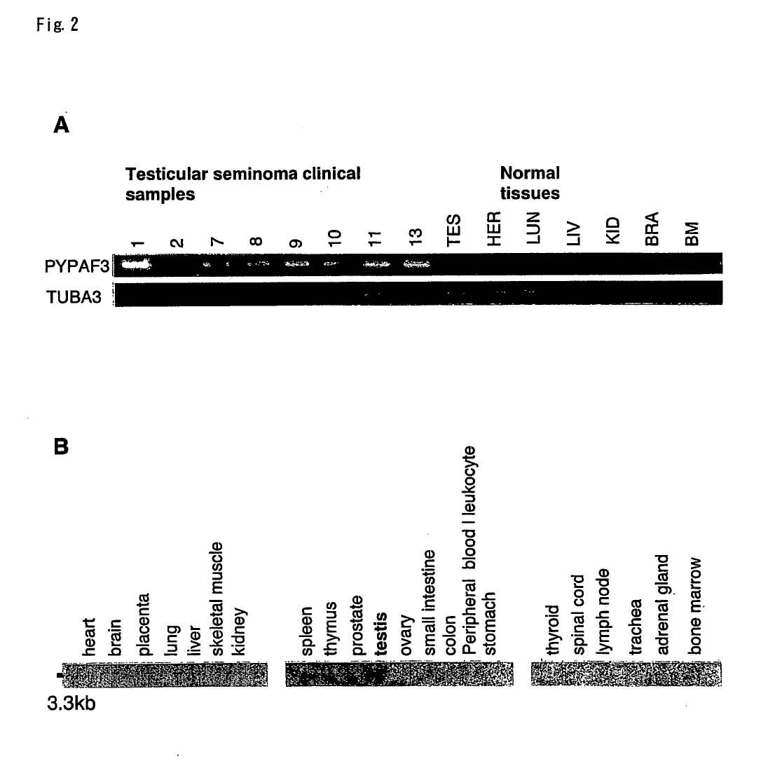

Twenty nine up-regulated genes were selected and their expression levels examined by applying the semi-quantitative RT-PCR experiments. A 3-μg aliquot of aRNA from each sample was reverse-transcribed for single-stranded cDNAs using random primer (Roche) and Superscript II (Life Technologies, Inc.). Each cDNA mixture was diluted for subsequent PCR amplification with the same primer sets that were prepared for the target DNA- or α-tublin-specific reactions. The primer sequences are listed in Table 2. Expression of α-tublin served as an internal control. PCR reactions were optimized for the number of cycles to ensure product intensity within the linear phase of amplification. Comparing the ratios of the expression levels of the 29 up-regulated genes (CCND2, GIP, H1F2, NMA, PIM2, POV1, PRDM4, PTMS, RAI3, PYPAF3, T1A-2, TCOF1, TGIF2, FLJ10713, FLJ20069, KIAA0456, KIAA1198, DKFZp434K0621, EST(270), FLJ13352, FLJ12195, EST(285), NCOA6IP, EST(295), PLXNA2, EST(311), EST(320), LOC152217, EST(341)) whose expression were overexpressed in almost of all informative cases, the results were highly similar to those of the microarray analysis in the great majority of the tested cases (FIG. 1, FIG. 2A).

TABLE 2

Primer Sequence for RT-PCR

| | | | | | |
|---|---|---|---|---|---|
| | | | SEQ | | SEQ |
| TS | | | ID | | ID |
| Assignment | GENE | Forward Primer | NO | Reverse Primer | NO |

| | | | | | |
|---|---|---|---|---|---|
| 20 | CCND2 | 5′-TGATCAGTGTAT | 1 | 5′-GGTCAAGGTGAGTT | 2 |
| | | GCGAAAAGGT-3′ | | TATTGTCCA-3′ | |
| 59 | GIP | 5′-TTGCCATGGACA | 3 | 5′-TTGTCTGATCCAGC | 4 |
| | | AGATTCAC-3′ | | AAGCAG-3′ | |
| 70 | H1F2 | 5′-CGGAACCAAACC | 5 | 5′-CTTCACAGCCTTAG | 6 |
| | | TAAGAAGC-3′ | | CAGCACTT-3′ | |
| 130 | NMA | 5′-CCTCTGCAAACA | 7 | 5′-AAGATGTAGAAGCT | 8 |
| | | GAATCTTG-3′ | | TACATAGGGCA-3′ | |
| 148 | PIM2 | 5′-GGAAATAAGGCT | 9 | 5′-AATAGTGGGTTTCC | 10 |
| | | TGCTGTTTGT-3′ | | ACACATGG-3′ | |
| 153 | POV1 | 5′-CACAACATGCAA | 11 | 5′-TCCTCTAAGACTTG | 12 |
| | | TGTGTCTGTG-3′ | | CAAGCAGC-3′ | |
| 156 | PRDM4 | 5′-CATGAAGGAAAA | 13 | 5′-GTGCAGAAAGAGA | 14 |
| | | CTCATCCG-3′ | | CTCATCCG-3′ | |
| 159 | PTMS | 5′-TCCCACCTAACCT | 15 | 5′-GAAGCGCGACCATT | 16 |
| | | CTGCATC-3′ | | TCTTTA-3′ | |
| 162 | RAI3 | 5′-GGCTGATACTTCT | 17 | 5′-GCCACCACATCTTT | 18 |
| | | CTCATCTTGC-3′ | | ATTGCATAC-3′ | |
| 171 | PYPAF3 | 5′-TGGGGTTCTAAG | 19 | 5′-GTGAGAAAACCAGT | 20 |
| | | ACAAAGAACTG-3′ | | GTCAAATCC-3′ | |
| 209 | T1A-2 | 5′-TGCTGGTGCTATT | 21 | 5′-AAAAGACCGTTTCT | 22 |
| | | TACTGACGTA-3′ | | GACTCTGTG-3′ | |
| 212 | TCOF1 | 5′-AAGTGACCTCCT | 23 | 5′-CACCCTTCCTCCAA | 24 |
| | | CTCCTTCC-3′ | | GTCTTTTAT-3′ | |
| 214 | TGIF2 | 5′-GAACCCAGTGGA | 25 | 5′-TACTGCAGAGACTT | 26 |
| | | TGTAACAGAAC-3′ | | AGCTGGTCC-3′ | |
| 240 | FLJ10713 | 5′-ACTTATAGTCCTG | 27 | 5′-GGCAGGAGAGAAG | 28 |
| | | CGAGTCTGGG-3′ | | AACATCTTG-3′ | |
| 244 | FLJ20069 | 5′-CATCTCCTTTGTT | 29 | 5′-GATCACTGTGGGTC | 30 |
| | | TCGATAGGA-3′ | | TTAAGCAA-3′ | |
| 253 | KIAA0456 | 5′-GGGCTGGTGCAG | 31 | 5′-TCCAACATCTGTTG | 32 |
| | | ATCTACTT-3′ | | AGTGACAGT-3′ | |
| 259 | KIAA1198 | 5′-CACTCAGAATTC | 33 | 5′-GTGATGTGAAGCAA | 34 |
| | | TTACCTCCCCT-3′ | | GGTAGTTCC-3′ | |
| 267 | DKFZp4 | 5′-GCCAAAAATGGC | 35 | 5′-CAGACACGCACTTG | 36 |
| | 34K0621 | TCTCTAGG-3′ | | TGGTTTATT-3′ | |
| 270 | EST | 5′-GTGTCCACTTAG | 37 | 5′-ATCCTTCTTCCTATA | 38 |
| | | AGCCTCACG-3′ | | CTTCCCCC-3′ | |
| 278 | FLJ13352 | 5′-TTTAATCAGGCC | 39 | 5′-GGGGTATAGAAATG | 40 |
| | | CTGTCTGC-3′ | | GAATGGAGA-3′ | |
| 282 | FLJ12195 | 5′-CTGGAAGAAGAA | 41 | 5′-GGTTGCTGAGATTT | 42 |
| | | GGAACAGGTCT-3′ | | TATCTGTGG-3′ | |
| 285 | EST | 5′-CAAATGCTCTGC | 43 | 5′-CATGAATGAGCCTG | 44 |
| | | TTTGTACTCCT-3′ | | AAATAGTCC-3′ | |
| 287 | NCOA6IP | 5′-CGGGAGGATTGT | 45 | 5′-ACTTCTCATGAGTT | 46 |
| | | AAGATACTGTG-3′ | | CAGCCTCAG-3′ | |
| 295 | EST | 5′-GTAGATGTGGGG | 47 | 5′-TTTAAAGTCACCTT | 48 |
| | | ACAACAGAGAG-3′ | | AGGTTGGGG-3′ | |
| 303 | PLXNA2 | 5′-GTTTTTGTGGGG | 49 | 5′-GGAGGAAGTAGCT | 50 |
| | | ACTAAGAGTG-3′ | | AGAAGCTAAG-3′ | |
| 311 | EST | 5′-CTTTTCCCACAAG | 51 | 5′-CTGGTGTAATCAGA | 52 |
| | | AACCATTTC-3′ | | CACCACGTA-3′ | |
| 320 | EST | 5′-CTCATCTGTACCC | 53 | 5′-CTAAAGTCTCCCAG | 54 |
| | | TCACTGGGAT-3′ | | TTTCCCCT-3′ | |
| 337 | LOC152 | 5′-AAGCCAGAGAGC | 55 | 5′-CGGTATTCTTAACA | 56 |
| | 217 | CTTTCCTC-3′ | | CATCTTGCC-3′ | |
| 341 | EST | 5′-ACCTAACGTTTGT | 57 | 5′-AGGTTGGAAGATCC | 58 |
| | | GCCTTATGTG-3′ | | ATTTCCTT-3′ | |
| | TUBA | 5′-CTTGGGTCTGTA | 59 | 5′-AAGGATTATGAGGA | 60 |
| | | ACAAAGCATTC-3′ | | GGTTGGTGT-3′ | |
| | β2MG | 5′-TTAGCTGTGCTCG | 61 | 5′-TCACATGGTTCACA | 62 |
| | | CGCTACT-3′ | | CGGCAC-3′ | |

### EXAMPLE 3

### Growth-Inhabitory Effects of siRNA Designed to Reduce Expression of PYPAF3

Through analysis of genome-wide expression profiles by a eDNA microarray, we have applied 5 to isolate novel molecular targets for diagnotic tumor markers, treatments and prevention of testicular germ cell tumor. Among the genes that commonly up-regulated in testicular seminomas, we focused on PYRIN-containing Apaf-1-like protein 3 (PYPAF3(NM_{—}139176)) that were significantly up-regulated in 7 of 8 cases with testicular serninomas, compared to normal human organ including testis, heart, lung, liver, kidney, brain and bone marrow by semi-quantitative RT-PCR analysis. Although we identified PYPAF3 as up-regulated gene in testicular seminona at present (bulid #160), we initially listed this gene up as RMP:RMB5-mediating protein through expression profiles using cDNA microarray representing 23,040 genes that were retrieved from Unigene database (build #131) on Natlonal Center for Biotechnology Information.

Multiple-tissue Northern blot analysis using PYPAF3 cDNA fragment as a probe revealed a transcript of approximately 3.3kb that was expressed only in testis. Immunocytocheminal study revealed PYPAF3 protein was present throughout the cytoplasm. Transfection of small interference RNA (siRNA) of PYPAF3 inhibited the expression of mRNA of PYPAF3 and cell growth of testicular germ cell tumor cells. These findings suggest that PYPAF3 might be involved in tumorigenesis of testicular seminomas, and represents a promising candidate for development of targeted therapy for testicular germ cell tumors.

Cell Lines and Tissue Specimens

COS-7 cells and Tera-2 cells were obtained from the American Type Culture Collection (ATCC, Rockville, Md.). All cell lines were grown in monolayers in appropriate media supplemented with 10% fetal bovine serum and 1% antibiotic/antimycotic solution (Sigma, St. Louis, Mo.), Dulbecco's modified Eagle's medium (Sigma) for COS-7 McCoy's 5A (Invitrogen, Carlsbad Calif.), and maintained at 37° C. in humid air containing 5% CO₂.

Semi-Quantitative RT-PCR

Normal human testis, heart, lung, kidney, liver, brain, and bone marrow poly(A)⁺ RNA were obtained by Clontech (Palo Alto, Calif.). A 3-μg aliquot of amplified RNA from each sample was reverse-transcribed to single-stranded cDNAs using random primer (Roche) and Superscript II reverse transcriptase (Invitrogen). Each single-strand cDNA was diluted for subsequent PCR amplification. Standard RT-PCR procedures were carried out in 20ml volumes of PCR buffer (Takara, Kyoto, Japan), and amplified for 5min at 94° C. for denatureing, followed by 22 (for TUBA3) or 31 (for PYPAF3) cycles of 94° C. for 30sec, 55° C. for 30 sec and 72° C. for 30sec. Primer sequences were as follows: for TUBA3, forward 5′-CTTGGGTCTGTAACAAAGCATTC-3′(SEQ ID NO:59), and reverse 5′-AAGGATTATGAGGAGGTTGGTGT-3′(SEQ ID NO:60); for PYPAF3, forward 5′-TGGGGTTCTAAGACAAAGAACTG-3′(SEQ ID NO:19), and reverse 5′-GTGAGAAAACCAGTGTCAAATCC-3′(SEQ ID NO:20).

Northern Blot Analysis

Human multiple-tissue blots (Clontech) were hybridized with a ³²P-labeled PYPAF3 cDNA fragment as a probe. The cDNA was prepared by RT-PCR as described above. Pre-hybridization, hybridization and washing were performed according to the supplier's recommendations. The blots were autoradiographed with intensifying screens at −80° C. for 7 days.

Immunocytocheminal Staining

The entire coding region of PYPAF3 was amplified by RT-PCR using forward primer 5′-CGCGGATCCCACTATGACATCGCCCCAGC-3′(SEQ ID NO:63) and reverse primer 5′-CCGCTCGAGGCAAAAAAAGTCACAGCACGG-3′(SEQ ID NO:64). After the PCR product was digested with BamH1 and Xho1, it was cloned into an appropriate cloning site of plasmid vector pcDNA3.1-myc/His (Invitrogen). COS7 cells were transfected with pcDNA3. I (+)-PYPAF3-mycIHis mixed with FuGene6 transfection reagent (Roche, Basel, Switzerland). COS7-derived transiently transfectants were washed twice with PBS(−), fixed with 4% paraformnaldehyde solution for 15 min at 4° C., and rendered permeable with PBS(−) containing 0.1% Triton X-100 for 2.5 min. Cells were covered with 3% BSA in PBS(−) for 60 min to block non-specific antibody-binding sites prior to reaction with the primary antibody. PYPAF3 protein was detected with mouse anti-human c-Myc 9E10 antibody (Santa Cruz Biotechnology, Santa Cruz, Calif.) as primary and goat anti-mouse FITC (Jackson ImmunoResearch, West Grove, Pa.) as secondary antibody. Nuclei were counterstained by 4′,6′-diamidine-2′-phenylindole dihydrochloride (Vector Laboratories, Burlingame, Calif.). Fluorescent images were obtained with an Eclipse E800 microscope (Nikon, Tokyo, Japan).

Treatment of Testicular Germ Cell Tumor Cells with Small Interference RNA (siRNA)

Transcription of the U6RNA gene by RNA polymerase III produces short transcripts with uridines at the 3′ ends. We amplified a genomic fragment containing the promoter region of U6RNA by PCR, using primers 5′-TGGTAGCCAAGTGCAGGTTATA-3′(SEQ ID NQ:65), and 5′-CCAAAGGGTTTCTGCAGTTTCA-3′(SEQ ID NO:66) and human placental DNA as a template. The product was purified and cloned into pCR2.1 plasmid vector using a TA cloning kit, according to the supplier's protocol (Invitrogen). The BamHI, XhoI fragment containing U6RNA was purified and cloned into pcDNA3.1(+) between nucleotides 56 and 1257, and the fragment was amplified by PCR using primers 5′-TGCGGATCCAGAGCAGATTGTACTGAGAGT-3′(SEQ ID NO:67) and 5′-CTCTATCTCGAGTGAGGCGGAAAGAACCA-3′(SEQ ID NO:68). The ligated DNA became the template for PCR amplification with primers 5′-TTTAAGCTTGAAGACCATTGGAAAAAAAAAAAAAAAAAAAAAACA-3′(SEQ ID NO:69) and 5′-TTTAAGCTTGAAGACATGGGAAAGAGTGGTCTCA-3′(SEQ ID NO:70). The product was digested with HindHI and subsequently self-ligated to produce a psiU6BX vector plasmid. SiRNA expression vectors against PYPAF3 (psiU6BX-PYPAF3) and control plasmids (psiU6BX-EGFP, psiU6BX-Luciferace) were prepared by cloning double-stranded oligonucleotides following as Table 6 into the BbsI site in the psiU6BX vector. Each siRNA expression vector was transfected with Fugene6 (Roche) into testicular germ cell tumor line Tera-2 which expressed PYPAF3 endogenously. After selection by Geneticin (Invitrogen), cell proliferation was evaluated after two weeks by colony formation assay using Giemsa staining and after one week by Cell Counting Kit-8 (Dojindo, Kumamoto, Japan) (39). A knockdown effect of PYPAF3 mRNA was identified by semi-quantitative RT-PCR.

Confirmation of Expression of PYPAF3in Testicular Seminomas by Semi-Quantitative RT-PCR.

We have been using a cDNA microarray to analyze gene-expression profiles of 23,040 genes in testicular seminomas from 13 patients (12). Among the up-regulated genes, we focused on PYPAF3, which was overexpressed in 7 of 8 informative cases whose signal intensities of the gene were higher than the cut-off in patients with testicular seminomas. Furthermore, we performed semi-quantitative RT-PCR analysis and then confirmed elevated expression of PYPAF3 in 7 of 8 testicular semiunomas, compared to normal human testis, heart, lung, liver, kidney, brain and bone marrow (FIG. 2A).

Multiple-tissue Northern Blot Analysis and Sub-Cellular Localization of PYPAF3 Protein

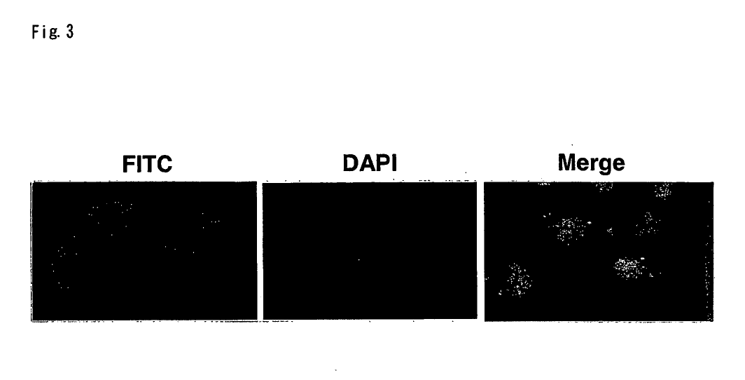

Northern analysis using PYPAF3 cDNA fragment as a probe (see Material and Method) revealed a transcript of approximately 3.3kb that was expressed only in testis (FIG. 2B). Furthermore, to investigate the role of PYPAF3 protein in mammalian cells, we constructed a plasmid to express myc-tagged PYPAF3 protein (see Material and Method). When the plasmid DNA was transiently transfected into COS-7 cells, the tagged PYPAF3 protein was present throughout the cytoplasm of transfected cells (FIG. 3).

Growth-Inhibitory Effects of Small-Interference RNA (siRNA) Designed to Reduce Expression of PYPAF3

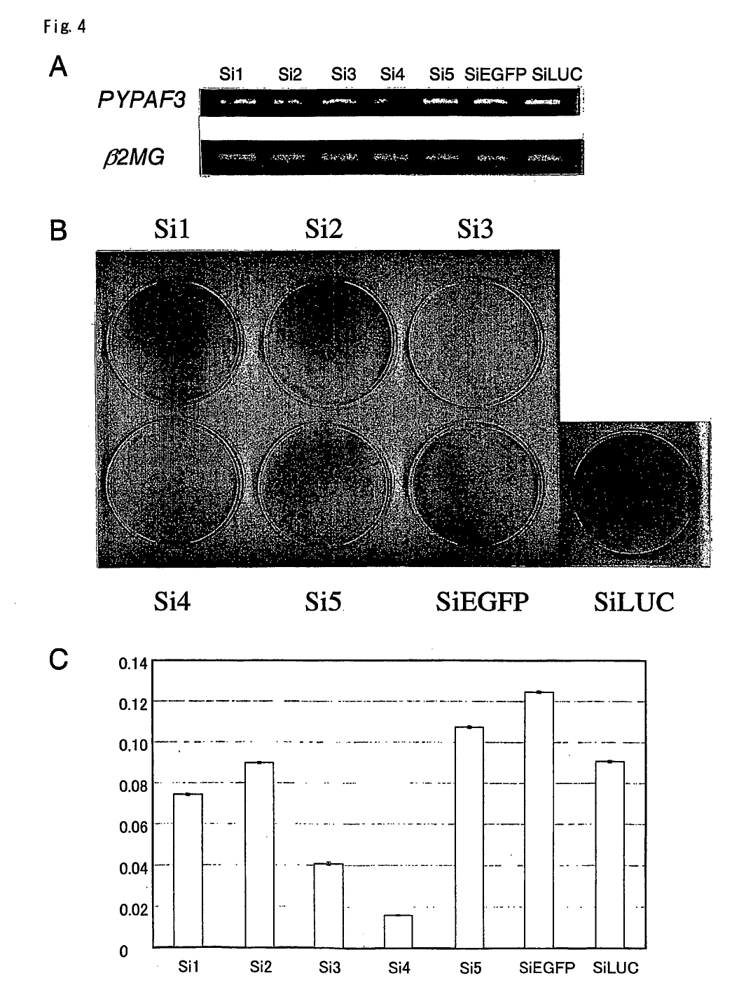

To assess the growth-promoting role of PYPAF3, we knocked down the expression of endogenous PYPAF3 in testicular germ cell tumor line Tera-2 cells, by means of the mammalian vector-based RNA interference (RNAi) technique and examined the effect on cell growth (see Materials and Methods). As shown in FIG. 4a, introduction of psiU6BX-PYPAF3 (Si 4) clearly reduced expression of PYPAF3 transcript in Tera-2 cell lines while no effect was observed in cells transfected with control plasmids (psiU6BX-EGFP and psiU6BX-Luciferase siRNA expression vectors). To confirm the gene-specific growth reduction by psiU6BX-PYPAF3, we performed colony-formation assays of the same two cell lines; as shown in FIG. 4b and 4c, introduction of psiU6BX-PYPAF3 (Si 4) significantly suppressed growth of Tera-2 cells, consisting with the result of above reduced expression, whereas introduction of Si 3 markedly suppressed growth of Tera-2 cells, although knock down of PYPAF3 transcript level showed no almost of reduction. Moreover, MTT assays also indicated significantly growth inhibition of Tera-2 cells when PYPAF3 expression was repressed using psiU6BX-PYPAF3 (Si 3 and Si 4) (FIGS. 4a, b). Each result was verified by three independent experiments.

TABLE 6

Oligonucleotides sequences for small
interference RNA of PYPAF3

SEQ
ID
NO

| | | | |
|---|---|---|---|
| Si1 | Sense | 5′-CACCGAGGCTGATGGCAAGAAACT | 71 |
| | | TCAAGAGAGTTTCTTGCCATCAGCCTC-3′ | |
| | Antisense | 5′-AAAAGAGGCTGATGGCAAGAAACT | 72 |
| | | CTCTTGAAGTTTCTTGCCATCAGCCTC-3′ | |
| Si2 | Sense | 5′-CACCGAGATGAATCTCACGGAATTT | 73 |
| | | CAAGAGAATTCCGTGAGATTCATCTC-3′ | |
| | Antisense | 5′-AAAAGAGATGAATCTCACGGAATTC | 74 |
| | | TCTTGAAATTCCGTGAGATTCATCTC-3′ | |
| Si3 | Sense | 5′-CACCGTAGGACACTTCTTATTCGTT | 75 |
| | | CAAGAGACGAATAAGAAGTGTCCTAC-3′ | |
| | Antisense | 5′-CTCTTGAACGAATAAGAAGTGTCCTAC | 76 |
| | | CTCTTGAACGAATAAGAAGTGTCCTAC-3′ | |
| Si4 | Sense | 5′-CACCGTGATGCATTGTTCCTTCATT | 77 |
| | | CAAGAGATGAAGGAACAATGCATCAC-3′ | |
| | Antisense | 5′-AAAAGTGATGCATTGTTCCTTCATC | 78 |
| | | TCTTGAATGAAGGAACAATGCATCAC-3′ | |
| Si5 | Sense | 5′-CAAGAGAGAGATATCTACAGCCAAGC | 79 |
| | | CAAGAGAGAGATATCTACAGCCAAGC-3′ | |
| | Antisense | 5′-AAAAGCTTGGCTGTAGATATCTCTC | 80 |
| | | TCTTGAAGAGATATCTACAGCCAAGC-3′ | |
| Si- | Sense | 5′-CACCGAAGCAGCACGACTTCTTCT | 81 |
| EGEP | | TCAAGAGAGAAGAAGTCGTGCTGCTTC-3′ | |
| | Antisense | 5′-AAAAGAAGCAGCACGACTTCTTCTCT | 82 |
| | | CTTGAAGAAGAAGTCGTGCTGCTTC-3′ | |
| Si- | Sense | 5′-CACCGTGCGCTGCTGGTGCCAACT | 83 |
| Luci- | | CTCTTGAAGTTGGCACCAGCAGCGCAC-3′ | |
| ferace | Antisense | 5′-AAAAGTGCGCTGCTGGTGCCAACTT | 84 |
| | | CAAGAGAGTTGGCACCAGCAGCGCAC-3′ | |

### INDUSTRIAL APPLICABILITY

The gene-expression analysis of TS described herein, obtained through a combination of laser-capture dissection and genome-wide cDNA microarray, has identified specific genes as targets for cancer prevention and therapy. Based on the expression of a subset of these differentially expressed genes, the present invention provides a molecular diagnostic markers for identifying or detecting TS.

The methods described herein are also useful in the identification of additional molecular targets for prevention, diagnosis and treatment of TS. The data reported herein add to a comprehensive understanding of TS, facilitate development of novel diagnostic strategies, and provide clues for identification of molecular targets for therapeutic drugs and preventative agents. Such information contributes to a more profound understanding of testicular tumorigenesis, and provide indicators for developing novel strategies for diagnosis, treatment, and ultimately prevention of TS.

All patents, patent applications, and publications cited herein are incorporated by reference in their entirety. Furthermore, while the invention has been described in detail and with reference to specific embodiments thereof, it will be apparent to one skilled in the art that various changes and modifications can be made therein without departing from the spirit and scope of the invention.

### REFERENCES

1. Chaganti, R. S. K. and Houldsworth, J. Genetics and Biology of Adult Human male germ Cell Tumors. Cancer Res., 60: 1475-1482, 2000.
2. Bergstorm, R., Adami, H. O., Mohner, M., Zatonski, W., Storm, H., Ekbom, A., Tretli, S., Teppo, L., Akre, O., and Hakulinen, T. Increase in testicular cancer in six European countries: a birth cohort phenomenon. J Natl. Cancer Inst., 88: 727-733, 1996.
3. Zheng, T., Holford, T. R., Ma, Z., Ward, B. A., Flannery, J., and Boyle, P. Continuing increase in incidence in germ cell testis cancer in young adults: experience from Connecticut, USA, 1935-1992. Int. J. Cancer, 65: 723-729, 1996.
4. Dieckmann K P and Pichlmeier U. The prevalence of familial testicular cancer: an analysis of two patient populations and a review of the literature. Cancer 80: 1954-1960, 1997.
5. United Kingdom Testicular Cancer Study Group. Aetiology of testicular cancer: association with congenital abnormalities, age at puberty, infertility, and exercise. Br Med J 308: 1393-1399, 1994.
6. Dong C, Lonnstedt I, and Hemminki K. Familial testicular cancer and second primary cancers in testicular cancer patients by histological type. Eur J Cancer 37: 1878-1885, 2001.
7. Smiraglia, D. J., Szymanska, J., Kraggerud S. M. K., Lothe, R. A., Peltomaki, P., and Plass, C. Distinct epigenetic phenotypes in seminomatous and nonseminomatous testicular germ cell tumors. Oncogene, 21: 3909-3916, 2002.
8. Richie, J. P. Neoplasms of the testis. In: Walsh, P. C., Retik, A. B., Vaughan E. D. Jr., and Wein, A. J. Cambell's Urology Seventh Edition, pp2411-2452. Philadelphia: W.B Sauders Co., 1998
9. Van Brussel, J. P. and Mikisch, G. H. J. Prognostic factors in prostate and testis cancer. BJU International, 83: 910-917, 1999
10. Ottesen A M, Kirchhoff M, De-Meyts E R, Maahr J, Gerdes T, Rose H, Lundsteen C, Petersen P M, Philip J, and Skakkebaek N E. Detection of chromosomal aberrations in seminomatous germ cell tumours using comparative genomic hybridization. Genes Chromosomes Cancer 20: 412-418, 1997.
11. Takayama, H., Takakuwa, T., Tsujimoto, Y., Tani, Y., Nonomura, N., Okuyama, A., Nagata, S., and Aozasa K. Frequent Fas gene mutations in testicular germ cell tumors. Am J Pathol., 161: 635-641, 2002
12. Strohmeyer, T., Reese, D., Press, M., Ackermann, R., Hartmann, M., and Slamon, D.

Expression of the c-kit proto-oncogene and its ligand stem cell factor (SCF) in normal and malignant human testicular tissue. J Urol., 153: 511-515, 1995
13. Skotheim, R. I., Monni, O., Mousses, S., Fossa, S. D., Kallioniemi, O. P., Lothe, R. A., and Kallioniemi, A. New insights into testicular germ cell tumorigenesis from gene expression profiling. Cancer Res., 62: 2359-2364, 2002
14. Shuin T, Misaki H, Kubota Y, Yao M, Hosaka M. Differential expression of protooncogenes in human germ cell tumors of the testis. Cancer 73: 1721-1727, 1994.
15. Van Brussel J P and Mikisch G H J. Prognostic factors in prostate and testis cancer. BJU International 83: 910-917, 1999.
16. Alizadeh, A. A., Eisen, M. B., Davis, R. E., Ma, C., Lossos, I. S., Rosenwald, A., Boldrick, J. C., Sabet, H., Tran, T., Yu, X., Powell, J. I., Yang, L., Marti, G. E., Moore. T., Hudson. J. Jr., Lu, L., Lewis, D. B., Tibshirani. R., Sherlock. G., Chan, W. C., Greiner, T. C., Weisenburger, D. D., Armitage, J. O., Wamnke, R., and Staudt, L. M. Distinct types of diffuse large B-cell lymphoma identified by gene expression profiling. Nature, 403: 503-511, 2000
17. Kihara, C., Tsunoda, T., Tanaka, T., Yamana, H., Furukawa, Y., Ono, K., Kitahara, O., Zembutsu, H., Yanagawa, R., Hirata, K., Takagi, T., and Nakamura, Y. Prediction of sensitivity of esophageal tumors to adjuvant chemotherapy by cDNA microarray analysis of gene-expression profiles. Cancer Res., 61: 6474-6479, 2001
18. Kaneta, Y., Kagami, Y., Katagiri, T., Tsunoda, T., Jin-nai, I., Taguchi, H., Hirai, H., Ohnishi, K., Ueda, T., Emi, N., Tomida, A., Tsuruo, T., Nakamura, Y., and Ohno, R. Prediction of sensitivity to ST1571 among chronic myeloid leulemia patients by genome-wide cDNA microarray analysis. Jpn. J. Cancer Res., 93: 849-856, 2002
19. Yagyu, R., Hamamoto, R., Furukawa, Y., Okabe, H., Yamamuram T., and Nakamura, Y. Isolation and characterization of a novel human gene, VANGL1, as a therapeutic target for hepatocellular carcinoma. Int J Oncol., 20: 1173-1178, 2002
20. Ishiguro H, Shimokawa T, Tsunoda T, Tanaka T, Fujii Y, Nakamura Y, Furukawa Y. Isolation of HELAD1, a novel human helicase gene up-regulated in colorectal carcinomas. Oncogene, 21: 6387-6394, 2002
21. Kitahara, O., Furukawa, Y., Tanaka, T., Kihara, C., Ono, K., Yanagawa, R., Nita, M. E., Takagi, T., Nakamura, Y., and Tsunoda, T. Alterations of gene expression during colorectal carcinogenesis revealed by cDNA microarrays after laser-capture microdissection of tumor tissues and normal epithelia. Cancer Res., 61: 3544-3549, 2001
22. Ono K, Tanaka, T., Tsunoda, T., Kitahara, O., Kihara, C., Okamoto, A., Ochiai, K-, Takagi, T., and Nakamura, Y. Identification by cDNA microarray of genes involved in ovarian carcinogenesis. Cancer Res., 60: 5007-11, 2000
23. Saito-Hisaminato, A., Katagiri, T., Kakiuchi, S., Nakamuram T., Tsunoda, T., and Nakamura, Y. Genome-wide profiling of gene expression in 29 normal human tissues with a cDNA microarray. DNA Res., 9: 35-45, 2002
24. Chuaqui R F, Englert C R, Strup S E, Vocke C D, Zhuang Z, Duray P H, Bostwick D G, Linehan W M, Liotta L A, and Emmert-Buck M R. Identification of a novel transcript up-regulated in a clinically aggressive prostate carcinoma. Urology 50: 302-307, 1997.
25. Baytel, D.; Shalom, S.; Madgar, I.; Weissenberg, R.; and Don, J. The human Pim-2 proto-oncogene and its testicular expression. Biochim. Biophys. Acta, 1442: 274-285, 1998.
26. Kolligs, F. T., Kolligs, B., Hajra, K. M., lIu, G., Tani, M., Cho, K. R., and Fearon, E. R. Ganmma-catenin is regulated by the APC tumor suppressor and its oncogenic activity is distinct from that of beta-catenin. Genes Dev 14: 1319-1331, 2000.
27. Stuart, R. O., Pavlova, A., Beier, D., Li, Z., Krijanovski, Y., and Nigam, S. K. EEG1, a putative transporter expressed during epithelial organogenesis: comparison with embryonic transporter expression during nephrogenesis. Am. J. Physiol. Renal Physiol 281: 1148-1156, 2001.
28. Dhanasekaran S M, Barrette T R, Ghosh D, Shah R, Varambally S, Kurachi K, Pienta K J, Rubin M A, and Chinnaiyan A M, Delineation of prognostic biomarkers in prostate cancer. Nature 412: 822-826, 2001.
29. Jhiang S M. The RET proto-oncogene in human cancers. Oncogene 19: 5590-5597, 2000.
30. Bustelo X R. Regulatory and signaling properties of the Vav family. Mol Cell Biol 20: 1461-1477, 2000.
31. Davies R, Moore A, Schedl A, Bratt E, Miyahara K, Ladomery M, Miles C, Menke A, van Heyningen V, and Hastie N. Multiple roles for the Wilms′ tumor suppressor, WT1. Cancer Res 59 (7 supp) 1747s-1750s, 1999.
32. Kraggerud S M, Skotheim R I, Szymanska J, Eknaes M, Fossa S D, Stenwig A E, Peltomaki P, and Lothe R A. Genome profiles of familial/bilateral and sporadic testicular germ cell tumors. Genes Chromosomes Cancer 34: 168-174, 2002.
33. Morina M A, Codony-Servat J, Albanell J, Rojo F, Arribas J and Baselga J Trastuzumab (herceptin), a humanized anti-Her2 receptor monoclonal antibody, inhibits basal and activated Her2 ectodomain cleavage in breast cancer cells. Cancer Res 61: 4744-4749, 2001.
34. O'Dwyer M E and Druker B J. Status of bcr-abl tyrosine kinase inhibitors in chronic myelogenous leukemia. Curr Opin Oncol 12: 594-597, 2000.
35. Raben D, Helfrich B A, Chan D, Johnson G, and Bunn P A Jr. ZD1839, a selective epidermal growth factor receptor tyrosine kinase inhibitor, alone and in combination with radiation and chemotherapy as a new therapeutic strategy in non-small cell lung cancer. Semin. Oncol 20: 37-46, 2002.
36. Reiser M. and Diehl V. Current treatment of follicular non-Hodgkin's lymphoma. Eur J Cancer 38: 1167-1172,2002.
37. Tschopp J, Martinon F and Burns K. NALPs: a novel protein family involved in inflammation. Nat Rev Mol Cell Biol 4: 95-104, 2002
38. Manji G A, Wang L, Geddes B J, Brown M, Merriam S, Al-Garawi A, Mak S, Lora J M, Briskin M, Jurman M, Cao J, DiStefano PS and Bertin J. PYPAFI, a PYRIN-containing Apaf1-like protein that assembles with ASC and regulates activation of NF-kappa B. J Biol Chem 277: 11570-11575, 2002
39. M. Ishiyama, Y. Miyazono, K. Sasamoto, Y. Ohkura and K. Ueno, A Highly Water-Soluble Disulfonated Tetrazolium Salt as Achromogenic Indicator for NADH as Well as Cell Viability. Talanta 44: 1299-1305, 1997

1. A method of diagnosing TS or a predisposition to developing TS in a subject, comprising determining a level of expression of a TS-associated gene in a patient derived biological sample, wherein an increase or decrease of said level compared to a normal control level of said gene indicates that said subject suffers from or is at risk of developing TS.

2. The method of claim 1, wherein said TS-associated gene is selected from the group consisting of TS 1-346, wherein an increase in said level compared to a normal control level indicates said subject suffers from or is at risk of developing TS.

3. The method of claim 2, wherein said increase is at least 10% greater than said normal control level.

4. The method of claim 1, wherein said TS-associated gene is selected from the group consisting of TS 347-939, wherein a decrease in said level compared to a normal control level indicates said subject suffers from or is at risk of developing TS.

5. The method of claim 4, wherein said decrease is at least 10% lower than said normal control level.

6. The method of claim 1, wherein said method further comprises determining said level of expression of a plurality of TS-associated genes.

7. The method of claim 1, wherein the expression level is determined by any one method select from group consisting of:
(a) detecting the mRNA of the TS-associated genes,
(b) detecting the protein encoded by the TS-associated genes, and
(c) detecting the biological activity of the protein encoded by the TS-associated genes.

8. The method of claim 1, wherein said level of expression is determined by detecting hybridization of a TS-associated gene probe to a gene transcript of said patient-derived biological sample.

9. The method of claim 8, wherein said hybridization step is carried out on a DNA array.

10. The method of claim 1, wherein said biological sample comprises an epithelial cell.

11. The method of claim 1, wherein said biological sample comprises TS cell.

12. The method of claim 8, wherein said biological sample comprises an epithelial cell from a TS.

13. A TS reference expression profile, comprising a pattern of gene expression of two or more genes selected from the group consisting of TS 1-939.

14. A TS reference expression profile, comprising a pattern of gene expression of two or more genes selected from the group consisting of TS 1-346.

15. A TS reference expression profile, comprising a pattern of gene expression of two or more genes selected. from the group consisting of TS 347-939.

16. A method of screening for a compound for treating or preventing TS, said method comprising the steps of:
a) contacting a test compound with a polypeptide encoded by TS 1-939;
b) detecting the binding activity between the polypeptide and the test compound; and
c) selecting a compound that binds to the polypeptide.

17. A method of screening for a compound for treating or preventing TS, said method comprising the steps of:
a) contacting a candidate compound with a cell expressing one or more marker genes, wherein the one or more marker genes is selected from the group consisting of TS 1-939; and
b) selecting a compound that reduces the expression level of one or more marker genes selected from the group consisting of TS 1-346, or elevates the expression level of one or more marker genes selected from the group consisting of TS 347-939.

18. A method of screening for a compound for treating or preventing TS, said method comprising the steps of:
a) contacting a test compound with a polypeptide encoded by selected from the group consisting of TS 1-939;
b) detecting the biological activity of the polypeptide of step (a); and
c) selecting a compound that suppresses the biological activity of the polypeptide encoded by TS 1-346 in comparison with the biological activity detected in the absence of the test compound, or enhances the biological activity of the polypeptide encoded by TS 347-939 in comparison with the biological activity detected in the absence of the test compound.

19. The method of claim 17, wherein said test cell comprises a testicular seminoma cell.

20. A method of screening for compound for treating or preventing TS, said method comprising the steps of:
a) contacting a candidate compound with a cell into which a vector comprising the transcriptional regulatory region of one or more marker genes and a reporter gene that is expressed under the control of the transcriptional regulatory region has been introduced, wherein the one or more marker genes are selected from the group consisting of TS 1-939
b) measuring the activity of said reporter gene; and
c) selecting a compound that reduces the expression level of said reporter gene when said marker gene is an up-regulated marker gene selected from the group consisting of TS 1-346 or that enhances the expression level of said reporter gene when said marker gene is a down-regulated marker gene selected from the group consisting of TS 347-939, as compared to a control.

21. A kit comprising a detection reagent which binds to two or more nucleic acid sequences selected from the group consisting of TS 1-939.

22. An array comprising a nucleic acid which binds to two or more nucleic acid sequences selected from the group consisting of TS 1-939.

23. A method of treating or preventing TS in a subject comprising administering to said subject an antisense composition, said composition comprising a nucleotide sequence complementary to a coding sequence selected from the group consisting of TS 1-346.

24. A method of treating or preventing TS in a subject comprising administering to said subject a siRNA composition, wherein said composition reduces the expression of a nucleic acid sequence selected from the group consisting of TS 1-346.

25. The method of claim 24, wherein said siRNA comprises the nucleotide sequence of SEQ ID NO: 85 or 86 as the target sequence.

26. A method for treating or preventing TS in a subject comprising the step of administering to said subject a pharmaceutically effective amount of an antibody or fragment thereof that binds to a protein encoded by any one gene selected from the group consisting of TS 1-346.

27. A method of treating or preventing TS in a subject comprising administering to said subject a vaccine comprising a polypeptide encoded by a nucleic acid selected from the group consisting of TS 1-346 or an immunologically active fragment of said polypeptide, or a polynucleotide encoding the polypeptide.

28. A method of treating or preventing TS in a subject comprising administering to said subject a compoud that increases the expression or activity of TS 347-939.

29. A method for treating or preventing TS in a subject, said method comprising the step of administering a compound that is obtained by the method according to any one of claims 16-20.

30. A method of treating or preventing TS in a subject comprising administering to said subject a pharmaceutically effective amount of polynucleotide select from group consisting of TS 347-939, or polypeptide encoded by thereof.

31. A composition for treating or preventing TS, said composition comprising a pharmaceutically effective amount of an antisense polynucleotide or small interfering RNA against a polynucleotide select from group consisting of TS 1-346.

32. The composition of claim 31, wherein said small interfering RNA comprises the nucleotide sequence of SEQ ID NO: 85 or 86 as the target sequence.

33. A composition for treating or preventing TS, said composition comprising a pharmaceutically effective amount of an antibody or fragment thereof that binds to a protein encoded by any one gene selected from the group consisting of TS 1-346.

34. A composition for treating or preventing TS, said composition comprising a pharmaceutically effective amount of the compound selected by the method of any one of claims 16-20 as an active ingredient, and a pharmaceutically acceptable carrier.

35. A small interfering RNA, wherein the sense strand thereof comprises the nucleotide sequence of SEQ ID NO: 85 or 86.
